(19) United States
(12) Patent Application Publication
(10) Pub. No.: US 2002/0076693 A1
(43) Pub. Date: Jun. 20, 2002

(54) Novel cell surface receptor for HIV retroviruses, therapeutic and diagnostic uses

(75) Inventors: Ara Hovanessian, Bourg La Reine (FR); Christian Callebaut, San Francisco, CA (US); Bernard Krust, Paris (FR); Etienne Jacotot, Paris (FR); Sylviane Muller, Strasbourg (FR); Jean-Paul Briand, Strasbourg (FR); Gilles Guichard, Wolfisheim (FR)

Correspondence Address: FINNEGAN, HENDERSON, FARABOW, GARRETT &, DUNNER LLP, 1300 I STREET, NW, WASHINGTON, DC, 20005, US

(73) Assignee: Institut Pasteur and Centre National de la Recherche Scientifique

(21) Appl. No.: 09/825,886

(22) Filed: Apr. 5, 2001

Related U.S. Application Data
(62) Division of application No. 09/393,302, filed on Sep. 10, 1999.
Application No. 09/393,302 is a continuation of application No. PCT/EP98/01409, filed on Mar. 12, 1998.
(60) Provisional application No. 60/040,969, filed on Mar. 12, 1997.

(51) Int. Cl. C07K 14/705; A61K 38/17; C12Q 1/70

(52) U.S. Cl. 435/5; 530/350; 514/12

(57) ABSTRACT

The present invention pertains to a novel protein complex receptor for HIV retroviruses, namely the “V3 loop HIV receptor”, which consists in the association of three proteins named P95/nucleolin, P40/PHAPII and P30/PHAPI. The invention also concerns peptidic or non peptidic molecules having the ability to alter and/or prevent the binding of the said novel HIV receptor to the HIV retrovirus, specifically to the gp120 glycoprotein of said HIV retrovirus. The invention is also directed to pharmaceutical and diagnostic compositions containing an effective amount of the molecules altering and/or preventing the binding of the HIV retrovirus to the novel HIV receptor as well as to therapeutic or diagnostic methods using such pharmaceutical or diagnostic composition. The invention also deals with methods of screening new active molecules having the ability to alter and/or prevent the binding of the said novel HIV receptor to the HIV retrovirus, specifically to the gp120 glycoprotein of said HIV retrovirus. Finally, the invention is directed to methods of screening genetic defects in the expression of P95/nucleolin, P40/PHAPII or P30/PHAPI in HIV resistant individuals as well as to specific diagnostic means useful to detect such genetic defects.

### FIELD OF THE INVENTION

The present invention pertains to a new receptor for HIV retroviruses, namely the <<V3 loop HIV receptor >>, which comprises at least one of the three polypeptides related to three proteins named P95[nucleolin], P40[PHAPII] and P30[PHAPI]. The invention also concerns peptidic or non peptidic molecules having the capability to alter and/or prevent the binding of the said novel HIV receptor to the HIV retroviruses, specifically to the envelope glycoprotein of the HIV-2 retrovirus. The invention is also directed to pharmaceutical and diagnostic compositions containing an effective amount of the molecules altering and/or preventing the binding of the HIV retrovirus to the novel HIV receptor as well as to therapeutic or diagnostic methods using such pharmaceutical or diagnostic composition. The invention also deals with methods of screening new active molecules having the ability to alter and/or prevent the binding of the said novel HIV receptor to the HIV retroviruses, specifically to the envelope glycoprotein of said HIV retroviruses. Finally, the invention is directed to methods of screening genetic defects in the expression of P95[nucleolin], P40[PHAPII] or P30[PHAPI] in individuals which survive for a long term to HIV infection or HIV resistant individuals, as well as to specific diagnostic means useful to detect such genetic defects.

### BACKGROUND OF THE INVENTION

HIV is an enveloped virus that infects target cells by the fusion of viral and cellular membranes. This fusion requires first the binding of HIV external and transmembrane envelope glycoprotein complex to the CD4 receptor, and is dependent on the presence of cofactors on the cell surface (for a review see Moore et al., 1993; D'Souza and Harden, 1996). The external envelope glycoprotein contains the binding site for the CD4 receptor and an hypervariable region of about 36 amino acids referred to as the V3 loop (Moore and Nara, 1991). The transmembrane glycoprotein contains a potential fusion peptide at its amino terminus which is implicated in the membrane fusion process (Freed et al., 1991). The external and transmembrane glycoproteins (gp120-gp41 for HIV-1) are associated in a noncovalent manner to generate a functional complex in which the V3 loop plays a critical role (Moore et al., 1993; Moore and Nara,1991). Consequently, it has been proposed that the V3 loop might be implicated in post-CD4 binding events by interacting with some hypothetical cell surface proteins.

Throughout the years, several potential coreceptors of CD4 have been proposed in order to explain why CD4 molecule is essential but not sufficient for HIV entry and infection. By biochemical approaches, various cell surface proteins have been reported to interact with the V3 loop of gp120 and gp41 (Hattori et al., 1989; Kido et al., 1990; Niwa et al., 1996; Avril et al., 1994; Yu et al., 1995; Chin et al., 1995; Chen et al., 1992; Ebenbichler, et al., 1993; Henderson and Quershi, 1993; Quereshi et al., 1990), however, in most cases the relationship between the interaction and a putative role in HIV infection had not been determined. Independent of the V3, the work of other groups has suggested the participation of a cell surface protease similar to trypsin referred to as tryptase TL2 (Kido et al., 1990; Koito et al., 1989), the Fc receptor (McKeating et al., 1990), adhesion molecules LFA-1 (Hildreth and Orentas, 1989; Pantaleo et al., 1991a; 1991b) and ICAM-3 (Sommerfelt and Asjö, 1995), major histocompatibility complex class I and class II molecules (Mann et al., 1988; Corbeau et al., 1991), cell surface antigens CD7 (Sato et al., 1994) and CD44S (Dukes et al., 1995), and finally cell surface membrane associated components such as heparan sulfates (Patel et al., 1993), lectins (Curtis et al., 1992) and glycolipids (Dragic et al., 1995). In CD4 negative cells, galactosyl ceramide has been shown to be the responsible factor for binding of HIV particles (Harouse et al., 1991; Bhat et al., 1991; Fantini et al., 1993). In view of all these observations, it is conceivable that several cell surface antigens may coordinate the complex machinery of membrane fusion process in which the HIV gp120-gp41 envelope complex plays a key role. Furthermore, it is difficult to eliminate the possibility that the requirement for some of the individual components might depend on the cell line studied (Moore et al., 1993; Pantaleo et al., 1991; Callebaut et al., 1994; Callebaut and Hovanessian, 1996). More recently, convincing evidence was provided by several laboratories to show that the G protein-coupled chemokine receptors belonging to the large family of seven-transmembrane-spanning (7tm) cell surface proteins, such as Fusin/CXCR4 and CCR5, serve as species specific, essential cofactors for the entry of T cells and macrophage-tropic HIV-1 isolates, respectively (Feng et al., 1996; Deng et al., 1996; Dragic et al., 1996; Choe et al., 1996; Doranz et al., 1996). Moreover, the cofactor role of these chemokine receptors was shown to be influenced by the presence and the structure of the V3 loop (Choe et al., 1996; Cocchi et al., 1996; Lapham et al., 1996, Wu et al., 1996; Trkola et al., 1996; Oravecz et al., 1996). However, no direct evidence has been provided to suggest that this latter is due to a direct interaction with the V3 loop. Several studies have indicated that the interaction between gp120 and chemokine receptors is a post-CD4 binding event, requiring first the binding of gp120 to CD4 (Lapham et al., 1996, Wu et al., 1996; Trkola et al., 1996).

Previously, the inventors have reported that the “template assembled synthetic peptide” constructs (referred to as TASP), presenting pentavalently the tripeptide KPR or RPK, are potent and specific inhibitors of lymphocyte-tropic HIV entry and infecion (Callebaut et al., 1996). These constructs were designed in order to mimick the conserved RP dipeptide motif, along with basic residues (lysine and arginine) in the V3 loop of HIV isolates (Callebaut et al., 1996; Myers et al., 1994) and were shown to inhibit HIV infection. Nevertheless, the specific cell target of the TASP peptide constructs remained unknown, as well as their exact mode of action when interfering with the HIV entry into the cells.

### SUMMARY OF THE INVENTION

The inventors have now discovered that the FITC-labeled 5[Kψ (CH₂N)PR]-TASP binds specifically to different types of human cells, such as CD4⁺ T cell lines CEM and MOLT4, and PHA-stimulated PBMC, as well as the CD4⁻ Daudi Burkitt's lymphoma cells. Furthermore, the inventors have demonstrated, by ligand blotting experiments, that biotin-labeled 5[Kψ (CH₂N)PR]-TASP specifically binds to a 95-97 kDa molecular weight protein, which is referred to as P95, and form a stable complex with P95. The inventors have also shown that the V3 loop peptide of the HIV1 Lai gp120 is able to bind to the same P95 protein at the cell surface (See FIGS. 1-7 +L).

Now, by using an affinity matrix containing either the 5[Ky (CH₂N)PR]-TASP

pseudopeptide or a synthetic V3 loop peptide, the present inventors have isolated three major proteins referred to as P95, P40 and P30 as components of a novel cellular receptor for HIV (See FIG. 9 +L). Optionally, a P95 derived protein referred to as P60 was also characterized, as detailed herafter.

Microsequencing of peptides from such purified proteins revealed that, P95 is nucleolin, p60 is a partial degradation product of nucleolin, whereas P30 and P40 are recently described proteins named PHAP I and PHAP II, respectively. In a ligand blotting type experiment, both the biotin-labeled 5[Ky(CH₂N)PR]-TASP and the V3 loop peptide were found to bind to P95, P40 and P30. In view of this, it will be referred to P95/nucleolin; P40/PHAP II, and P30/PHAP I as V3-loop binding proteins (V3 loop-BPs). Recombinant envelope glycoprotein of HIV-1, and particularly the gp120 corresponding to several lymphotropic HIV-1 isolates have now been shown to bind with a high affinity the purified preparations of the V3 loop-BPs, containing nucleolin/PHAP II/PHAP I. This binding is inhibited by monoclonal antibodies against the V3 loop. Rabbit polyclonal antibodies raised against synthetic peptides corresponding to the NH₂-terminal sequence of nucleolin, PHAP I and II react specifically with the respective protein, and any one of such antibodies inhibit HIV infection, consistent with the fact that nucleolin/PHAP II/PHAP I are functional in the same complex. The complex of the V3 loop-BPs therefore, represents a receptor of the viral V3 loop and has an essential function in the process of the HIV-induced membrane fusion leading to virus entry and infection.

Therefore, nucleolin/PHAP II/PHAP I are implicated as cofactors in the process of HIV entry in the cells. The cofactor role of nucleolin/PHAP II/PHAP I as V3 loop-BPs in the HIV entry process is enforced by several observations: 1) inhibition of HIV infection using purified preparations of nucleolin/PHAP II/PHAP I; 2) inhibition of HIV entry and infection by antibodies directed against nucleolin/PHAP II/PHAP I; 3) demonstration that gp120 binds nucleolin/PHAP II/PHAP I via its V3 loop; 4) inhibition of gp120 binding by antibodies against nucleolin/PHAP II/PHAP I. These results demonstrate that by virtue to bind the V3 loop domain, nucleolin/PHAP II/PHAP I interact with the gp120 on the surface of HIV particles and thus become implicated in the HIV entry process. Consequently, agents such as the pseudopeptide 5[Kψ(CH₂N)PR]-TASP which bind nucleolin/PHAP II/PHAP I, block the interaction of the V3 loop domain of the envelope glycoprotein of HIV, such as the HIV-1 gp120, with cell surface expressed nucleolin/PHAP II/PHAP I and thus block entry.

Taken together, the inventors results demonstrate that the V3 loop-BPs (nucleolin/PHAP II/PHAP I) constitute new receptors of the V3 loop of gp120 since they serve as cofactors of CD4 in the lymphotropic HIV-1 mediated fusion of virus to cell membranes, leading to HIV entry and infection. The observation that antibodies directed against any one of the V3 loop-BPs are capable in mediating a block of HIV entry and gp120 binding to nucleolin/PHAP II/PHAP I indicate that these three proteins are involved in the same complex. Although each one of the components of this complex can bind the V3 loop as demonstrated by ligand blotting experiments, it cannot be excluded the possibility for the existence of different affinities of binding between the V3 loop and the individual components of the V3 loop-BPs. Consistent with this, the affinity of the pseudopeptide 5[Kψ(CH₂N)PR]-TASP to bind nucleolin is at least two-fold higher compared to that of PHAP II, and the affinity of the same pseudopeptide to bind PHAP II is two-fold higher than PHAP I. Thus it might be possible that the first interaction between the oligomeric gp120 presented by the HIV particles and nucleolin is then followed by the interaction with PHAP II and PHAP I, resulting in the generation of a functional receptor complex for the V3 loop of gp120. In this respect, it is worthwhile to note that the interleukin 2 (IL-2) receptor is composed of three distinct components, the a, the b, and the g chain, of which IL-2 has been shown to bind at different affinities to α and β chains, whereas no specific binding has been shown to occur with the γ chain. The noncovalently associated α, β, and γ chains manifest higher affinity to bind IL-2 compared to that observed for the α and β chains (Waldman, 1993; Taniguchi and Minami, 1993), moreover IL-2 binding has been proposed to induce conformational modifications in the IL-2 receptor complex leading to the generation of a high affinity form which is then functionally competent (Voss et al., 1993).

Consistent with the hypothesis that nucleolin, PHAP II and PHAP I are associated together in a well defined structure, antibodies directed against any one of them inhibited the binding of HIV-Lai particles to CEM cells and thus infection. Interestingly, any one of such antibodies also inhibited infection of peripheral blood mononuclear cells with the macrophage-tropic HIV-1 Ba-L and Ada-M isolates or syncytium- and non-syncytium-inducing primary HIV-1 isolates. Our results suggest that these three V3-BPs serve as an anchorage point besides CD4 for stable binding of HIV particles to permissive cells.

Here, the inventors show that 5[Kψ(CH₂N)PR]-TASP is a potent inhibitor of infection of cells by T lymphocyte and macrophage tropic HIV-1, HIV-2, primary HIV-1, and anti-HIV drug resistant HIV-1 isolates. The binding of 5[Kψ(CH₂N)PR]-TASP to the cell-surface expressed nucleolin results in a specific cleavage of the protein thus confirming that nucleolin should be one of the main targets of this pseudopeptide inhibitor of HIV binding and thus entry.

Consistent with the sequence homologies between the gp120 HIV-1 and the gp125 HIV-2 V3 loop domains, and unambiguously supported by the previous findings of Callebaut et al. (1996, Virology, 218:181-192), who have shown that peptide-TASP constructs were able to inhibit infection of various types of CD4-expressing cells by both HIV-1 Lai and HIV-2 EHO, the V3 loop HIV receptor according to the present invention constitutes a complex receptor for both types of HIV retroviruses.

The discovery by the inventors of a novel cell surface receptor for HIV made between P95/nucleolin, and at least P40/PHAPII and P30/PHAPI proteins or related derivatives constitutes lead them to design new molecules that interact with said receptor or anyone of the protein or polypeptide components consituting said receptor. These molecules are useful as therapeutic agents to prevent or inhibit an HIV infection in vitro and in vivo.

As it will be described in details hreafter, these molecules are either peptidic or non peptidic molecules and are obtained under an isolated or purified form. By <<isolated>> or <<purified>> for the purpose of the present invention is intended that the molecule under consideration has undergone at least one purification or isolation step.

Thus, the present invention concerns a protein complex consisting of at least one of the three following components: P95/nucleolin, P40/PHAPII and P30/PHAPI, or their biologically active derivatives, useful for screening therapeutic molecules active against an HIV infection.

The nucleic sequences and the aminoacid sequences corresponding to P95, P40 and P30 are reported in FIG. 49 +L, respectively in Sections (I-II), III and IV.

From large quantities of purified proteins of human lymphocytic cells, the inventors have performed ligand blotting experiments using either the biotin-labeled 5[Kψ (CH₂N)PR]-TASP or the biotin-labeled V3 loop peptide. By this experimental procedure, it has been shown that each of P95/nucleolin, P40/PHAPII and P30/PHAPI specifically binds to 5[Kψ (CH₂N)PR]-TASP and to the biotin-labeled V3 loop peptide, thus identifying these purified proteins as V3 loop binding proteins (hereafter referred as the V3 loop-BPs). Surprisingly, the V3 loop of gp120 binds to each of the purified protein in the absence of the protein complex formed between the three proteins, thus defining each of P95, P40 and P30 as a ligand of the V3 loop peptide, said ligand having the capability to interact with an envelope glycoprotein, preferably the outer membrane glycoprotein such as gp120 of HIV-1 or gp125 of HIV-2, and prevent the binding of the HIV virus onto the cell surface.

The inventors have shown that 100% saturation of the binding sites are obtained with the following concentrations of the 5[Kψ (CH₂N)PR]-TASP construct, mimicking the V3 loop of the HIV gp120 glycoprotein:

2 μM of 5[Kψ (CH₂N)PR]-TASP for P95/nucleolin;

4 μM of 5[Kψ (CH₂N)PR]-TASP for P40/PHAPII; and

8 μM of 5[Kψ (CH₂N)PR]-TASP for P30/PHAPI.

Furthermore, the inventors have synthesized another type of multibranched peptide (8-Map) containing eight V3 loop consensus motifs (GPGRAF) which was reported to inhibit HIV infection in both CD4+ and CD4− susceptible cells (Yahi et al., 1995). The IC₅₀ of 8-MAP for CEM infection by HIV-1 Lai that are obtained is 25 μM. By FACS nalalysis, the inventors have found that FITC-labeled 8-MAP) binds specifically the surface of different cell lines. Using CEM and C8166 clo,es, it became apparent that the binding pattern of 8-MAP and 5[Kψ (CH₂N)PR]-TASP are very similar. Moreover, in competition experiments, the inventors have showed that 5[Kψ (CH₂N)PR]-TASP is able to block the binding of 8-MAP and vice versa, thus suggesting that 8-MAP and 5[Kψ (CH₂N)PR]-TASP may interact with the same cell surface component, i.e. nucleolin and/or PHAPI and/or PHAPII. It should be noted that 1 μM of 5[Kψ (CH₂N)PR]-TASP or an active derivative is sufficient to block the binding of 10 μM of 8-MAP. This point is probably related to the lower efficacy of 8-MAP to inhibit HIV infection in comparison to 5[Kψ (CH₂N)PR]-TASP. Nevertheless, these results show that peptide constructs derived from other locations of the gp120/gp125 V3 loop domain than 5[Kψ (CH₂N)PR]-TASP may also be involved in the recognition of the cell by the HIV particles.

Consequently, an object of the present invention concerns peptidic or non peptidic molecules that have the ability to inhibit and/or prevent the binding of an HIV retrovirus onto the cells of an individual, specifically an HIV infected patient.

Thus, the present invention covers also compounds that are able to modify the interaction between, on one hand a complex receptor consisting in the association of at least the P95/nucleolin, or P40/PHAPII and/or P30/PHAPI proteins present at the cell surface of a patient infected with a human HIV retrovirus, specifically HIV-1 or HIV-2, and on the other hand the envelope glycoprotein of said HIV retrovirus. The derivatives of the complex receptor are also considered as active molecules that are part of the present invention.

For the purpose of the present invention, the expression (<<envelope glycoprotein>> is not limited in scope to the glycosylated form of the said protein. The expression also embraces the non-glycosylated form of the envelope glycoprotein.

The present invention also concerns structural or functional inhibitor molecules of the HIV envelope glycoprotein, useful to prevent and/or inhibit an infection with a HIV retrovirus.

The invention also concerns the use of the above-defined compounds and inhibitor molecule as active principles of pharmaceutical compositions. The compounds and inhibitor molecules of the present invention are used to prevent the binding of a HIV retrovirus to the cells of an infected patient and/or to inhibit the fusion of cells infected with an HIV retrovirus with (an) unifected cell(s) leading to the formation of syncitia and/or to inhibit the HIV-induced cell death by apoptosis. Such pharmaceutical compositions are useful for treating or preventing an infection with a HIV retrovirus, specifically HIV-1 or HIV-2.

Are also part of the present invention means for screening of molecules that are able to modify the interaction between, on one hand a protein complex receptor consisting in at least P95/nucleolin, or P40/PHAPII or P30/PHAPI proteins or the association of the P95/nucleolin with the P30/PHAPI or the P40/PHAPII or the association of P95/nucleolin with P30/PHAPI and P40/PHAPII and on the other hand the envelope glycoprotein of said HIV retrovirus. The P95/nucleolin, P40/PHAPII or P30/PHAPI proteins are normally present at the cell surface of a patient infected with a human HIV retrovirus, specifically HIV-1 or HIV-2.

### DETAILED DESCRIPTION OF THE INVENTION

The active compounds of the invention have the capability to interact with the part of the HIV envelope glycoprotein without interfering with the natural P95, P40 and P30 located at the cell surface of the cell.

The compounds according to the present invention have the capability to prevent the binding of HIV to the host cells.

By <<inhibitor molecule>> according to the present invention is meant a substance or a group of substances having the ability to alter and/or prevent the recognition of the 5[Kψ (CH₂N)PR]-TASP, the V3 loop peptide, the gp120 HIV glycoprotein or the retrovirus HIV itself by the novel HIV receptor of the invention. By <<inhibitor molecule>> according to the present invention is also intended a substance or a group of substances having the ability to alter and/or prevent the binding of the said receptor of the invention to the 5[Kψ (CH₂N)PR]-TASP, the V3 loop peptide, the gp120 HIV glycoprotein or the retrovirus HIV itself.

Such an inhibitor molecule can block directly the receptor sites, specifically the surface epitopes, that are involved in the interaction with the HIV envelope glycoprotein, either gp120 HIV-1 glycoprotein and gp 125 HIV-2 glycoprotein, for example in that it binds directly to these recognition sites, in place of the V3 loop of the infecting HIV glycoprotein.

Such an inhibitor molecule can also bind to a site of the receptor which is different from the site recognized by the gp120 HIV glycoprotein and induce conformational changes in the receptor molecules such that the receptor is no long able to be recognized by its natural ligand.

The ability of the compounds and inhibitor molecules according to the present invention to alter the interaction between the novel HIV receptor of the invention and the gp120 HIV glycoprotein may be determined by a ligand binding assay or also an ELISA assay, as described in Materials and Methods.

The biological properties of the compounds and inhibitor molecules according to the present invention to alter the interaction between the novel HIV receptor of the invention and the gp120 HIV glycoprotein may also be determined using a method comprising the following steps:

a) bringing into contact cells expressing the novel complex receptor according to the present invention at their surface with an amount of a HIV retrovirus equalling to the TCID₅₀;

b) incubating said cells and retroviruses at 37° C. during a period of time sufficient to allow the entry of the retrovirus within the cells, in the presence of a defined amount of the compound to be assayed;

c) washing the cells in order to remove the retroviruses that has been absorded onto the membranes of the cells;

d) treating the cells in order to eliminate the remaining extracellular retroviruses, for example by a controlled proteolysis with trypsin;

e) preparing cytoplasmic extracts by treating the cells of step d) with an extraction buffer, for example with a buffer containing 20 mM Tris-HCl (pH7.6), 0.15 M NaCl, 5 mM Mg Cl₂, 0.2 mM PMSF, 100 U/ml aprotinin and 0.5% Triton X-100;

f) centrifugating the cells obtained at step c), for example at 1000 g, and harvesting the supernatant medium, in order to separate the retroviral proteins;

g) detecting and optionally measuring the concentration of the HIV proteins, either directly or indirectly, for example by steric hindering.

In a specific embodiment of the above-described method of the invention, step a) is realized using cells bearing at their surface both the novel HIV receptor of the invention and CD4, or fusin or SDF1.

A further object of the present invention consists in the therapeutic application of P95/nucleolin, P40/PHAPII and P30/PHAPI or their biologically active derivatives for preventing an HIV infection, either used each alone or in combination one with another or one with the two others, and optionally also in combination with conventional anti-HIV compounds such as protease inhibitors or nucleotide analogs like AZT or DDI.

The present invention also concerns therapeutic compositions comprising a pharmaceutically effective amount of P95/nucleolin, P40/PHAPII and P30/PHAPI, each protein being used alone or in combination, optionally with one or several pharmaceutically acceptable adjuvants.

Nucleolin is the major non-histone protein of the nucleolus in exponentially growing eukaryotic cells. The deduced amino acid sequence of nucleolin reveals several long stretches of acidic domains rich in aspartate and glutamate residues that has been suggested to be involved in binding to histones. At its C-terminus, there is a glycine-rich domain with the motif GRGG repeated several times which could be implicated in protein-protein and/or protein-nucleic acid interactions (Srivastava et al., 1989). Nucleolin has been implicated in the control of pre-rRNA transcription (Bouche et al., 1984), ribosomal assembly (Bugler et al., 1982), and nucleocytoplasmic transportation of ribosomal components (Borer et al., 1989). In addition to the nucleoli of cells (Pfeifle et al., 1981), nucleolin-like proteins have been shown to be expressed on the cell surface (Pfeifle and Anderer, 1983; Kleinman et al., 1991; Jordan et al., 1994; Krantz et al., 1995). The cell surface expression of nucleolin has been shown to be increased during lymphocyte stimulation and is decreased in differentiated cells (Méhes and Pajor, 1995). More recently, a nucleolin-like protein of 100 kDa Mw has been described to serve as a binding protein for group B coxsackieviruses, but the authors failed to observe binding of Coxsackievirus B to partially purified nuclear nucleolin (Raab de Verdugo et al., 1995). By two dimensional gel isoelectric focusing studies, here the inventors show that nuclear nucleolin is distinct from the protein found in the cytoplasm and on the cell surface. The newly synthesized nucleolin therefore, probably undergoes post-translational modifications which could determine its traficking to the nucleus or to the plasma membrane. Both nuclear and cell surface nucleolin have been reported to be phosphorylated (Belenguer et al., 1990; Jordan et al., 1994), thus other post-translational modifications might account for their distinct resolution in the two dimensional gel isoelectric focusing experiments. In view of these different characteristics of nucleolin, it is tempting to speculate that nucleolin could play other functions in the HIV replication process, besides its function as one of the V3 loop binding proteins.

PHAP I (P30) and PHAP II (P40) had been isolated as putative HLA Class II associated proteins, however as yet there is no direct evidence to elucidate their precise function (Vaesen et al. 1994). The C-termini of PHAP I and PHAP II are composed of a long stretch of acidic amino acids: the last 81 amino acids of PHAP I and the last 54 amino acids of PHAP II, contain 70 and 80% aspartate or glutamate residues, respectively. Vaesen et al. (1994) have proposed that PHAP I and PHAP II might be involved in the generation of intracellular signalling events that lead to regulation of transcriptional events after binding of a ligand to HLA class II molecules. PHAP I is most likely the human homologue of the rat “leucine-rich acidic nuclear protein” (Marsuoka et al., 1994), whereas PHAP II is identical to a protein named SET (Von Lindern et al., 1992).

Although there is no apparent sequence homology between nucleolin/PHAP II/PHAP I, the common feature between these three proteins is their polyanionic nature in virtue of the expression of the extended stretches of acidic amino acids. These domains are probably responsible for the interaction with the V3 loop peptide or the pseudopeptide 5[Ky(CH₂N)PR]-TASP. In this respect, it is worthwhile to mention here that polyanions such as heparin, dextran sulfate, synthetic double-stranded RNAs, synthetic aspartate/glutamate-rich peptides, are potent inhibitors of HIV entry and infection (Krust et al., 1993; Javaherian and McDanal, 1995; Leydet et al., 1996). The mechanism of the inhibitory effect of polyanions has been proposed to be related to their capacity to bind the V3 loop domain in gp120 (Harrap et al., 1994; Javaherian and McDanal, 1995). These observations and the data indicating that gp120 binds the V3 loop-BPs through its V3 loop domain, suggest that polyanions inhibit HIV infection by binding to the basic motifs in gp120 (found in the V3 loop) and thus blocking gp120-interaction with nucleolin/PHAP II/PHAP I.

Considering the ability of each of P95/nucleolin, P40/PHAPII and P30/PHAPI proteins to inhibit the binding of HIV to the cell surface, it is a further object of the present invention to provide with peptide fragments of P95/nucleolin, P40/PHAPII and P30/PHAPI that are able to prevent the binding of HIV to the protein complex receptor of the invention, said peptide fragments being useful as therapeutic agents against an HIV infection.

Peptide fragments of each of the three V3 loop Bps according to the invention may be obtained by the one skill in the art from the aminoacid sequences of P95/nucleolin, P40/PHAPII and P30/PHAPI that are reported in FIG. 49 +L.

Consequently, are also part of the present invention peptide fragments of the P95/nucleolin, P40/PHAPII and P30/PHAPI proteins that may be obtained by cleavage of said proteins with a proteolytic enzyme such that trypsin, chymotrypsine, collagenase, clostripaine, Myxobacter protease, thiol proteases, Proline endopeptidase, Staphylococcal protease, trypsin having the lysine residues blocked, trypsin having the arginine residues blocked or the endoproteinase Asp-N. Fragments of the polypeptides according to the invention may also be obtained by placing the polypeptide in a very acid solution (pH 2.5) or by cleavage using chemical reagents such as cyanogen bromide or iodobenzoate.

For example, P95/nucleolin has 18 potential dibasic cleavage sites (15, 51, 54, 62, 70, 79, 87, 95, 109, 124, 141, 219, 279, 281, 294, 387, 545 and 702; Srivastava et al., 1989), the site at position 545 being unique to human nucleolin. Moreover, nucleolin has been described to be highly susectible to degradation. Polypeptides with masses of 80, 70, 60 and 50 kDa have been identified with antisera to nucleolin and it was suggested that these presumed nucleolin fragments resulted from thiol protease cleavage (Bugler et al., 1982).

P30/PHAPI contains five tyrosine residues (aminoacid positions 131, 148, 163, 179 and 214) which are flanked by acidic residues and thus are potential substrates for tyrosine kinases (Vaesen et al., 1994).

Preferred peptide fragments according to the present invention are the fragments that bind to the 5[Kψ (CH₂N)PR]-TASP or to the V3 loop peptide. Alternatively, said peptide fragments are recognized by antibodies directed respectively to P95/nucleolin, P40/PHAPII or P30/PHAPI proteins, such as the antibodies described in Materials and Methods. Such peptide fragments have advantageously a length of at least 20 aminoacids.

Are also part of the present invention polypeptides that are homologous to any of the P95/nucleolin, P40/PHAPII and P30/PHAPI proteins or the above defined P95/nucleolin, P40/PHAPII and P30/PHAPI peptide fragments. By homologous peptide according to the present invention is meant a polypeptide containing one or several aminoacid additions, deletions and/or substitutions in the aminoacid sequence of either P95/nucleolin, P40/PHAPII and P30/PHAPI proteins. In the case of an aminoacid substitution, one or several-consecutive or non-consecutive-aminoacids are replaced by <<equivalent>> aminoacids. The expression <<equivalent>> aminoacid is used herein to name any aminoacid that may substituted for to one of the aminoacids belonging to the initial polypeptide structure without decreasing the binding properties of the corresponding peptides to the 5[Kψ (CH₂N)PR]-TASP, the V3 loop peptide or the gp120 of HIV-1 or the gp125 of HIV-2. In other words, the <<equivalent>> aminoacids are those which allow the generation or the obtention of a polypeptide with a modified sequence as regards to the aminoacid sequence of P95/nucleolin, P40/PHAPII and P30/PHAPI proteins, the said modified polypeptide being able to bind to the 5[Kψ (CH₂N)PR]-TASP, the V3 loop peptide or the gp120/gp125 proteins of HIV and/or to induce antibodies recognizing the parent polypeptide consisting in any of the P95/nucleolin, P40/PHAPII and P30/PHAPI proteins.

These equivalent aminoacyles may be determined either by their structural homology with the initial aminoacyles to be replaced, by the similarity of their net charge, and optionally by the results of the cross-immunogenicity between the parent peptides and their modified counterparts.

The peptides containing one or several <<equivalent>> aminoacids must retain their specificty and affinity properties to the biological targets of the parent protein, as it can be assessed by a ligand binding assay or an ELISA assay.

By modified aminoacid according to the present invention is also meant the replacement of a residue in the L-form by a residue in the D form or the replacement of a Glutamic acid (E) residue by a Pyro-glutamic acid compound. The synthesis of peptides containing at least one residue in the D-form is, for example, described by Koch et al. in 1977.

As an illustrative example, it should be mentioned the possibility to realize substitutions without a deep change in the V3 loop binding properties of the correspondant modified peptides by replacing, for example, leucine by valine, or isoleucine, aspartic acid by glutamic acid, glutamine by asparagine, arginine by lysine etc., it being understood that the reverse substitutions are permitted in the same conditions.

In order to design peptides homologous to the P95/nucleolin, P40/PHAPII and P30/PHAPI proteins or their peptide fragments, the one skill in the art can also refer to the teachings of Bowie et al. (1990).

A specific, but not limitative, embodiment of a modified peptide molecule of interest according to the present invention, which consists in a peptide molecule which is resistant to proteolysis, is a peptide in which the —CONH— peptide bound is modified and replaced by a (CH₂NH) reduced bound, a (NHCO) retro inverso bound, a (CH₂—O) methylene-oxy bound, a (CH₂—S) thiomethylene bound, a (CH₂CH₂) carba bound, a (CO—CH₂) cetomethylene bound, a (CHOH—CH₂) hydroxyethylene bound), a (N—N) bound, a E-alcene bound or also a —CH═CH— bound.

As it has been already mentioned hereinbefore, the P95/nucleolin, P40/PHAPII and P30/PHAPI proteins share a common feature which consists in their polyanionic regions in virtue of the extended stretches of acidic aminoacids. Such domains are, with a good probability, responsible for the interaction with the V3 loop peptide or with the 5[Kψ (CH₂N)PR]-TASP pseudopeptide. A strong support for this is that polyanions have been shown to be potent inhibitors of HIV entry through their potential capacity to interact with the V3 loop domain (Javaherian at al., 1995; Leydet et al., 1996).

Specifically the P95/nucleolin, P40/PHAPII and P30/PHAPI proteins contain long stretches of acidic aminoacid essentially composed of E (glutamic acid) and D (Aspartic acid) aminoacids.

More specifically, the P95/nucleolin, which has a length of 707 aminoacids, contains at least four sequences almost containing D and E aminoacids, namely:

the sequence beginning at the aminoacid in position 22 and ending at the aminoacid in position 44;

the sequence beginning at the aminoacid in position 143 and ending at the aminoacid in position 171 (89.3% E or D residues);

the sequence beginning at the aminoacid in position 185 and ending at the aminoacid in position 209 (94.5% E or D residues);

the sequence beginning at the aminoacid in position 234 and ending at the aminoacid in position 271 (100% E or D residues);

The P40/PHAPII contains at least one sequence almost containing D and E aminoacids, namely:

the sequence beginning at the aminoacid in position 223 and ending at the aminoacid in position 277 (80% E or D residues);

P30/PHAPI has 249 aminoacids and the C-terminal end of this protein (from position 168 to the end) consists in 80% E or D residues.

The P30/PHAPI contains at least three sequences almost containing D and E aminoacids, namely:

the sequence beginning at the aminoacid in position 168 and ending at the aminoacid in position 182;

the sequence beginning at the aminoacid in position 187 and ending at the aminoacid in position 222;

the sequence beginning at the aminoacid in position 240 and ending at the aminoacid in position 249; it being understood that the proximity of the two first sequences and the two last sequences allow one of ordinary skill in the art to gather the sequences contained in two sets of sequences as follows:

the sequence beginning at the aminoacid in position 168 and ending at the aminoacid in position 222;

the sequence beginning at the aminoacid in position 187 and ending at the aminoacid in position 249;

The above-described E/D rich sequences are thus preferred peptides according to the present invention, useful as inhibitors of the HIV binding to the novel receptor complex composed of the P95/nucleolin, P40/PHAPII and P30/PHAPI proteins.

The peptides used according to the present invention may be prepared in a conventional manner by peptide synthesis in liquid or solid phase by successive couplings of the different aminoacid residues to be incorporated (from the N-terminal end to the C-terminal end in liquid phase, or from the C-terminal end to the N-terminal end in solid phase) wherein the N-terminal ends and the reactive side chains are previously blocked by conventional groups.

For solid phase synthesis the technique described by Merrifield may be used in particular. Alternatively, the technique described by Houbenweyl in 1974 may also be used.

In order to produce a peptide chain using the Merrifield process, a highly porous resin polymer is used, on which the first C-terminal aminoacid of the chain is fixed. This aminoacid is fixed to the resin by means of its carboxyl groups and its amine function is protected, for example, by the t-butyloxycarbonyl group.

When the first C-terminal aminoacid is thus fixed to the resin, the protective group is removed from the amine function by washing the resin with an acid. If the protective group for the amine function is the t-butyloxycarbonyl group, it may be eliminated by treating the resin with trifluoroacetic acid.

The second aminoacid which supplies the second residue of the desired sequence is then coupled to the deprotected amine function of the first C-terminal aminoacid fixed to the chain. Preferably, the carboxyl function of this second aminoacid is activated, for example, using dicyclohexylcarbodiimide, and the amine function is protected, for example, using t-butyloxycarbonyl.

In this way, the first part of the desired peptide chain is obtained, which comprises two aminoacids and the terminal amine function of which is protected. As before, the amine function is deprotected and the third residue can then be fixed, under similar conditions, to those used in the addition of the second C-terminal aminoacid.

Thus, the aminoacids which are to form the peptide chain are fixed, one after another, to the amine group, which is previously deprotected each time, of the portion of the peptide chain already formed, which is attached to the resin.

When all the desired peptide chain is formed, the protecting groups are eliminated from the various amimoacids which constitute the peptide chain and the peptide is detached from the resin, for example using hydrofluoric acid.

The peptides thus synthesized may also be a polymer of the peptide of interest, that contains 2 to 20 monomer units of the aminoacid sequence of interest derived from the aminoacid sequence of either P95/nucleolin, P40/PHAPII and P30/PHAPI, preferably 4 to 15 monomer units and more preferably 5 to 10 monomer units. The said polymers may be obtained by the technique of Merrifield or any other conventional peptide polymer synthesis method well known by the one skill in the art.

The peptides thus obtained may be purified, for example by high performance liquid chromatography, such as reverse phase and/or cationic exchange HPLC, as described by Rougeot et al. in 1994.

The peptides or pseudopeptides according to the present invention is advantageously combined with or contained in an heterologous structure, or polymerized in such a manner as to enhance its ability to prevent HIV binding to the cell, specificaly to the V3 loop receptor of the invention.

As an illustrative embodiment, the peptides or pseudopeptides of the invention are embedded within a peptidic synthetic matrix in order to form a MAP (Multi-branched Associated Peptide) type structure. Such MAP structures as well as their method of preparation are described by Tam in 1988 or in the PCT patent application No. WO94/28915 (Hovanessian et al.). The embedding of the peptides or pseudopeptides of therapeutic value according to the present invention within MAP type structures are expected to cause an increase in the inhibitory properties of the initial molecules as regards to the HIV infection.

Are also part of the present invention peptides or pseudopeptides that contain at least two units (i.e. motifs) of the peptide fragments of the P95/nucleolin, P40/PHAPII or P30/PHAPI protein, or their pseudopeptide counterparts, that have been selected for their specific binding to the 5[Kψ (CH₂N)PR]-TASP construct, the V3 loop peptide or the gp120 HIV glycoprotein, as described above. For the purpose of the present invention, such peptides or pseudopeptides containing more than one unit of a peptide fragment of the P95/nucleolin, P40/PHAPII and P30/PHAPI protein, or their pseudopeptide counterparts, will be termed <<oligomeric peptides or pseudopeptides of the invention>>.

Advantageously, the oligomeric peptides or pseudopeptides defined herein above comprise from 2 to 20 units, preferably from 2 to 12 units and more preferably from 2 to 5 units of the peptide fragments of the P95/nucleolin, P40/PHAPII or P30/PHAPI protein.

In a specific embodiment of the oligomeric peptides or pseudopeptides according to the present invention, they contain repeated unique units consisting in a single selected peptide or pseudopeptide fragment of the P95/nucleolin, P40/PHAPII or P30/PHAPI protein.

In another specific embodiment of the oligomeric peptides or pseudopeptides of the invention, they contain several different units consisting in different selected peptide or pseudopeptide fragments of the P95/nucleolin, P40/PHAPII or P30/PHAPI protein.

Preferably, the units constitutive of the oligomeric peptides or pseudopeptides according to the present invention are choosen among the acidic aminoacid stretches contained in the P95/nucleolin, P40/PHAPII or P30/PHAPI protein that are described in detail hereinbefore.

As an alternative embodiment, the different units contained in the oligomeric peptides or pseudopeptides of the invention are derived from a single protein choosen among the P95/nucleolin, P40/PHAPII and P30/PHAPI proteins or comprise monomer units derived from two or three proteins choosen among P95/nucleolin, P40/PHAPII and P30/PHAPI proteins.

A preferred oligomeric peptide or pseudopeptide according to the present invention comprises a peptide consisting in a sequence choosen among the following sequences:

the sequence of P95/nucleolin beginning at the aminoacid in position 234 and ending at the aminoacid in position 271;

the sequence of P40/PHAPII beginning at the aminoacid in position 223 and ending et the aminoacid in position 277;

the sequence of P30/PHAPI beginning at the aminoacid in position 187 and ending at the aminoacid in position 249.

Alternatively, an oligomeric peptide or pseudopeptide according to the invention comprises the following constructs:

the above described sequence of P95/nucleolin placed in tandem with the above described sequence of P40/PHAPII;

the above described sequence of P95/nucleolin placed in tandem with the above described sequence of P30/PHAPI;

the above described sequence of P30/PHAPI placed in tandem with the above described sequence of P40/PHAPII;

it being understood that said oligomeric peptide or pseudopeptide may contain each particular sequence repeated several times in the molecule, for example from 2 to 10 times and more preferably from 2 to 5 times.

The peptides used in the therapeutic method according to the present invention may also be obtained using genetic engineering methods. The nucleic sequences of the genomic DNA or cDNA encoding the P95/nucleolin protein, and of the cDNA encoding P40/PHAPII and P30/PHAPI proteins are represented in FIG. 49 . For the peptide fragments of interest of the P95/nucleolin, P40/PHAPII and P30/PHAPI proteins, the one skill in the art will refer to the general literature to determine which appropriate codons may be used to synthesize the desired peptide.

There is no need to say that the expression of the polynucleotide that encodes the complete peptide or peptide fragments of interset of P95/nucleolin, P40/PHAPII and P30/PHAPI proteins may be optimized, according to the organism in which the sequence has to be expressed and the specific codon usage of this organism (mammal, plant, bacteria etc.). For bacteria and plant, respectively, the general codon usages may be found in the European Patent Application No EP-0359472 (Mycogen).

It is now easy to produce proteins in high amounts by the genetic engineering techniques by the use, as expression vectors, plasmids, phages or phagemids. The polynucleotides that code for the polypeptides of the present invention is inserted in an appropriate expression vector, in a site non essential for its replication, in order to in vitro produce the polypeptide of interest. Advantageously, the heterologous gene to be expressed is placed under the control of the suitable expression regulation signals for an optimal expression of the heterologous gene in a selected cell host. Consequently, the present invention also embraces the production by genetic engineering techniques of the P95/nucleolin, P40/PHAPII and P30/PHAPI protein, as well as a family of recombined vectors characterized in that they carry at least a polynucleotide coding for the P95/nucleolin, P40/PHAPII or P30/PHAPI protein or one of their peptide fragments.

Thus, a method for producing the P95/nucleolin, P40/PHAPII or P30/PHAPI protein, or one of their peptide fragments binding to the V3 loop of the gp120 HIV glycoprotein (also termed herein <<biologically active derivatives of the P95/nucleolin, P40/PHAPII or P30/PHAPI protein) or also a peptide counterpart of the latters containing <<equivalent>> aminoacids as described above comprises the steps of:

a) Optionally amplifying the nucleic acid coding for the desired polypeptide using a pair of primers specific for the P95/nucleolin, P40/PHAPII and P30/PHAPI genomic or cDNA sequence (by SDA, TAS, 3SR NASBA, TMA, LCR, RCR, CPR, Q-beta replicase or PCR);

b) Inserting the nucleic acid coding for P95/nucleolin, P40/PHAPII and P30/PHAPI protein or one of its peptide fragments of interest in an appropriate vector,

c) culturing, in an appropriate culture medium devoid of serum, a cell host previously transformed or transfected with the recombinant vector of step b);

e) harvesting the culture medium thus conditioned and the cell host, for example by lysing the cell host by sonication or by an osmotic shock;

f) separating or purifying, from the said culture medium, or from the pellet of the resultant host cell lysate the thus produced polypeptide of interest.

g) Characterizing the produced protein or peptide of interest.

h) Optionally assaying for the specific recognition of the said peptide by a polyclonal or a monoclonal antibody directed against the P95/nucleolin, P40/PHAPII and P30/PHAPI protein.

The PCR amplification reaction is described by Saiki et al. in 1985; The SDA technique is described by Walker et al. in 1992 and was improved by Spargo et al. in 1996; The TAS amplification reaction is described by Kwoh et al. in 1989; The 3SR technique is described by Guatelli et al. in 1990; The NASBA technique is described by Kievitis et al. in 1991; The LCR reaction is described by Landergen in 1991 and improved by Barany et al. in 1991; The RCR technique is described by Segev in 1992; The CPR technique is described by Duck et al. in 1990.

The polynucleotides to be expressed as coding for a peptidic therapeutic molecule according to the present invention may be obtained by cleavage of the genomic or the cDNA of P95/nucleolin, P40/PHAPII or P30/PHAPI by restriction endonucleases. The conditions under which the restrictions enzymes are used in order to generate the polynucleotide fragments according to the invention are described in Sambrook et al., 1989.

The suitable promoter regions used in the expression vectors according to the present invention are choosen taking into account of the cell host in which the heterologous gene has to be expressed.

Preferred bacterial promoters are the LacI, LacZ, the T3 or T7 bacteriophage RNA polymerase promoters, the polyhedrin promoter, or the p10 protein promoter from baculovirus (Kit Novagen) (Smith et al., 1983; O'Reilly et al., 1992), the lambda P_{R} promoter or also the trc promoter.

Preferred promoter for the expression of the heterologous gene in eukaryotic hosts are the early promoter of CMV, the Herpes simplex virus thymidine kinase promoter, the early or the late promoter from SV40, the LTR regions of certain retroviruses or also the mouse metallothionein I promoter.

The choice of a determined promoter, among the above-described promoters is well in the ability of one skill in the art, guided by his knowledge in the genetic engineering technical field, and by being also guided by the book of Sambrook et al. in 1989 or also by the procedures described by Fuller et al. in 1996.

Generally, suitable expression vectors used according to the present invention embrace plasmids, phages, cosmids or phagemids.

A suitable vector for the expression of the P95/nucleolin, P40/PHAPII and P30/PHAPI protein above-defined or their peptide fragments is baculovirus vector that can be propagated in insect cells and in insect cell lines. A specific suitable host vector system is the pVL1392/1393 baculovirus transfer vector (Pharmingen) that is used to transfect the SF9 cell line (ATCC NoCRL 1711) which is derived from Spodoptera frugiperda.

Other suitable vectors for the expression of the P95/nucleolin, P40/PHAPII and P30/PHAPI protein above-defined or their peptide fragments in a baculovirus expression system consist in plasmids which are baculovirus expression vectors with multiple cloning sites (MCS) that contain the specific expression elements of the pol gene in a pUC8 backbone. These plasmids can be divided into two subgroups, namely, on one hand the vectors pVLMelMyc-, which allow the construction of a N-terminal function to the signal sequence of the melittin gene (Chai et al., 1993; Vlasak et al., 1983) and on the other hand the vectors pVLPolMyc- which allow a N-terminal fusion to the first 12 aa of the pol and the c-Myc tag. The gene to be expressed can be cloned into the MCS, resulting in an N-terminal fusion to either the mel-myc or the pol-myc which are encoded by the vectors. An example of using such versatile vectors to express a mouse heterologous protein (5HT_{5A} serotonin receptor) is notably described by Lenhardt et al. in 1996.

Another suitable vector for performing the above-described process is a vaccinia virus vactor. In this specific embodiment, BSC-40 or LoVo are used for the transfection and culture steps.

Other particular expression vectors are the followings:

a) bacterial vectors: pBs, phagescript, PsiX174, pBluescript SK, pNH8a, pNH16a, pHN18a, pNH46a (all commercialized by Stratagene); pTrc99A, pKK223-3, pDR540, pRIT5 (all commercialized by Pharmacia); baculovirus transfer vector pVL1392/1393 (Pharmingen); pQE-30 (QIAexpress).

b) eukaryotic vectors: pWLneo, pSV2cat, pOG44, pXT1, pSG (all commercialized by Stratagene); pSVK3, pBPV, pMSG, pSVL (all commercialized by Pharmacia).

All the above-described vectors are useful to transform or transfect cell hosts in order to express the polynucleotide coding for the P95/nucleolin, P40/PHAPII or P30/PHAPI proteins or their peptide fragments or also the different oligomeric peptides according to the present invention.

A cell host according to the present invention is characterized in that its genome or genetic background (including chromosome, plasmids) is modified by the heterologous coding for the P95/nucleolin, P40/PHAPII or P30/PHAPI proteins or their peptide fragments or also the different oligomeric peptides according to the present invention.

Preferred cell hosts used as recipients for the expression vectors of the invention are the followings:

a) Prokaryotic cells: Escherichia coli strains (I.E. DH5-α strain) or Bacillus subtilis.

b) Eukaryotic cell hosts: HeLa cells (ATCC NoCCL2; NoCCL2.1; NoCCL2.2), Cv 1 cells (ATCC NoCCL70), COS cells (ATCC NoCRL1650; NoCRL1651), Sf-9 cells (ATCC NoCRL1711).

The purification of the recombinant protein, peptide or oligomeric peptide according to the present invention may be realized by passage onto a Nickel or Cupper affinity chromatography column. The Nickel chromatography column may contain the Ni-NTA resin (Porath et al., 1975).

The peptides produced by genetic engineering methods according to the invention may be characterized by binding onto an immunoaffinity chromatography column on which polygonal or monoclonal antibodies directed to P95/nucleolin, P40/PHAPII or P30/PHAPI have previously been immobilized.

More preferably, the peptide of therapeutic value contained in the therapeutic compositions according to the present invention are purified by HPLC as described by Rougeot et al. in 1994. The reason to prefer this kind of peptide or protein purification is the lack of side products found in the elution samples which renders the resultant purified protein or peptide more suitable for a therapeutic use.

Another embodiment of the peptide molecules according to the present invention that have the ability to modify the interaction between, on one hand a protein complex receptor consisting in the association of the P95/nucleolin, P40/PHAPII and P30/PHAPI proteins present at the cell surface of a patient infected with a human HIV retrovirus (namely the V3 loop HIV receptor), specifically HIV-1 or HIV-2, and on the other hand the gp120 envelope glycoprotein of said HIV retrovirus, consists in polyclonal or monoclonal antibodies.

A first embodiment of such antibodies consists in that they have the ability to block the binding of 5[Kψ (CH₂N)PR]-TASP construct, of the V3 loop peptide, of the HIV gp120/gp125 glycoproteins or of the HIV virus to said receptor, either by interacting directly with the receptor sites specific for HIV gp120 or by interacting with other sites that will induce conformational changes of the receptor that greatly diminishes or completely abolishes the receptor ability to bind to HIV.

Such antibodies according to this specific embodiment are, for example the monoclonal antibody directed to the P95nucleolin described by Chen et al. (1991) or by Fang et al. (1993) or also the polyclonal antibodies directed to the P95/nucleolin that are described in Section I.A. of Materials and Methods. Monoclonal or polyclonal antibodies directed against the P40/PHAPII or the P30/PHAPI proteins are prepared according to the procedures described by Chen et al. (1991) or by Fang et al. (1993).

A second embodiment of such antibodies consist in that they have the ability to block the binding of 5[Kψ (CH₂N)PR]-TASP construct, of the V3 loop peptide, of the HIV gp120/gp125 glycoproteins or of the HIV virus to said receptor either by interacting directly with the gp120/gp125 sites specifically recognized by the V3 loop HIV receptor or by interacting with other sites of gp120 that will induce conformational changes within said HIV glycoprotein that greatly diminishes or completely abolishes the receptor ability to bind to HIV.

Such antibodies according to this specific embodiment are, for example the monoclonal antibody N11/20 directed against the V3 loop of gp120, Mab 110/C directed against an epitope in gp120 corresponding to fragment 282-284 aminoacids, Mab 110/D directed against an epitope of gp120 situated at residues 381-394, mAb 41-A directed both against gp41 and gp120 and Mab 125-A directed against the external envelope glycoprotein of HIV-2 (All Mab being publicly available from Hybridolab, Institut Pasteur, Paris, France). Other suitable antibodies are Mab 110-4 directed against the gp120 V3 loop and Mab 110-1 directed against the C-terminal domain of gp120 (those Mab being respectively described by Kinney-Thomas et al., 1988; Linsley et al., 1988 and which are commercially available from Genetics Systems, Seattle, Wash.). Are also preferred antibodies according to the present invention Mab ADP390 directed against the CD4 binding domain in gp120 (Mc Keating et al., 1992), Mab AD3 directed against the first 204 aminoacids od gp120, mAb V3-21 against the INCTRPN sequence et residues 298-304 containing the N-terminal end of V3 loop and Mab b12 directed against the CD4 binding domain in gp120, those antibodies being described in Section I.A. of Materials and Methods.

A third embodiment of the antibodies of therapeutic value according to the present invention consists in anti-idiotypic antibodies that mimmick the P95/nucleolin, the P40/PHAPII or the P30/PHAPI proteins. Such anti-idiotypic antibodies may be prepared using, as starting material, monoclonal antibodies directed to a protein choosen among the P95/nucleolin, P40/PHAPII and P30/PHAPI proteins or one of their peptide or pseudopeptide fragments that are described above. Such anti-idiotypic antibodies that mimmick the the P95/nucleolin, the P40/PHAPII or the P30/PHAPI proteins may be prepared according to the procedures described by Perosa et al., 1996, Deckert et al., 1996, Polonelli et al. or 1996, Barchan et al., 1995.

As an ilustrative but not limitative example, purified monoclonal antibodies, for example the monoclonal antibody directed to the P95/nucleolin described by Chen et al. (1991) or by Fang et al. (1993), which will be named Mab P95, are adsorbed to aluminium phosphate and injected to mouse s.c. on days 0, 21 and 42. An additional injection is given on day 80-100.

Then, purified Mab P95 are conjugated to Affi-Gel (Bio-Rad Laboratories, Richmond, Calif.) (5-10 mg/ml gel) following the manufacturer's instructions. Serum samples from Mab P95 immunized mice are repeatedly adsorbed on a unrelated mouse monoclonal antibody-conjugated column (previously equilibrated with PBS) until all detectable anti-isotypic and anti-allotypic antibodies are removed.

The eluted fractions of sera are then passed onto a Mab anti-P95/nucleolin-conjugated column in order to retain the desired anti-idiotypic antibodies. Bound antibodies are then eluted with 0.1 M glycine, pH 2.9 neutralized with 1 M. Tris and dialyzed overnight against PBS.

The resultant anti-idiotypic antibodies are then assayed for specific binding to the V3 loop peptide using a ligand binding experimental procedure or an ELISA assay with an immobilized V3 loop peptide, preferably a competition ELISA assay using also non labeled P95/nucleolin as the competitor compound.

The whole embodiments of the above-described antibodies are also part of the present invention, excepted for the antibodies that were already known in prior art, the latters being only part of the invention as active principles of the anti-HIV therapeutic compositions of the invention.

In vivo, number of patients infected with HIV produce a significant level of antibodies directed to the V3 loop domain of gp120/gp125. Consequently, these patients sera may also contain the anti-idiotypic antibodies counterpart, part of them recognizizng at least one epitope of a protein choosen among P95/nucleolin, P40/PHAPII or P30/PHAPI protein. Thus, it would be very useful to screen HIV-infected patients sera for anti-idiotypic antibodies that are able to bind to at least one of the P95/nucleolin, P40/PHAPII or P30/PHAPI proteins and to determine the specific epitope(s) recognized by such patients sera antibodies, in relation with the development of the disease. By such a screening method, it will allow the practitioner to identify specific epitopes of P95/nucleolin, P40/PHAPII or P30/PHAPI protein which are associated with the presence of neutralizing antibodies in the patients sera and thus allow the definition of specific preferred epitopes of P95/nucleolin, P40/PHAPII or P30/PHAPI protein to include or mimick in the therapeutic molecules according to the present invention.

The screening for these <<natural>> anti-idiotypic antibodies found in HIV-infected patients will also be useful for the practitioner, as diagnostic tools for the prediction of the development of the viral disease. Specifically, it is goal to establish a correlation between:

the in vivo presence of such anti-idtiotypic antibodies in HIV-infected patients (specifically neutralizing antibodies) against the HIV particles, on one hand, and

the viral load and the disease progression on the other hand.

The screening methods used to identify these anti-idiotypic antibodies are well known by the one skill in the art, such as ELISA assays using as reagents, for example 5[Kψ (CH₂N)PR]-TASP proteins or fragments of interest.

In another embodiment of the therapeutic composition according to the invention, the said composition comprises a polynucleotide coding for the P95/nucleolin, P40/PHAPII and P30/PHAPI or one of its above-described peptide fragment or oligomeric peptide of pharmaceutical interest.

For the purpose of the present invention, a method of gene therapy consists in the in vivo production of a therapeutic peptide fragment or oligomeric peptide by the introduction of the genetic information in the HIV infected organism. This genetic information may be introduced in vitro in cell that has been previously extracted from the organism, the modified cell being subsequently reintroduced in the said organism, directly in vivo into the appropriate tissue. It is no need to say that the resultant recombinant protein or peptide will not constitute a functional target for HIV particles in vivo.

The method for delivering the corresponding protein or peptide to the interior of a cell of a vertebrate in vivo comprises the step of introducing a preparation comprising a pharmaceutically acceptable injectable carrier and a naked polynucleotide operatively coding for the polypeptide into the interstitial space of a tissue comprising the cell, whereby the naked polynucleotide is taken up into the interior of the cell and has a pharmaceutical effect.

In a specific embodiment, the invention provides a pharmaceutical product, comprising a naked polynucleotide operatively coding for the the P95/nucleolin, P40/PHAPII and P30/PHAPI or one of its above-described peptide fragment or oligomeric peptide, in solution in a physiologically acceptable injectable carrier and suitable for introduction interstitially into a tissue to cause cells of the tissue to express the said protein or polypeptide.

Advantageously, the therapeutic composition containing a complete or a part of the polynucleotide corresponding to the nucleic sequence of the P95/nucleolin, P40/PHAPII and P30/PHAPI or one of its above-described peptide fragment or oligomeric peptide is administered locally, near the site to be treated.

The polynucleotide operatively coding for the the P95/nucleolin, P40/PHAPII and P30/PHAPI or one of its above-described peptide fragment or oligomeric peptide may be a vector comprising the genomic DNA or the complementary DNA (cDNA) coding for the corresponding protein or its protein derivative and a promoter sequence allowing the expression of the genomic DNA or the complementary DNA in the desired eukaryotic cells, such as vertebrate cells, specifically mammalian cells.

The vector component of a therapeutic composition according to the present invention is advantageously a plasmid, a part of which is of viral or bacterial origin, which carries a viral or a bacterial origin of replication and a gene allowing its selection such as an antibiotic resistance gene.

By <<vector>> according to this specific embodiment of the invention is intended a circular or linear DNA molecule.

This vector may also contain an origin of replication that allows it to replicate in the eukaryotic host cell such as an origin of replication from a bovine papillomavirus.

The promoter carried by the said vector is advantageously the cytomegalovirus promoter (CMV). Nevertheless, the promoter may also be any other promoter with the proviso that the said promoter allow an efficient expression of the DNA insert coding for the the P95/nucleolin, P40/PHAPII and P30/PHAPI or one of its above-described peptide fragment or oligomeric peptide within the host.

Thus, the promoter is selected among the group comprising:

an internal or an endogenous promoter, such as the natural promoter associated with the structural gene coding for the P95/nucleolin, P40/PHAPII and P30/PHAPI or one of its above-described peptide fragment or oligomeric peptide; such a promoter may be completed by a regulatory element derived from the vertebrate host, in particular an activator element;

a promoter derived from a cytoskeletal protein gene such as the desmin promoter (Bolmont et al., J. of Submicroscopic cytology and pathology, 1990, 22:117-122; Zhenlin et al., Gene, 1989, 78:243-254).

As a general feature, the promoter may be heterologous to the vertebrate host, but it is advantageously homologous to the vertebrate host.

By a promoter heterologous to the vertebrate host is intended a promoter that is not found naturally in the vertebrate host.

Therapeutic compositions comprising a polynucleotide are described in the PCT application No WO 90/11092 (Vical Inc.) and also in the PCT application No WO 95/11307 (Institut Pasteur, INSERM, Université d'Ottawa) as well as in the articles of Tacson et al. (1996, Nature Medicine, 2(8):888-892) and of Huygen et al. (1996, Nature Medicine, 2(8):893-898).

In another embodiment, the DNA to be introduced is complexed with DEAE-dextran (Pagano et al., 1967, J. Virol., 1:891) or with nuclear proteins (Kaneda et al., 1989, Science, 243:375), with lipids (Feigner et al., 1987, Proc. Natl. Acad. Sci., 84:7413) or encapsulated within liposomes (Fraley et al., 1980, J. Biol. Chem., 255:10431).

In another embodiment, the therapeutic polynucleotide may be included in a transfection system comprising polypeptides that promote its penetration within the host cells as it is described in the PCT application WO 95/10534 (Seikagaku Corporation).

The therapeutic polynucleotide and vector according to the present invention may advantageously be administered in the form of a gel that facilitates their transfection into the cells. Such a gel composition may be a complex of poly-L-lysine and lactose, as described by Midoux (1993, Nucleic Acids Research, 21:871-878) or also poloxamer 407 as described by Pastore (1994, Circulation, 90:I-517). The therapeutic polynucleotide and vector according to the invention may also be suspended in a buffer solution or be associated with liposomes.

Thus, the therapeutic polynucleotide and vector according to the invention are used to make pharmaceutical compositions for delivering the DNA (genomic DNA or cDNA) coding for the P95/nucleolin, P40/PHAPII and P30/PHAPI protein or one of its biologically active derivatives at the site of the injection.

The amount of the vector to be injected vary according to the site of injection and also to the HIV load of the patient to be treated. As an indicative dose, it will be injected between 0, 1 and 100 μg of the vector in a patient.

In another embodiment of the therapeutic polynucleotide according to the invention, this polynucleotide may be introduced in vitro in a host cell, preferably in a host cell previously harvested from the patient to be treated and more preferably a somatic cell such as a muscle cell. Indeed the natural target cells of HIV are not used as recipient cells for the therapeutic nucleotide according to the present invention. In a subsequent step, the cell that has been transformed with the therapeutic nucleotide coding for the P95/nucleolin, P40/PHAPII and P30/PHAPI protein or one of its biologically active derivative is implanted back into the patient body in order to deliver the recombinant protein within the body either locally or systemically.

By biologically active derivative of the P95/nucleolin, P40/PHAPII or P30/PHAPI protein is meant one of their peptide or pseudopeptide counterparts or fragments.

Preferred DNA constructs used according to the gene therapy above described embodiments of the invention are proteins or peptides that are excerpted from the recombinant cell producing them, thus fusion peptide containing suitable signals in order to direct the protein towards the cell surface (such as signal peptide) and to secrete the mature recombinant protein or peptide out of the transfected/transformed producing cell.

In a prefered embodiment, gene targeting techniques are used for introducing a defect copy of a gene encoding either P95/nucleolin, P40/PHAPII and P30/PHAPI, in order to express a defect protein at the cell surface and thus destabilizing the V3 loop HIV receptor which will no long have the ability to bind the HIV retrovirus.

The defect copy of the gene coding for P95/nucleolin, P40/PHAPII or P30/PHAPI protein consists in one polynucleotide reported in FIG. 49 that has undergone a deletion, addition or substitution of one or several bases, preferably of 2 to 100 bases, more preferably 10 to 50 bases, such that the resultant encoded protein possess such conformational changes that the V3 loop HIV receptor is destabilized.

Another embodiment of a defect copy of the gene coding for P95/nucleolin, P40/PHAPII and P30/PHAPI protein is the insertion of a stop codon, preferably at a site near the 5′end of the coding sequence, in order to produce a truncated protein that is no long able to bind to the V3 loop of HIV gp120/gp125 and/or has the ability to destabilize the V3loop HIV repeceptor of the invention.

As a preferred embodiment, the gene targetting method comprises the introduction of a defect copy of two genes among the genes coding for P95/nucleolin, P40/PHAPII and P30/PHAPI protein and more preferably a defect copy of the three genes coding for P95/nucleolin, P40/PHAPII and P30/PHAPI protein. According to this specific embodiment, the two or three defect gene copies may be inserted in a single insertion vector or in separate insertion vectors, depending on the ability of the choosen vector to carry long heterologous protein encoding polynucleotides. As a more preferred ambodiment of the gene targetting method according to the present invention, a defect gene copy encoding a defect or truncated P95/nucleolin is always used, either alone or in combination with the other defect gene copies coding for P40/PHAPII and/or P30/PHAPI, as decribed above.

One of the prefered targetting techniques according to the present invention consists in a process for specific replacement, in particular by targeting the P95/nucleolin, P40/PHAPII and P30/PHAPI protein encoding DNA, called insertion DNA, comprising all or part of the DNA structurally encoding for the P95/nucleolin, P40/PHAPII and P30/PHAPI protein or one of its biologically active derivatives, when it is recombined with a complementing DNA in order to supply a complete recombinant gene in the genome of the host cell of the patient, characterized in that:

the site of insertion is located in a selected gene, called the recipient gene, containing the complementing DNA encoding the defect copy of P95/nucleolin, P40/PHAPII and P30/PHAPI protein or one of its biologically active derivatives and in that

the polynucleotide coding for the altered P95/nucleolin, P40/PHAPII and P30/PHAPI protein or one of its biologically active derivatives may comprise:

<<flanking sequences>> on either side of the DNA to be inserted, respectively homologous to two genomic sequences which are adjacent to the desired insertion site in the recipient gene.

the insertion DNA being heterologous with respect to the recipient gene, and

the flanking sequences being selected from those which constitute the above-mentioned complementing DNA and which allow, as a result of homologous recombination with corresponding sequences in the recipient gene, the reconstitution of a complete recombinant gene in the genome of the eukaryotic cell.

Such a DNA targetting technique is described in the PCT patent application No WO 90/11354 (Institut Pasteur).

Such a DNA targetting process makes it possible to insert the therapeutic nucleotide according to the invention behind an endogenous promoter which has the desired functions (for example, specificity of expression in the selected target tissue).

According to this embodiment of the invention, the inserted therapeutic polynucleotide may contain between the flanking sequences and upstream from the open reading frame encoding the P95/nucleolin, P40/PHAPII and P30/PHAPI protein or one of its biologically active derivatives, a sequence carrying a promoter sequence either homologous or heterologous with respect to the P95/nucleolin, P40/PHAPII and P30/PHAPI encoding DNA. The insertion DNA may contain in addition, downstream from the open reading frame and still between the flanking sequences, a gene coding for a selection agent, associated with a promoter making possible its expression in the target cell.

According to this embodiment of the present invention, the vector used contains in addition a bacterial origin of replication of the type colE1, pBR322, which makes the clonings and preparation in E. coli possible. A prefered vector is the plasmid pGN described in the PCT application No WO 90/11354.

Other gene therapy methods than those using homologous recombination may also be used in order to allow the expression of a polynucleotide encoding an altered copy of the P95/nucleolin, P40/PHAPII and P30/PHAPI protein or one of its biologically active derivatives within a patient's body.

In all the gene therapy methods that may be used according to the present invention, different types of vectors are utilized.

In one specific embodiment, the vector is derived from an adenovirus. Adenoviruses vectors that are suitable according to the gene therapy methods of the present invention are those described by Feldman and Steg (1996, Medecine/Sciences, synthese, 12:47-55) or Ohno et al. (1994, Sciences, 265:781-784) or also in the French patent application No FR-94.03.151 (Institut Pasteur, Inserm). Another prefered recombinant adenovirus according to this specific embodiment of the present invention is the adenovirus described by Ohwada et al. (1 996) or the human adenovirus type 2 or 5 (Ad 2 or Ad 5) or an adenovirus of animal origin (French patent application No FR-93.05954).

Among the adenoviruses of animal origin it can be cited the adenoviruses of canine (CA V2, strain Manhattan or A26/61[ATCC VR-800]), bovine, murine (Mav1, Beard et al., 1980, Virology, 75:81) or simian (SAV).

Preferably, the inventors are using recombinant defective adenoviruses that may be prepared following a technique well-known by one skill in the art, for example as described by Levrero et al., 1991, Gene, 101:195) or by Graham (1984, EMBO J., 3:2917) or in the European patent application No EP-185.573. Another defective recombinant adenovirus that may be used according to the present invention, as well as a pharmaceutical composition containing such a defective recombinant adenovirus, is described in the PCT application No. WO 95/14785.

A prefered retroviral vector used according to this specific embodiment of the present invention is derived from the Mo-MuL V retrovirus (WO 94/24298) or the retroviral vector described by Roth et al. (1996).

In another specific embodiment, the vector is a recombinant retroviral vector, such as the vector described in the PCT application No WO 92/15676 or the vector described in the PCT application No WO 94/24298 (Institut Pasteur). The latter recombinant retroviral vector comprises:

a DNA sequence from a provirus that has been modified such that:

the gag, pol and env genes of the provirus DNA has been deleted at least in part in order to obtain a proviral DNA which is incapable of replicate, this DNA not being able to recombine to form a wild virus;

the LTR sequence comprises a deletion in the U3 sequence, such that the mRNA transcription that the LTR controls is significantly reduced, for example at least 10 times, and

the retroviral vector comprises in addition an exogenous nucleotide sequence coding foran altered P95/nucleolin, P40/PHAPII and P30/PHAPI protein or one of its biologically active derivatives under the control of an exogenous promoter, for example a constitutive or an inductible promoter.

By exogenous promoter in the recombinant retroviral vector described above is intended a promoter that is exogenous with respect to the retroviral DNA but that may be endogenous or homologous with respect to the P95/nucleolin, P40/PHAPII and P30/PHAPI protein entire or partial nucleotide coding sequence.

In the case in which the promoter is heterologous with respect to the P95/nucleolin, P40/PHAPII and P30/PHAPI protein entire or partial nucleotide coding sequence, the promoter is preferably the mouse inductible promoter Mx or a promoter comprising a tetracyclin operator or also a hormone regulated promoter. A prefered constitutive promoter that is used is one of the internal promoters that are active in the resting fibroblasts such the promoter of the phosphoglycerate kinase gene (PGK-1). The PGK-1 promoter is either the mouse promoter or the human promoter such as described by Adra et al.(1987, Gene, 60:65-74). Other constitutive promoters may also be used such that the beta-actin promoter (Kort et al., 1983, Nucleic Acids Research, 11:8287-8301) or the vimentin promoter (Rettlez and Basenga, 1987, Mol. Cell. Biol., 7:1676-1685).

A prefered retroviral vector used according to this specific embodiment of the present invention is derived from the Mo-MuL V retrovirus (WO 94/24298).

In one prefered embodiment, the recombinant retroviral vector carrying the therapeutic nucleotide sequence coding for an altered P95/nucleolin, P40/PHAPII and P30/PHAPI protein or one of its biologically active derivatives is used to transform mammalian cells, preferably autologous cells from the mammalian host to be treated, and more preferably autologous fibroblasts from the patient to be treated. The fibroblasts that have been transformed with the retroviral vector according to the invention are reimplanted directly in the patient's body or are seeded in a preformed implant before the introduction of the implant colonized with the transformed fibroblasts within the patient's body. The implant used is advantageously made of a biocompatible carrier allowing the transformed fibroblasts to anchor associated with a compound allowing the gelification of the cells. The biocompatible carrier is either a biological carrier, such as coral or bone powder, or a synthetic carrier, such as synthetic polymer fibres, for example polytetrafluoroethylene fibres.

The therapeutic compositions described above may be administered to the vertebrate host by a local route such as an intramuscular route.

The therapeutic polynucleotide according to the present invention may be injected to the host after it has been coupled with compounds that promote the penetration of the therapeutic polynucleotide within the cell or its transport to the cell nucleus. The resulting conjugates may be encapsulated in polymer microparticles as it is described in the PCT application No. WO 94/27238 (Medisorb Technologies International).

Other therapeutic compositions according to the present invention comprise advantageously an oligonucleotide fragment of the nucleic sequence of P95/nucleolin, P40/PHAPII and P30/PHAPI of the invention (see FIG. 49 +L) as an antisense tool that inhibit the expression of the corresponding gene and is thus useful in order to destabilize the V3loop receptor of the invention and consequently prevent the binding of HIV to the cells. Preferred methods using antisense polynucleotide according to the present invention are the procedures described by Sczakiel et al. (1995).

Preferably, the antisense tools are choosen among the polynucleotides (15-200 bp long) that are complementary to the 5′end of the P95/nucleolin, P40/PHAPII or P30/PHAPI mRNA. Particularly, a combination of polynucleotides complementary to both P95/nucleolin, P40/PHAPII and P30/PHAPI mRNAs is used, specifically polynucleotides complementary to the 5′end of the latter mRNAs. In another embodiment, a combination of different antisense polynucleotides complementary to different parts of the desired targetted gene are used.

An alternative to the antisense technology that is used according to the present invention consists in using ribozymes that will bind to a target sequence via their complementary polynucleotide tail and that will cleave the corresponding RNA by hydrolyzing its target site (namely <<hammerhead ribozymes>>). Briefly, the simplified cycle of a hammerhead ribozyme consists of (1) sequence specific binding to the target RNA via complementary antisense sequences; (2) site-specific hydrolysis of the cleaveble motif of the target strand; and (3) release of cleavage products, which gives rise to another catalytic cycle. Indeed, the use of long-chain antisense polynucleotide (at least 30 bases long) or ribozymes with long antisense arms are advantageous. A preferred delivery system for antisense ribozyme is achieved by covalently linking these antisense ribozymes to lipophilic groups or to use liposomes as a convenient vector. Preferred antisense ribozymes according to the present invention are prepared as described by Sczakiel et al. (1995), the specific preparation procedures being referred to in said article being herein incorporated by reference.

Another method for inactivating the expression of the genes encoding the P95/nucleolin, P40/PHAPII and P30/PHAPI proteins according to the present invention is the use of the RNAse L. 2-5A-dependent Rnase is a latent endonuclease that requires the unusual 2′-5′-phosphodiester linked trimeric oligonucleotide ppp5′A2′p5′A2′p5′A for activation. The synthesis of chimeric molecules that link the antisense strategy with the 2-5A system provides specificity to the 2-5A-dependent Rnase that consequently can cleave specifically targetted sequences (Torrence et al., 1993; Maran et al., 1994). The use of this technique in combination with the antisense polynucleotides according to the invention is also part of the present invention.

Preferred antisense polynucleotides according to the present invention are complementary to a sequence of the mRNAs of P95/nucleolin, P40/PHAPII or P30/PHAPI that contains the translation initiation codon ATG. As an illustrative embodiment of such preferred antisense polynucleotides are 30 mer polynucleotides that are complementary to the following cDNA sequences:

a) P95/nucleolin: 5′-CGCCGCCATC ATGGTGAAGC TCGCGAAGGT-3′, which corresponds to the cDNA sequence beginning at nucleotide in position 1161 and ending at nucleotide in position 1190 of the nucleic sequence shown in FIG. 49 +L, Section II.

b) P30/PHAPI: 5′-GAGAGCGCGA GAGATGGAGA TGGGCAGACG-3′, which corresponds to the cDNA sequence beginning at nucleotide in position 91 and ending at nucleotide in position 120 of the nucleic sequence shown in FIG. 49 +L, Section III.

c) P40/PHAPII: 5′-GCAGCACCAT GTCGGCGCCG GCGGCCAAAG-3′, which corresponds to the cDNA sequence beginning at nucleotide in position 11 and ending at nucleotide in position 40 of the nucleic sequence shown in FIG. 49 +L, Section IV.

The observation that recombinant gp120 binds specifically and at a high affinity the purified preparation containing nucleolin/PHAP II/PHAP I provides a convenient assay for testing potential inhibitors that block such an interaction, and consequently virus infection. Consistent with this, the inventors have demonstrated here that inhibitors of HIV infection, such as the pseudopeptide 5[Kψ(CH₂N)PR]-TASP and antibodies specific for the V3 loop, block the interaction of the HIV envelope glycoprotein to nucleolin/PHAP II/PHAP I.

Thus, another subject of the present invention is a method for screening ligands that bind to the P95/nucleolin, P40/PHAPII or P30/PHAPI protein.

Such a screening method, in one embodiment, comprises the steps of:

a) Preparing a complex between the P95/nucleolin, P40/PHAPII or P30/PHAPI protein and a ligand that binds to the P95/nucleolin, P40/PHAPII or P30/PHAPI protein by bringing into contact the purified P95/nucleolin, P40/PHAPII or P30/PHAPI protein with a solution containing a molecule to be tested as a ligand binding to the P95/nucleolin, P40/PHAPII or P30/PHAPI protein;

b) visualizing the complex formed between the purified P95/nucleolin, P40/PHAPII or P30/PHAPI protein and the molecule to be tested.

The visualization of the complex formed between the purified P95/nucleolin, P40/PHAPII or P30/PHAPI protein and the molecule to be tested is done according to the conventional methods well known from the one skill in the art.

Specifically, the visualization consists in an ELISA assay wherein the purified natural or recombinant P95/nucleolin, P40/PHAPII or P30/PHAPI protein is immobilized, for example passively adsorbed, onto the surface of an ELISA microtiter plate (5 μg protein per well). Then, the coated wells are incubated with increasing concentrations of the peptidic or non peptidic candidate ligand to be tested (0.01 mM−10 mM) in a suitable buffer solution, for example overnight at 37° C. Subsequently, the wells are washed with a conventional ELISA washing buffer solution in order to eliminate the unbound candidate ligand molecules and then incubated with labeled V3 loop peptide or 5[Kψ (CH₂N)PR]-TASP (1 mM) or alternatively with labeled monoclonal or polyclonal antibodies directed to P95/nucleolin, P40/PHAPII or P30/PHAPI protein. The amount of labeling in each well is then measured and compared to positive and negative control wells, in order to determine the binding capacity of the candidate ligand molecule to the immobilized P95/nucleolin, P40/PHAPII or P30/PHAPI protein. The above labeled compounds are either radioactively or non radioactively labeled (biotin etc.).

For the purpose of the present invention, a ligand means a molecule, such as a protein, a peptide, an antibody or any synthetic chemical compound capable of binding to the P95/nucleolin, P40/PHAPII or P30/PHAPI protein or one of its biologically active derivatives or to modulate the expression of the polynucleotide coding for the P95/nucleolin, P40/PHAPII or P30/PHAPI protein or coding for one of its biologically active derivatives.

In the ligand screening method according to the present invention, a biological sample or a defined molecule to be tested as a putative ligand of the P95/nucleolin, P40/PHAPII or P30/PHAPI protein is brought into contact with the purified P95/nucleolin, P40/PHAPII or P30/PHAPI protein, for example the purified recombinant P95/nucleolin, P40/PHAPII or P30/PHAPI protein produced by a recombinant cell host as described hereinbefore, in order to form a complex between the P95/nucleolin, P40/PHAPII or P30/PHAPI protein and the putative ligand molecule to be tested.

In a particular embodiment of the screening method, the putative ligand is the expression product of a DNA insert contained in a phage vector (Parmley and Smith, Gene, 1988, 73:305-318). Specifically, random peptide phages libraries are used, the random DNA inserts being coding for peptides of 8 to 20 aminoacids in length (Oldenburg et al., 1992; Valadon et al., 1996; Lucas, 1994; Westerink et al., 1995; Castagnoli et al., 1991). According to this particular embodiment, the recombinant phages expressing a protein that binds to the immobilized P95/nucleolin, P40/PHAPII or P30/PHAPI protein is retained and the complex formed between the P95/nucleolin, P40/PHAPII or P30/PHAPI protein and the recombinant phage is subsequently immunoprecipitated by a polyclonal or a monoclonal antibody directed against the P95/nucleolin, P40/PHAPII or P30/PHAPI protein.

Once the ligand library in recombinant phages has been constructed, the phage population is brought into contact with the immobilized P95/nucleolin, P40/PHAPII or P30/PHAPI protein. Then the preparation of complexes is washed in order to remove the non-specifically bound recombinant phages. The phages that bind specifically to the P95/nucleolin, P40/PHAPII or P30/PHAPI protein are then eluted by a buffer (acid pH) or immunoprecipitated by the monoclonal antibody produced by the hybridoma anti-P95/nucleolin, P40/PHAPII or P30/PHAPI, and this phage population is subsequently amplified by an over-infection of bacteria (for example E. coli). The selection step may be repeated several times, preferably 2-4 times, in order to select the more specific recombinant phage clones. The last step consists in characterizing the peptide produced by the selected recombinant phage clones either by expression in infected bacteria and isolation, expressing the phage insert in another host-vector system, or sequencing the insert contained in the selected recombinant phages.

Another subject of the present invention is a method for screening molecules that modulate the expression of the P95/nucleolin, P40/PHAPII or P30/PHAPI protein. Such a screening method comprises the steps of:

a) cultivating a prokaryotic or an eukaryotic cell that has been transfected with a nucleotide sequence encoding the P95/nucleolin, P40/PHAPII or P30/PHAPI protein, placed under the control of its own promoter,

b) bringing into contact the cultivated cell with a molecule to be tested;

c) quantifying the expression of the P95/nucleolin, P40/PHAPII or P30/PHAPI protein.

Using DNA recombination techniques well known by the one skill in the art, the P95/nucleolin, P40/PHAPII or P30/PHAPI protein encoding DNA sequence is inserted into an expression vector, downstream from its promoter sequence. As an illustrative example, the promoter sequence of the P95/nucleolin gene is contained in the nucleic sequence presented in FIG. 49 +L, Section II.

The quantification of the expression of the P95/nucleolin, P40/PHAPII or P30/PHAPI protein may be realized either at the mRNA level or at the protein level. In the latter case, polyclonal or monoclonal antibodies may be used to quantify the amounts of the P95/nucleolin, P40/PHAPII or P30/PHAPI protein that have been produced, for example in an ELISA or a RIA assay.

In a prefered embodiment, the quantification of the P95/nucleolin, P40/PHAPII or P30/PHAPI mRNA is realized by a quantitative PCR amplification of the cDNA obtained by a reverse transcription of the total mRNA of the cultivated P95/nucleolin, P40/PHAPII or P30/PHAPI-transfected host cell, using a pair of primers specific for P95/nucleolin, P40/PHAPII or P30/PHAPI.

As an illustrative example, a pair of primers used to quantitate P95/nucleolin, P40/PHAPII or P30/PHAPI reverse-transcribed mRNA is the following:

a) P95/nucleolin

Sense primer: 5′-CTTCGGGTGTACGTGCTCCGGG-3′, which is complementary to a sequence beginning at the nucleotide in position nt 1070 and ending at the nucleotide in position 1091 of the nucleic sequence reported in FIG. 49 +L, Section II.

Antisense primer: 5′-CCTGAGTGACTTTGTAAGGGAG-3′, which corresponds to a sequence beginning at the nucleotide in position nt 7069 and ending at the nucleotide in position nt 7090 of the nucleic sequence reported in FIG. 49 +L, Section II.

Specific probe: a polynucleotide having the nucleic sequence of the amplicon itself.

b) P30/PHAPI

Sense primer: 5′-CCGCCGGCGCGGCAGCCTCTG-3′, which is complementary to a sequence of the nucleic sequence reported in FIG. 49 +L, Section III.

Antisense primer: 5′-GTCATCATCTTCTCCCTCATC-3′, which corresponds to a nucleic sequence of the nucleic sequence reported in 49 +L, Section III.

Specific probe: a polynucleotide having the nucleic sequence of the amplicon itself.

c0 P40/PHAPII

Sense primer: 5′-CGACCGCGGAGCAGCACCATG-3′, which is complementary to a sequence of the nucleic sequence reported in FIG. 49 +L, Section IV.

Antisense primer: 5′-GGAAGGTTGGAATCCATCAG-3′, which corresponds to a sequence of the nucleic sequence reported in FIG. 49 +L, Section IV.

Specific probe: a polynucleotide having the nucleic sequence of the amplicon itself.

The process for determining the quantity of the cDNA corresponding to the P95/nucleolin, P40/PHAPII or P30/PHAPI mRNA present in the cultivated P95/nucleolin, P40/PHAPII or P30/PHAPI-transfected cells is characterized in that:

1) a standard DNA fragment, which differs from the P95/nucleolin, P40/PHAPII or P30/PHAPI cDNA fragment, obtained by the reverse transcription of the P95/nucleolin, P40/PHAPII or P30/PHAPI-mRNA, but can be amplified with the same oligonucleotide primers is added to the sample to be analyzed containing the P95/nucleolin, P40/PHAPII or P30/PHAPI-cDNA fragment, the standard DNA fragment and the P95/nucleolin, P40/PHAPII or P30/PHAPI-cDNA fragment differing in sequence and/or size by not more than approximately 10%, and preferably by not more than 5 nucleotides by strand,

2) the P95/nucleolin, P40/PHAPII or P30/PHAPI-cDNA fragment and the standard DNA fragment are coamplified with the same oligonucleotide primers, preferably to saturation of the amplification of the P95/nucleolin, P40/PHAPII or P30/PHAPI-cDNA fragment,

3) to the reaction medium obtained in step 2), there are added:

either two types of labeled oligonucleotide probes which are each specific for the P95/nucleolin, P40/PHAPII or P30/PHAPI-cDNA fragment ant the standard DNA fragment, respectively, and different from the amplification oligonucleotide primers of step2),

or one or more labeled oligonucleotide primer(s), specific for the P95/nucleolin, P40/PHAPII or P30/PHAPI-cDNA fragment and the standard DNA fragment and different from said oligonucleotide primers of step 2), and one or more additional amplification cycle(s) with said labeled oligonucleotide primer(s) is/are performed, so that, during a cycle, after denaturation of the DNA, said labeled oligonucleotide primer(s) hybridize(s) with said fragments at a suitable site in order that an elongation with the DNA polymerase generates labeled DNA fragments of different sizes and/or sequences and/or with different labels according to wether they originate from the DNA fragment of interest or the standard fragment, respectively, and then

4) the initial quantity of P95/nucleolin, P40/PHAPII or P30/PHAPI-cDNA fragment is determined as being the product of the initial quantity of standard DNA fragment and the ratio of the quantity of amplified P95/nucleolin, P40/PHAPII or P30/PHAPI-cDNA fragment, which ratio is identical to that of the quantities of the labeled DNA fragments originating from the amplified P95/nucleolin, P40/PHAPII or P30/PHAPI-cDNA fragment, respectively, obtained in step 3).

More technical details regarding the performing of the quantitaive PCR amplification reaction are found in the PCT application No WO 93/10257 (Institut Pasteur, Inserm), the specific technical teachings contained in this PCT application beeing herein incorporated by reference.

A further object of the present invention consists in therapeutic compositions comprising a therapeutic molecule that is able to modify the interaction between, on one hand the V3 loop HIV receptor of the invention present at the cell surface of a patient infected with a human HIV retrovirus, specifically HIV-1 or HIV-2, and on the other hand the gp120/gp125 envelope glycoproteins of said HIV retroviruses.

Such therapeutic molecule according to the present invention is choosen among the followings:

a) The purified P95/nucleolin, P40/PHAPII or P30/PHAPI protein or at least one of their peptide or pseudopeptide fragments, either under a monomeric or polymeric form, including the peptides or pseudopeptides presented as MAP constructs;

b) A monoclonal or polyclonal antibody directed to the P95/nucleolin, P40/PHAPII or P30/PHAPI protein;

c) A purified anti-idiotypic antibody mimmicking the P95/nucleolin, P40/PHAPII or P30/PHAPI protein;

d) A peptidic or non peptidic ligand molecule binding specifically to the P95/nucleolin, P40/PHAPII or P30/PHAPI protein which has been selected according to one of the screening methods described above;

e) An antisense polynucleotide, optionally linked to a ribozyme or to a Rnase, specifically the 2-5A-dependent Rnase, as described above.

f) A therapeutic polynucleotide coding for the P95/nucleolin, P40/PHAPII or P30/PHAPI protein or one of its biologically active derivatives, optionally carried by an expression vector,

g) A therapeutic polynucleotide consisting in a defect gene copy of the P95/nucleolin, P40/PHAPII or P30/PHAPI protein, carried by an insertion vector.

The therapeutic compositions according to the present invention are administered to the patient systemically or by a local route.

In a preferred embodiment, the therapeutic compositions are administered via a systemic route, i.e. by an intra-venous injection.

The present inventors have determined that the 5[Kψ (CH₂N)PR]-TASP construct has a total inoccuity in the adult rat, even at an amount of 3 mg/kg.

As already mentioned, the inventors have shown that 100% saturation of the binding sites are obtained with the following concentrations of the 5[Kψ (CH₂N)PR]-TASP construct, mimmicking the V3 loop of the HIV gp120 glycoprotein:

2 μM of 5[Kψ (CH₂N)PR]-TASP for P95/nucleolin;

4 μM of 5[Kψ (CH₂N)PR]-TASP for P40/PHAPII; and

8 μM of 5[Kψ (CH₂N)PR]-TASP for P30/PHAPI.

Consequently, the therapeutic compositions comprising the the P95/nucleolin, P40/PHAPI or P30/PHAPI or one of its above-described peptide fragment or oligomeric peptide or their pseudopeptide counterparts, as well as the peptidic or non peptidic selected ligand molecules, are advantageously administered to the patient at an amount per body weight in the range corresponding to an equivalent pharmaceutically effective amount of 5[Kψ (CH₂N)PR]-TASP, preferably in the range between 0.1 and 5 mg of 5[Kψ (CH₂N)PR]-TASP equivalent pharmaceutically active amount.

The equivalent pharmaceutically effective amounts of the above-defined therapeutic molecules is determined by measuring the amount of the therapeutic molecule which is necessary to saturate 100% of the P95/nucleolin, P40/PHAPII or P30/PHAPI sites.

The amount of the antisense or of the therapeutic polynucleotide according to the invention to be administered to a patient is either already described above in the specification or can be found in the corresponding above-cited litterature.

In a specific embodiment of the therapeutic compositions according to the present invention, the therapeutic molecules of the invention are combined with other anti-HIV molecules, such as protease inhibitors, or modified nuleotides such as AZT or DDI.

Indeed, the inventors have shown a synergistic action between the 5[Kψ (CH₂N)PR]-TASP construct and AZT again an HIV infection. Consequently, a pharmaceutical composition containing a combination of at least AZT and a therapeutic molecule according to the present invention is also part of the present invention.

The therapeutic molecules may also be combined with other anti-HIV compounds, such as chemokines like Rantes, SDF-1, MIP-1α or MIP-1β. These chemokines may be presented either under their natural form or under a modified form such that their ability to bind to their respective receptor is preserved whereas their chmoattractive biological activity is lost. Such a modified SDF-1 chemokine is described in the PCT Application no WO98/04698 (Virelizier et al.). The therapeutic compositions according to the invention may be advantadgeously administered to patients that are infected by HIV isolates that have become resistant to other anti-HIV drugs such as modified nucleotides (like AZT or DDI), protease inhibitors (like Saquinavir) or also nonnucleotide reverse transcriptase inhibitors (like Neviparine).

Advantageously, the therapeutic compositions according to the present invention have a long half-life within the body.

A method for assessing the pharmacokinetics of the ligand molecules selected according to the invention consists in measuring the plasma clearance of the selected ligand molecules which is determined according to the technique described by Wu et al. in 1996 or the technique described by Ezan et al. in 1986, or by Ezan et al., 1996, which techniques are herein incorporated by reference.

The therapeutic compositions containing the P95/nucleolin, P40/PHAPII or P30/PHAPI protein or their biologically active derivatives and used according to the present invention may be either under the form of a liquid solution, under the form of a gel or under the form of a dry powder.

Such therapeutic compositions may be in the form of a saline solution or a tablet, preferably a controlled release tablet. A typical controlled release tablet is decribed in the PCT Patent Application No. WO 9622768, which contains from about 30 to about 70 percent by weight of one or more cellulose ethers such as hydroxypropyl methylcellulose, and from about 30 to about 70 percent by weight of an inert substance such as cornstarch.

In another embodiment of the therapeutic compositions of the present invention, the P95/nucleolin, P40/PHAPII or P30/PHAPI protein or their biologically active derivatives are included in a controlled release device to be placed locally in the body, in order to obtain a sustained delivery of the active molecules in the surrounding of the site to be treated.

Preferably, the controlled release devices that are used for the purpose of the present invention are lipid or polymer microparticles that dissolves or are hydrolyzed slowly within the body, specifically in the stomach or in the gastro-intestinal tract.

In a preferred embodiment of the controlled release devices of the present invention, the latters can be implanted locally in order to ensure a limited area diffusion of the active molecule, surrounding the organ or tissue to be treated.

Preferred sustained delivery devices according to the present invention contain biodegradable polymers such as described in the PCT Patent Application No. WO 9701331. The polymer may be a polysaccharide as in the PCT Patent Application No. WO 9613253, such as sodium alignate. A biodegradable sustained preparation is preferably composed of a polysaccharide which is coated with cationic molecules such as chitosan, the carrier being slowly enzymatically hydrolyzed, for example by lysozyme, in vivo after the release of the active molecule.

The polymer used in the controlled release devices according to the present invention may also be a polyvinylpyrrolidone type polymer, such as described in the PCT Patent Application No. WO 8804922 or a starch hydrolysate, such as described in the PCT Patent Application No. WO 9417676.

In a specific embodiment, the polymer is a bioadhesive polymer such as carboxymethylcellulose, Carbopol™, Polycarbophil™ or sodium alginate, that bind with an excellent efficiency to the mucin present at the surface of the epithelium (Robinson et al., 1988), these polymers being used especially in the case of an oral drug delivery.

Other preferred sustained delivery devices according to the present invention are under the form of polymer microbeads, for example porous crosslinked polymeric microbeads, such as described in the PCT Patent Application No WO 9533553.

Another embodiment of the controlled release devices according to the present invention are liposomes either in a hydrated form, such as in the PCT Patent Application No. WO 8601102 or in the PCT Patent Application No. WO 9522961 (Capron et al.), or in a dehydrated form, such as in the PCT Patent Application No. WO 8601103. Other lipid emulsions used as drug delivery systems that may be used for the purpose of the present invention are described by Davis et al. in 1988, that may be administered via the oral, parenteral or the intravenous route. The liposomes may contain saccharide determinants that bind to specific cell membrane components in order to facilitate the delivery of the active molecule towards a selected target cell, in particular saccahride determinants that bind to specific lectins of the cell membrane (Shen, 1988).

Another embodiment of the sustained delivery formulations used according to the present invention consists in a particle vector comprising, from the inner layer to the outer layer:

a non liquid hydrophilic core, for example a crosslinked polysaccharide or oligosaccharide matrix, said core being optionally grafted with ionic ligands carrying at least un group selected from phosphate, sulfate, carboxylic acid, quaternary ammonium, secondary amine or tertiary amine.

an external layer consisting in lipid compounds that are grafted onto the core by covalent bounds.

Such a particulate vector is described in the PCT Patent Application No. WO 94/23701 (Perrin et al.).

Prior art works have shown that the chemokine receptor CCR-5 serves as a cofactor of CD4 for the fusion and entry mediated monotropic HIV-1 isolates. Some individuals who resist HIV infection, and individuals who remain uninfected with HIV-1, despite multiple high risk sexual exposures, have been shown to express a deleted version of this cofactor CCR-5 (Liu et al., 1996; Dean et al., 1996). It should be noted that only a small proportion (about 3%) of the HIV-1 negative cohort shows the mutation in the CCR-5 receptor. Therefore, defects in other parameters implicated in HIV infection are probably responsible for the resistance of HIV negative individuals.

In view of the prior art reports, it is important to investigate in different cohorts, representing individual who resist HIV infection, wether the V3 loop receptor, i.e. P95/nucleolin, P40/PHAPII or P30/PHAPI complex, is functional. The lack of the P95/nucleolin, P40/PHAPII or P30/PHAPI complex, or the lack of anyone of the proteins in the complex, or any changes in the structure of the complex, or any change in any one of the proteins in the complex, will affect virus binding and infection. Such modifications might exist in HIV resistant individuals and in individuals who remain uninfected despite multiple high-risk sexual exposure.

Thus, methods of screening for the presence of a wild form of the V3 loop receptor of the invention as well as methods of screening for mutations in the V3 loop HIV receptor in HIV resistant individuals are a further object of the present invention.

A specific embodiment of the method for screening the normal expression of the V3 loop HIV receptor according to the invention consists in the use of monoclonal or polyclonal antibodies directed either to the whole receptor or to the P95/nucleolin, P40/PHAPII or P30/PHAPI protein on isolated patient cells, specifically peripheral blood mononuclear cells (PBMC), said antibodies being optionally radioactively or non radioactively labeled, and in the further detection of the bound antibodies onto said patients cells. A preferred method of visualization of the cell bound antibodies is the use of a Fluorescence Activated Cell Sorter apparatus.

A method of screening for mutations occurring either in the genes coding for P95/nucleolin, P40/PHAPII or P30/PHAPI protein comprises preferably the procedures described by Huang et al. (1996) and Samson et al. (1996) that have been used in order to dermine the genetic defects occuring in the CCR-5 gene.

Briefly, the full coding region of P95/nucleolin, P40/PHAPII or P30/PHAPI form HIV resisant patients is amplified using a pair of specific primers, the sequence of which is determined on the basis of the nucleic sequences reported in 49 +L49. The amplified DNA is then sequenced and differences between the wild genes (49 +L) and the amplified DNA of the HIV resistant patients.

The sequence differences between the defective P95/nucleolin, P40/PHAPII or P30/PHAPI genes and their wild counterparts allow to design specific oligonucleotides probes complementary to the mutated region(s) that are useful for diagnosis of a causal relationship between an HIV resistance phenotype and an alteration in the expression of the V3 loop HIV receptor of the invention.

Another method for identifying mutations occuring at the level of the P95/nucleolin, P40/PHAPII or P30/PHAPI genes are, for example, the FAMA technique described by Meo et al. (PCT application No WO 95/07361), which technique allox the determination of the mutation positions and which is herein incorporated by reference.

Finally, the present invention concerns a method for screening mutations occurring in the P95/nucleolin, P40/PHAPII or P30/PHAPI encoding genes, in order to make useful diagnostic tools suitable to adapt a specific therapy for HIV infected patients.

Thus, another object of the present invention consists in a method for detecting a genetic abnormality in P95/nucleolin, P40/PHAPII or P30/PHAPI in a biological sample containing DNA or cDNA, comprising the steps of:

a) bringing the biological sample into contact with a pair of oligonucleotide fragments according to the invention, the DNA or cDNA contained in the sample having been optionally made available to hybridization and under conditions permitting a hybridization of the said oligonucleotide fragments with the nucleic acid contained in the biological sample;

b) amplifying the DNA

c) revealing the amplification products;

d) optionally detecting a mutation or a deletion by appropriate techniques.

The step d) of the above-described method may consist in a Single-Starnd Polymorphism technique (SSCP), a Denaturing Gradient Gel Electrophoresis (DGGE), or the FAMA technique described in the PCT patent application No WO-95/07361.

Another object of the present invention consists in a method for detecting a genetic abnormality in P95/nucleolin, P40/PHAPII or P30/PHAPI in a biological sample containing DNA, or cDNA, comprising the steps of:

a) bringing the biological sample into contact with an oligonucleotide probe according to the invention, the DNA, mRNA or cDNA contained in the sample having been optionally made available to hybridization and under conditions permitting a hybridization of the primers with the nucleic acid contained in the biological sample;

b) detecting the hybrid formed between the oligonucleotide probe and the DNA conatained in the biological sample.

The present invention consists also in a method for detecting a genetic abnormality in P95/nucleolin, P40/PHAPII or P30/PHAPI in a biological sample containing DNA, comprising the steps of:

a) bringing into contact a first oligonucleotide probe according to the invention that has been immobilized on a support, the DNA contained in the sample having been optionally made available to hybridization and under conditions permitting a hybridization of the primers with the DNA contained in the biological sample;

b) bringing into contact the hybrid formed between the immobilized first oligonucleotide probe and the DNA contained in the biological sample with a second oligonucleotide probe according to the invention, which second probe hybridizes with a sequence different from the sequence to which the immobilized first probe hybridizes, optionally after having removed the DNA contained in the biological sample which has not hybridized with the immobilized first oligonucleotide probe.

Another object of the present invention consists in a method for detecting a genetic abnormality in P95/nucleolin, P40/PHAPII or P30/PHAPI in a biological sample containing DNA, by the detection of the presence and of the position of base substitutions or base deletions in a nucleotide sequence included in a double stranded DNA preparation to be tested, the said method comprising the steps of:

a) amplifying specifically the region containing, on one hand, the nucleotide sequence of the DNA to be tested and on the other hand the nucleotide sequence of a DNA of known sequence, the DNA of known sequence being a polynucleotide according to the invention;

b) labeling the sense and antisense strands of these DNA with diferent fluorescent or other non-isotopic labels;

c) hybridizing the amplified DNAs;

d) revealing the heteroduplex formed between the DNA of known sequence and the DNA to be tested by cleavage of the mismatched parts of the DNA strands

Such a mismatch localization technique has been described by Meo et al. in the PCT application No. WO-95/07361.

The invention also pertains to a kit for the detection of a genetic abnormality in P95/nucleolin, P40/PHAPII or P30/PHAPI in a biological sample, comprising the following elements:

a) a pair of oligonucleotides according to the invention;

b) the reagents necessary for carrying out a DNA amplification;

c)a component which makes it possible to determine the length of the amplified fragments or to detect a mutation.

Thus, is also part of the present invention a diagnostic method for detecting mutations in the gene coding for P95/nucleolin, P40/PHAPII or P30/PHAPI comprising the steps of:

a) amplifying the full coding region of P95/nucleolin, P40/PHAPII or P30/PHAPI from a patient using a pair of specific primers;

b) determining the sequence of the amplified DNA;

c) comparing the sequence obtained at step b) with the nucleic sequences of P95/nucleolin, P40/PHAPII or P30/PHAPI reported in 49 +L.

The present invention is also directed to a diagnostic nucleic probe comprising at least 20 nucleotides of a mutated sequence of P95/nucleolin, P40/PHAPII or P30/PHAPI, said probe containing at least one specific mutation identified according to the above-described method.

Nucleic probes according to the present invention, as described above, are specific to detect a genetic defect in one gene among the P95/nucleolin, P40/PHAPII or P30/PHAPI. These specific probes hybridize with the said mutated gene and does not hybridize with either the wild gene sequences reported in Annex 1 or with unraleted genes or sequences. Preferred oligonucleotide probes according to the invention are at least 20 nucleotides in length, and more preferably a length comprised between 20 and 300 nucleotides.

These specific diagnostic probes according to the present invention are used in high stringency hybridization conditions.

As an illustrative embodiment, the stringent hybridization conditions used in order to specifically detect a gene defect according to the present invention are advantageously the followings:

The hybridization step is realized at 65° C. in the presence of 6×SSC buffer, 5×Denhardt's solution, 0,5% SDS and 100 μg/ml of salmon sperm DNA.

The hybridization step is followed by four washing steps:

two washings during 5 min, preferably at 65° C. in a 2×SSC and 0.1% SDS buffer;

one washing during 30 min, preferably at 65° C. in a 2×SSC and 0.1% SDS buffer,

one washnig during 10 min, preferably at 65° C. in a 0.1×SSC and 0.1% SDS buffer.

The non-labeled polynucleotides or oligonucleotides of the invention may be directly used as probes. Nevertheless, the polynucleotides or oligonucleotides are generally labeled with a radioactive element (³²P, ³⁵S, ³H, ¹²⁵I) or by a non-isotopic molecule (for example, biotin, acetylaminofluorene, digoxigenin, 5-bromodesoxyuridin, fluorescein) in order to generate probes that are useful for numerous applications.

Examples of non-radioactive labeling of nucleic acid fragments are described in the french patent No FR-7810975 or by Urdea et al. or Sanchez-Pescador et al., 1988.

In the latter case, other labeling techniques may be also used such those described in the french patents FR-2,422,956 and 2,518,755. The hybridization step may be performed in diffrent ways (Matthews et al., 1988). The more general method consists in immobilizing the nucleic acid that has been extracted from the biological sample on a substrate (nitrocellulose, nylon, polystyren) and then to incubate, in defined conditions, the target nucleic acid with the probe. Subsequently to the hybridization step, the excess amount of the specific probe is discarded and the hybrid molecules formed are detected by an appropriate method (radioactivity, fluorescence or enzyme activity measurement).

Advantageously, the probes according to the present invention may have structural characteristics such that they allow the signal amplification, such structural characteristics beeing, for example, branched DNA probes as those described by Urdea et al. in 1991 or in the European patent No EP-0225,807 (Chiron).

In another advantageous embodiment of the probes according to the present invention, the latters may be used as <<capture probes>>, and are for this purpose immobilized on a substrate in order to capture the targer nucleic acid contained in a biological sample. The captured target nucleic acid is subsequently detected with a second probe which recognizes a sequence of the target nucleic acid which is different from the sequence recognized by the capture probe.

Another appropriate preparation process of the nucleic acids of the invention containing at most 200 nucleotides (or 200 bp if these molecules are double stranded) comprises the following steps:

synthesising DNA using the automated method of beta-cyanethylphosphoramidite described in 1986;

cloning the thus obtained nucleic acids in an appropriate vector;

purifying the nucleic acid by hybridizing an appropriate probe according to the present invention.

A chemical method for producing the nucleic acids according to the invention which have a length of more thant 200 nucleotides nucleotides (or 200 bp if these molecules are double stranded) comprises the following steps:

assembling the chemically synthesised oligonucleotides, having different restriction sites at each end.

cloning the thus obtained nucleic acids in an appropriate vector.

purifying the nucleic acid by hybridizing an appropriate probe according to the present invention.

The oligonucleotide probes according to the present invention may also be used in a detection device comprising a matrix library of probes immobilized on a substrate, the sequence of each probe of a given length being localized in a shift of one or several bases, one from the other, each probe of the matrix library thus being complementary of a distinct sequence of the target nucleic acid. Optionally, the substrate of the matrix may be a material able to act as an electron donnor, the detection of the matrix positions in which an hybridization has occurred being subsequently determined by an electronic device. Such matrix libraries of probes and methods of specific detection of a targer nucleic acid is described in the European patent application No EP-0713,016 (Affymax technologies) and also in the U.S. Pat. No. 5,202,231 (Drmanac).

An oligonucleotide probe matrix may advantadgeously be used to detect mutations occurring in P95/nucleolin, P40/PHAPII or P30/PHAPI gene. For this particular purpose, probes are specifically designed to have a nucleotidic sequence allowing their hybridization to the genes that carry known mutations (either by deletion, insertion of substitution of one or several nucleotides). By known mutations is meant mutations on the P95/nucleolin, P40/PHAPII or P30/PHAPI gene that have been identified according, for example to the technique used by Huang et al. (1996) or Samson et al. (1996).

Another technique that is used to detect mutations in the P95/nucleolin, P40/PHAPII or P30/PHAPI gene is the use of a high-density DNA array. Each oligonucleotide probe constituting a unit element of the high density DNA array is designed to match a specific subsequence of the P95/nucleolin, P40/PHAPII or P30/PHAPI genomic DNA or cDNA. Thus, an array consisting of oligonucleotides complementary to subsequences of the target gene sequence is used to determine the identity of the target sequence with the wild gene sequence, measure its amount, and detect differences between the target sequence and the reference wild gene sequence of the P95/nucleolin, P40/PHAPHU or P30/PHAPI gene, In one such design, termed 4L tiled array, is implemented a set of four probes (A, C, G, T), preferably I5-nucleotide oligomers. In each set of four probes, the perfect complement will hybridize more strongly than mismatched probes. Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a <<footprint>> for the probes flanking a mutation position. This technique was decribed by Chee et al. in 1996, which is herein incorporated by reference.

The present invention is further illustrated by the following Figures and

Examples, without in anyway being limited in scope to the specific embodiments described in said Figures and Examples.

### FIGURES

FIG. 1 +L. The effect of 5[Kψ(CH₂N)PR]-TASP on the binding of gp120 or HIV particles to CD4⁺ cells.

A. The binding of ¹²⁵I-labeled gp120 to CEM cells. Cells were preincubated (37° C.; 15 min) in the absence (column C) or presence of different concentrations of 5[Kψ(CH₂N)PR]-TASP (20, 40, 80 μM; indicated as TASP) or mAb OKT4A (2 mg/ml) before the addition of ¹²⁵I-labeled gp120 and further incubation for 1 hour. The cells were then washed as described in “Materials and Methods” and processed for analysis of the bound gp120. The 100% binding (column C) represents the value obtained in the absence of 5[Kψ(CH₂N)PR]-TASP.

B. The binding of HIV particles to CEM cells. CEM cells were preincubated in the absence (column C) or presence of 5[Kψ(CH₂N)PR]-TASP (10 μM; column TASP), mAb OKT4A (10 μg/ml; column OKT4A), and 5[Kψ(CH₂N)PR]-TASP+mAb OKT4A (10 μM and 10 μg/ml, respectively) before the addition of HIV-1. The binding of HIV particles was estimated as described in “Materials and Methods”. The ordinate gives the concentration of p24 associated with the cells, i.e. particles bound on the cell surface as well as particles (or cores) entered into cells. Each value represents the mean of two identical samples. Similar results were obtained in two other indepenent experiments.

Note: at these concentrations of 5[Kψ(CH₂N)PR]-TASP (10 μM) and mAb OKT4A (10 mg/ml), there was a complete inhibition of virus infection.

FIG. 2 +L. Specific binding of 5[Kψ(CH₂N)PR]-TASP to the cell surface.

The FITC-labeled 5[Kψ(CH₂N)PR]-TASP (referred here as P19*) at 0.5 μM was added in cultures of different cell lines, CEM (sections 1, 3, and 4), MOLT4 (section 2), and Daudi (section 5), or on the third day of PHA-stimulated PBMC (section 6), in the absence or presence of 50 μM unlabeled constructs as it is indicated: 5[Kψ(CH₂N)PR]-TASP (referred to as P19), 5[KGQ]-TASP (referred to as P18) and 5[KPR]-TASP referred to as P1). The fluorescence intensity was monitored by FACS analysis. The peak C gives the autofluorescence of each cell type incubated with unlabeled 0.5 mM 5[Kψ(CH₂N)PR]-TASP. The ordinates give the relative cell number, whereas the abscissa give the relative fluorescence intensity.

Note: all the different anti-HIV TASP constructs that manifested inhibitory activity on HIV infection (described in reference 7), could prevent the binding of FITC-labeled 5[Kψ(CH₂N)PR]-TASP to cells when added in excess (at 50 to 100 μM concentrations).

FIG. 3 +L. The peptide-TASP inhibitor binds to a cell-surface protein resistant to trypsin but sensitive to proteinase K digestion.

MOLT4 cells were treated as described in “Materials and Methods” with trypsin (2.5 mg/mL 5 min at 20° C.) or protease K (0.2 mg/ml, 30 min at 37° C.) before FACS analysis using the FITC-labeled 5[Kψ(CH₂N)PR]-TASP (p19*) and monoclonal antibodies specific for cell-surface proteins: mAb Ta1 against CD26 and mAbs OKT4 and OKT4A against CD4. The peak C in each section represents the corresponding control peak obtained by PE-labeled control mAb B4 (specific to CD19) for mAb Ta1, FITC-labeled MCG1 control antibody for mAbs OKT4 and OKT4A, and 0.5 μM unlabeled 5[Kψ(CH₂N)PR]-TASP for p19*.

FIG. 4 +L. The high affinity of 5[Kψ(CH₂N)PR]-TASP to bind its cell-surface ligand. CEM cells were analyzed by FACS analysis using biotin-labeled 5[KPR]-TASP (at 1, 5 and 10 μM), 5[Kψ(CH₂N)PR]-TASP (at 0.25, 0.5, 1 and 5 μM) and control 5[QPQ]- and 5[KGQ]-TASP (at 20 μM) as described in “Materials and Methods”. The peak C-gives the fluorescence of cells incubated with the unlabeled respective TASP constructs (20 mM). The ordinates give the relative cell number, whereas the abscissa give the relative fluorescence intensity.

FIG. 5 +L. The specific binding of 5[Kψ(CH₂N)PR]-TASP to a 95 kDa protein.

Crude CEM cell extracts (material corresponding to 10⁶ cells) were analyzed by ligand blotting using biotin-labeled 5[Kψ(CH₂N)PR]-TASP (“Materials and Methods”). To show the specificity of binding, the electrophoretic blots after saturation with the blocking buffer, were first incubated (4° C., 30 min) with 50 mM of unlabeled 5[KGQ]-TASP (section A) or 5[Kψ(CH₂N)PR]-TASP (section B) before the addition of biotin-labeled 5[Kψ(CH₂N)PR]-TASP (5 mM). The numbers in the middle (200, 97, 68 and 43) show the position of molecular weight (in kDa) protein markers.

Note: Ligand binding studies shown here and in FIGS. 6 and 7 were performed on reducing gels. It should be noted however, that similar results were obtained on non-reducing gels, i.e., in the absence of b-mercaptoethanol.

FIG. 6 +L. Isolation of cell-surface P95 complexed to 5[Kψ(CH₂N)PR]-TASP.

Lanes 2 to 5: CEM cells were washed and incubated at 4° C. for 30 min in FACS buffer (which contains sodium azide) with biotin-labeled control 5[KGQ]- or 5[QPQ]-TASP and anti-HIV 5[KPR]- or 5[Kψ(CH₂N)PR]-TASP constructs. Cells were then washed extensively before preparation of cell extracts. The complexes formed between the biotin-labeled TASP constructs and any cell-surface protein were then recovered by purification using avidin-agarose (“Materials and Methods”). The presence of P95 in the purified preparations was then revealed by ligand blotting using biotin-labeled 5[Kψ(CH₂N)PR]-TASP. Lanes 6 to 8: to show the specificity of complex formation between cell-surface P95 and biotin-labeled 5[Kψ(CH₂N)PR]-TASP, cells were first incubated (22°, 10 min) with excess 50 μM of unlabeled 5[KPR]-, 5[Kψ(CH₂N)PR]- and 5[QPQ]-TASP before addition of 5 μM biotin-labeled 5[Kψ(CH₂N)PR]-TASP and recovery of the complex using avidin-agarose (as above). Lane 1 “Extract” represents the ligand blot analysis of crude CEM cell extracts; material corresponding to 10⁶ cells. In all the other lanes representing the recovery of P95 from the cell surface, the material analyzed corresponded to that from 5×10⁶ cells.

* The TASP constructs which were biotinylated are referred to as TASP/B.

FIG. 7 +L. Isolation of ¹²⁵I-labeled P95 complexed to 5[Kψ(CH₂N)PR]-TASP.

Cell-surface proteins were first labeled by iodination of intact CEM cells before incubation in the absence (lanes None) or presence of 5 μM biotin-labeled 5[QPQ]- or 5[Kψ(CH₂N)PR]-TASP (lanes 2 and 3, respectively). Cells were then washed extensively before preparation of cell extracts. The complexes formed between the biotin-labeled TASP constructs and any cell-surface protein were then recovered by purification using avidin-agarose (“Materials and Methods”). The purified proteins were eluted in the electrophoresis sample buffer and analyzed by SDS/PAGE. An autoradiograph is presented (panel A). The presence of P95 in the purified preparations was confirmed by ligand blotting using biotin-labeled 5[Kψ(CH₂N)PR]-TASP (section B). The band below P95 corresponds to its hypothetical degradation product. The higher molecular weight band corresponding to a 140 kDa protein is of unknown nature; its binding was independent of TASP constructs since it was also recovered in the control avidin-agarose sample.

FIG. 8 +L. The biotin-labeled V3 loop peptide binds a 95 kDa protein on the cell surface as the biotin-labeled 5[Kψ(CH₂N)PR]-TASP inhibitor.

A. The capacity of the V3 loop peptide to bind a cell surface ligand is inhibited partially by the pseudopeptide 5[Kψ(CH₂N)PR]-TASP inhibitor. CEM cells were incubated with the V3 loop peptide (referred to as V3-biotin; at 25 mM) in the absence (peak V3-biotin) or presence of 25 μM of unlabeled 5[Kψ(CH₂N)PR]-TASP (P19+V3-biotin). FACS analysis using the biotin-labeled V3 loop peptide was as described in the “Experimental Procedures”. The peak control gives the fluorescence of cells incubated with the unlabeled 5[Kψ(CH₂N)PR]-TASP construct (25 mM). The ordinate gives the relative cell number, whereas the abscissa gives the relative fluorescence intensity.

B. The V3 loop binds and forms a stable complex with the cell surface P95. CEM cells were washed extensively with PBS before incubation (as 50×10⁶ cells per 300 μl of FACS buffer) at 22° C. for 10 min in the absence (−, lanes: 1, 3, and 5) or presence (+, lanes: 2, 4, and 6) of unlabeled 5[Kψ(CH₂N)PR]-TASP (50 μM). These suspensions were then further incubated at 4° C. for 30 min with the biotin-labeled constructs: the 5[QPQ]-TASP construct (100 μM) used as a control (lanes 1 and 2), 5[Kψ(CH₂N)PR]-TASP (10 μM), and the synthetic V3 loop peptide (100 μM) corresponding to HIV-1 isolate (lanes 5 and 6). Cells were then washed in FACS buffer and nucleus-free cell extracts were prepared and analyzed as described in the “Experimental Procedures”. The protein-complexes recovered by avidin-agarose were analyzed by SDS/PAGE, and the presence of P95 was revealed by ligand blotting using biotin-labeled 5[Kψ(CH₂N)PR]-TASP. The numbers on the left (200, 97, 68, 43, and 29) show the position of molecular weight (in kDa) protein markers. Material corresponding to 15×10⁶ cells was analyzed for each sample in lanes 1 to 6.

* The TASP constructs which were biotinylated are referred to as TASP/B*.

FIG. 9 +L. Purification of the V3 loop-BPs.

Large quantities of nucleus-free cell extracts were purified by the affinity matrix constructed using the biotin-labeled 5[Kψ(CH₂N)PR]-TASP and avidin-agarose (“Experimental Procedures”). Aliquots from the purified preparation, referred to as V3L-BPs for V3 loop binding proteins, were analyzed by PAGE/SDS using a 12.5% polyacrylamide slab gel. A part of the gel was stained with Coomassie blue to reveal the protein bands (lanes 1 and 2), and the other part of the gel was processed for ligand blotting using the biotin-labeled 5[Kψ(CH₂N)PR]-TASP (lane 3) or the biotin-labeled V3 loop peptide. The numbers on the left give the position of molecular weight (in kDa) protein markers (lane M/1). Material corresponding to 10 and 3 mg protein was analyzed in lanes 2 and 3/4, respectively.

FIG. 10 +L. Nucleolin, PHAP II and PHAP I, bind the pseudopeptide 5[Kψ(CH₂N)PR]-TASP and the V3 loop peptide.

Cytoplasmic extracts from CEM cells were used to purify proteins that bind the biotin-labeled 5[Kψ(CH₂N)PR]-TASP construct (lane 3) or the biotin-labeled V3 loop peptide (lane 4). As a control, the biotin-labeled 5[QPQ]-TASP construct was used under similar experimental conditions (lane 2). Such samples along with cytoplasmic crude extracts were analyzed by immunoblotting using the following antibodies (referred to as a): a-nucleolin peptide, a-PHAP II peptide, a-PHAP I peptide, mAb CC98 (murine monoclonal antibody specific for the human nucleolin), a-CXCR4 peptide, and a-CD4 (Neosystem). Besides mAb, all the others were rabbit antibodies. The mAb CC98 was used at 5-fold dilution of the hybridoma culture supernatant. The rabbit antisera against different proteins were used at 100-fold dilution. The numbers on the left give the position of molecular weight (in kDa) protein markers.

FIG. 11 +L. Subcellular distribution of nucleolin, PHAP II and PHAP I.

Nuclear and cytoplasmic extracts (lanes N and C, respectively) from CEM cells were prepared as described in the “Experimental Procedures”. The presence of nucleolin, PHAP II and PHAP I was revealed by immunoblotting using rabbit antisera (at 100-fold dilution) raised against synthetic peptides corresponding to the NH₂-terminus of each of these proteins. On the left is the profile of protein markers. Material corresponding to 10⁶ cells was analyzed in each lane.

FIG. 12 +L. Cell surface expressed nucleolin could be differentiated from that expressed in the nucleus.

Cell surface expressed P95/nucleolin preparation (Panel A), and crude nuclear (Panel B) and cytoplasmic (Panel C) extracts were analyzed by two dimensional gel isoelectric focusing. Experimental conditions were as described previously (Knust et al., 1982). The proteins were resolved in the second dimension on a 7.5% polyacrylamide-SDS gel. Following two dimensional gel isoelectric focusing, sections of each gel were processed by immunoblotting using rabbit polyclonal antibodies against human nucleolin. The concentration of ampholine (Pharmacia Biotech, Sweden) was at 2% of pH range 3-10. The pH gradient obtained by isoelectric focusing (first dimension) was from 4 to 7. On the left of each gel, is the profile of protein markers.

FIG. 13 +L. Expression of nucleolin, PHAP II and PHAP I in different types of human and murine cells.

A. Cell surface expression of nucleolin in human and murine cells. The cell surface expression of nucleolin was demonstrated by complex formation between 5[Kψ(CH₂N)PR]-TASP and cell surface components following incubation of intact cells with the biotin-labeled 5[Kψ(CH₂N)PR]-TASP construct (5 μM). For cells in suspension, the experimental procedures were as in the legend of FIG. 1 +L. For cells which were cultured as monolayers, cells were washed with the FACS buffer before incubation (4° C. for 30 min) with 5 μM of the biotin-labeled 5[Kψ(CH₂N)PR]-TASP. Cell monolayers were then washed with the FACS buffer and extracted as such with the buffer E (by this extraction nuclei remain attached to the culture flask). Such extracts were then centrifuged at 12,000 g (4° C., 10 min.), and the supernatants were processed as in the case of cells in suspension. Different human (HeLa, RD, Daudi, MOLT4, CEM, U937, and Jurkat) and murine (L929, T54, and T54/W12) cell lines were investigated. The samples (material corresponding from 10¹⁷ cells) were analyzed by immunoblotting using rabbit polyclonal antibodies against the purified human nucleolin. On the right is the position of P95/nucleolin and its degradation products.

B. Expression of nucleolin, PHAP II and PHAP I in different cells. Extracts from different types of human and murine cells (material corresponding to 50×10⁶ cells) were purified on the affinity column containing 5[Kψ(CH₂N)PR]-TASP in order to recover the V3 loop-BPs: nucleolin, PHAP II and PHAP I (as described in the legend of FIG. 2 +L). The purified proteins were then eluted by 2-fold electrophoresis sample buffer and analyzed by immunoblotting using rabbit antiserum (referred to as a): a-nucleolin peptide, a-PHAP II peptide, a-PHAP I peptide. The rabbit antisera against different proteins were used at 100-fold dilution. Sections corresponding to the positions of each P95/nucleolin, P40/PHAP II and P30 PHAP I are shown. The antiserum against the PHAP I peptide reacted also with a 20 kDa protein which should be a degradation product of P30/PHAP I.

As the inhibitor-TASP, the different antibodies had no significant effect on the infection of CEM cells by an HIV-pseudotyped-virus expressing Mo-MLV envelope proteins, thus pointing out their specificity to the HIV-envelope-mediated entry process. Interestingly, any one of such antibodies inhibited infection of peripheral blood mononuclear cells with the macrophage-tropic HIV-1 Ba-L and Ada-M isolate or syncytium- and non-syncytium-inducing primary HIV-1 isolates. The inventors results suggest that these Free V3-BPs serve as an anchorage point besides CD4 to allow stable and functional binding of HIV particles to permissive cells.

Interestingly, 5[Kψ(CH₂N)PR]-TASP inhibits infection of cells by HIV-1 or HIV-2 but not by SIVmac (Callebaut et al., 1996) and has no effect on HIV-1 pseudotyped with Mo-MLV (results herein) or VSV (unpublished results) envelope proteins, thus demonstrating its specific action on the HIV-envelope-mediated entry process. Here, by using an affinity matrix containing either 5[Kψ(CH₂N)PR]-TASP or a synthetic V3 loop peptide, we report the isolation of nucleolin, PHAP II and PHAP I as three V3 loop binding proteins (V3-BPs). In addition, we provide evidence for the implication of these V3-BPs in the process of HIV-particle binding to CD4⁺ cells. Although nucleolin, PHAP II and PHAP I have the ability to bind independently a synthetic V3 loop peptide or the pseudopeptide 5[Kψ(CH₂N)PR]-TASP, our results indicate that these three proteins should be functional together, since antibodies directed against any one of them inhibit the binding of HIV-particles to cells.

FIG. 14 +L. The effect of the purified V3 loop-BPs on HIV infection.

A. The HIV-1 virus inoculum was first incubated with 40, 20, and 10 mg/ml of the purified preparation of the V3 loop-BPs at 4° C. for 30 min before the addition of CEM cells (10⁶). Virus binding and entry to CEM cells was carried out by incubation at 37° C. for 1 hour. Cells were then centrifuged and suspended* in fresh culture medium (Callebaut et al., 1996). At 8 hours post-infection (p.i.), AZT (5 μM) was added to the cultures to prevent multiple cycles of infection. HIV-1 production was monitored by measuring the concentration of HIV-1 major core protein p24 in the culture supernatants at 4 days p.i. The mean of duplicate samples is shown. Heparin (histogram H) at 100 μg/ml was added 5 min before the virus, and was used as a control of inhibition of HIV-1 infection (Krust et al., 1993). The purified V3 loop-BPs preparation was as described in FIG. 2 +L.

B. The HIV-1 virus inoculum was first incubated with 20, 10, 5, 2, and 1 mg/ml of the purified preparation of the V3 loop-BPs at 4° C. for 30 min before the addition of CEM cells (10⁶). Virus binding and entry to CEM cells was carried out by incubation at 37° C. for 1 hour. Cells were then centrifuged and suspended* in fresh culture medium (Callebaut et al., 1996). HIV-1 production was monitored by measuring the concentration of HIV-1 major core protein p24 in the culture supernatants at 5 days p.i. The mean of duplicate samples is shown. The purified V3 loop-BPs preparation was as described in FIG. 2 +L.

* It should be noted that the different reagents were present only during the 1 hour incubation period with HIV-1.

FIG. 15 +L. Rabbit antisera against any one of the V3 loop-BPs inhibit HIV infection.

Rabbit antisera raised against synthetic peptides corresponding to the NH₂-terminal sequence of nucleolin, PHAP II and PHAP I, were generated as described in the “Experimental Procedures”. CEM cells were first incubated (37° C., 15 min) at different dilutions (1:200, 1:400, and 1:800) of each antiserum before infection with the HIV-1 Lai isolate, (0.2 synchronous dose). Virus production was then monitored by the concentration of p24 at 6 days p.i. The mean of duplicate samples is given. The control sample (C-Serum) corresponds to an antiserum from a rabbit immunized against the synthetic peptide 40-55 of ribonucleoprotein (RNP) U₁C.

FIG. 16 +L. Peptide-affinity purified antibodies specific to any one of the V3 loop-BPs inhibit HIV infection.

Rabbit antisera raised against synthetic peptides corresponding to the NH₂-terminal sequence of nucleolin, PHAP II and PHAP I, were purified using the corresponding synthetic peptide which was used as antigens for immunization of the rabbits (“Experimental Procedures”). CEM cells were first incubated (37° C., 15 min) with each antibody at 100 μg/ml before infection with the HIV-1 Lai isolate (0.2 synchronous dose). Virus production was then monitored by measuring the concentration of p24 at 5 and 6 days days p.i. (Sections A and B, respectively). The mean of triplicate samples is given. The control sample (C-IgG) represents rabbit antibodies against the synthetic peptide 40-55 of RNP U₁C, which was purified by protein-A sepharose.

FIG. 17 +L. Peptide purified antibodies against either nucleolin, PHAP II and PHAP I inhibit the binding of HIV particles to cells.

CEM cells (5×10⁶) were first preincubated (15 min, 37° C.) in the absence (Control) or presence of mAb anti-CD4 CB-T4 (5 μg/ml; histogram a-CD4), rabbit peptide purified antibodies against nucleolin, PHAP II and PHAP I at 100 μg/ml, or combination of antibodies as shown (at the same concentrations as when used alone) before the addition of HIV-1 Lai (material corresponding to 5 ng of p24), and further incubation at 37° C. for 1 hr. The cells were then washed and cell extracts were prepared to estimate the amount of HIV binding to cells (HIV bound on the surface+HIV entered into cells). The amount of virus particles was estimated by measuring the concentration of p24. The experimental conditions were as described previously (Krust et al., 1993; Callebaut et al., 1997b).

FIG. 18 +L. The binding of gp120 to V3 loop-BPs in 2 dose-dependent manner.

This is an ELISA-type experiment using the purified preparation of the V3 loop-BPs which was as described in FIG. 2 +L. The purified preparation of the V3 loop-BPs at different concentrations (12;5 to 200 ng/ml; abscissa) was coated to the plate before incubation with either gp120 (at 1 ng/ml; ), gp41 (at 2 ng/ml;), or histone H3 (1 μg/ml;). After extensive washing, the binding of different reagents was monitored using specific antibodies: mAb 110-D specific for residues 381-394 of gp120 of HIV-1, mAb 41-A specific for gp41, and mAb specific for histone H3 (“Experimental Procedures”). As a control mAb, we used mAb OKT4A specific for CD4. The abscissa gives the concentration of V3 loop-BPs in ng/ml. The ordinate gives the optical density (OD) values measured at 450 nm as an indicator of reactivity. An OD value less than 0.2 was considered as not significant. mAb OKT4A at 1 mg/ml gave an OD value of 0.20 when used in wells preincubated with either gp120, gp41, or histone H3. Rabbit antiserum against CXCR4 at dilutions as low as 1:200 generated an OD value of 0.15.

FIG. 19 +L. Characterization of gp120 binding to the V3 loop-BPs.

These experiments were carried out using biosensor technology as described in the “Experimental Procedures”. The purified preparation of the V3 loop-BPs was as described in FIG. 2 +L. The gp120 was that of HIV-1 Lai isolate.

A. The gp120 prevents binding of 5[Kψ(CH₂N)PR]-TASP to the V3 loop-BPs. The binding of 5[Kψ(CH₂N)PR]-TASP to the V3 loop-BPs was carried out in the presence of increasing concentrations of gp120 (the abscissa). The ordinate gives the % inhibition of 5[Kψ(CH₂N)PR]-TASP binding to the V3 loop-BPs; the 0% inhibition value represents the degree of binding in the absence of gp120. The IC₅₀ value for the inhibition of 5[Kψ(CH₂N)PR]-TASP binding to the V3 loop-BPs is around 3 nM of gp120.

B. 5[Kψ(CH₂N)PR]-TASP prevents the binding of gp120 to the V3 loop-BPs. The binding of gp120 to the V3 loop-BPs was carried out in the presence of increasing concentrations of 5[Kψ(CH₂N)PR]-TASP (the abscissa). The ordinate gives the % inhibition of gp120 binding to the V3 loop-BPs; the 0% inhibition value represents the degree of binding in the absence of 5[Kψ(CH₂N)PR]-TASP. The IC₅₀ value for the inhibition of gp120 binding to the V3 loop-BPs is around 25 nM of 5[Kψ(CH₂N)PR]-TASP.

C. The binding of gp120 to V3 loop-BPs is prevented by mAb against the V3 loop. The binding of gp120 to the V3 loop-BPs was carried out in the presence of different concentrations of mAb N11-20 against the V3 loop of the HIV-1 Lai/gp120 (the abscissa). The ordinate gives the % inhibition of gp120 binding to V3 loop-BPs; The 0% inhibition value represents the degree of binding in the absence of the antibody.

FIG. 20 +L. Recovery of nucleolin/PHAP II/PHAP I expressed on the surface of peripheral blood mononuclear cells.

Peripheral blood mononuclear cells (PBMC) from an healthy donor were activated by PHA as described previously (“Experimental Procedures”). Four days after activation, cells were washed in PBS and cytoplasmic extracts were prepared and purified on an affinity column containing the 5[Kψ(CH₂N)PR]-TASP pseudopeptide (as described in FIGS. 2 and 3 +L). In parallel, intact cells were incubated with the biotin-labeled 5[Kψ(CH₂N)PR]-TASP in order to recover complexes formed on the cell surface (all the experimental conditions were as described in the legend of FIG. 1 +L). Material purified from the cell extracts (panel Cell Extracts; material coresponding to that purified from 10⁷ cells) and from the cell surface (panel Cell Surface, material coresponding to that purified from 10⁷ cells) was analyzed by immunoblotting using rabbit antiserum against either nucleolin peptide (lanes 1 and 4), PHAP II peptide (lanes 2 and 5) and PHAP I peptide (lanes 3 and 6).

FIG. 21 +L. Synergistic inhibition of HIV infection by 5[Kψ (CH₂N)PR]-TASP peptide and AZT.

CEM cells (5×10⁶) were infected with I synchronous dose of HIV-1 Lai and the virus production was monitored in the culture supernatant 4 days p.i. by measuring the concentration of p24 (the ordinate).

A. 5[Kψ (CH₂N)PR]-TASP was added at different concentrations: 0.1 mM, 1 mM or 5 mM one hour before the virus.

B. One hour before infection AZT: 1 mM was added alone (−) or in addition to 5[Kψ (CH₂N)PR]-TASP: 0.1 mM or 1 mM.

FIG. 22 +L: Proposed shema of the stable and functional binding of HIV particles that requires P95/nucleolin, P40/PHAPII or P30/PHAPI along the CD4 molecule.

The binding of HIV particles to cells is stabilized by two distinct ineractions, both mediated by gp120/gp125, which on ne hand interacts with CD4 through the well described CD4 binding domain, and on the other hand interacts with the P95/nucleolin, P40/PHAPII or P30/PHAPI complex through thge V3 loop. As P95/nucleolin, P40/PHAPII or P30/PHAPI do not contain hydrophobic domains, then their expression on the cell surface should be dependent on the capacity of these proteins to interact wuith a still unidentifeide protein. Once the virion becomes stably attached too the cell surface, then, there would be interactions with CRXCR4 which would lead towards virus to cell membrane fusion process. Antibodies directed against the gp120/gp125 binding domain in CD4 (α-CD4), or directed against the CD4 binding domain in gp120 (α-gp120) block the binding of HIV particles to cells. Inhibition of HIV binding could also be obtained by antibodies directed against any ine of the components of the P95/nucleolin, P40/PHAPII or P30/PHAPI complex (α-nicleolin, -PHAPI, PHAPII) probably by inducing changes in the structure of the complex, by neutralizing anti-V3 loop antobodies (α-V3 loop) which block the interaction of the V3 loop with P95/nucleolin, P40/PHAPII or P30/PHAPI, by the pseudopeptide 5[Kψ (CH₂N)PR]-TASP which binds P95/nucleolin, P40/PHAPII or P30/PHAPI , and by polyanions such as heparin which by binding to the V3 loop blocks its interaction with P95/nucleolin, P40/PHAPII or P30/PHAPI. Finally, the natural ligand of CXCR4 named SDF or antibodies against CXCR4 (α-CXCR4) block HIOV infection without affecting binding of HIV particles to cells.

FIG. 23 +L. The action of rabbit antisera raised against nucleolin, PHAP II and PHAP I peptides is specific to the HIV envelope glycoproteins.

CEM cells (2×10⁵) were infected with the HIV-1 pseudoryped Mo-MLV virus (Experimental procedure) at 95 ng of p24/ml, in the absence (histogram Control) or presence of different additions (as indicated). The infection was monitored by the production of p24, which was measured in the culture supernatant at 48 hours p.i. The mean±SD of duplicate samples is given. Treatment of cells with the different antibodies was as in FIG. 6 +L. For a control of infection, cells were pretreated with 5 μM AZT before infection. Treatment with 5[Kψ(CH₂N)PR]-TASP was at 5 μM.

FIG. 24 +L. Peptide affinity purified antibodies specific to nucleolin, PHAP II and PHAP I inhibit HIV infection.

CEM cells were first incubated (37° C., 15 min) with the different antibodies (the symbols a) or reagents before infection with the HIV-1 Lai isolate (0.2 synchronous dose; Cellebaut et al., 1996). Virus production was then monitored by measuring the concentration of p24 at 5 days p.i. The results give HIV production as a percentage of the control sample (135≅19 ng/ml p24) infected without any addition. The mean±SD of triplicate samples is given. Rabbit polyclonal antibodies against nucleolin, PHAP II, and PHAP I were purified by affinity chromatography using their respective peptide antigen. MAbs anti-CD4 (CB-T4; Velenzuela et al., 1997) and anti-CD45 (Hook et al., 1991), and rabbit polyclonal antibodies against adenosine deaminase (ADA; Martin et al., 1995), were purified by protein-G sepharose. The histogram C-Ab represents immunoglobulins from a rabbit injected five times with adjuvant alone and was purified by protein-A sepharose. Rabbit antibodies were used at 100 μg/ml, whereas the mAbs were used at 5 μg/ml. SDF1α and 5[Kψ(CH₂N)PR]-TASP were used at 0.2 and 5 μM, respectively. The different antibodies and reagents were added only at the time of infection and at 3 days p.i.

FIG. 25 +L. Peptide affinity purified antibodies specific to nucleolin, PHAP II and PHAP I inhibit infection of PBMC by different HIV-1 isolates.

A. The effect on macrophage-tropic HIV-1 Ba-L and Ada isolates. The PHA-activated PBMC (Callebaut et al., 1996) were infected by the HIV-1 isolates at a dose corresponding to 25 ng/ml of p24. The virus production in cultures infected by HIV-1 Ba-L and Ada was monitored at 9 and 11 days p.i., respectively. B/C. The effect on a syncytium-inducing (92UG029A) and a non-syncytium-inducing (92BR025C) primary HIV-1 isolate. The non-syncytium-inducing isolate manifested a slow/low growing phenotype compared to the syncytium-inducing isolate (Callebaut et al., 1996), therefore, virus production was monitored at 13 and 7 days p.i., respectively.

In sections A/B/C, the mean±SD of duplicate samples is given. The histogram Control represents the production of virus in cultures without the addition of antibodies. The histogram C-Ab represents a purified preparation of antibodies from a rabbit injected five times with adjuvant alone and was purified by protein-A sepharose. The histogram C-Ab* represents a purified preparation of antibodies from a non-immunized rabbit. The affinity purified antibodies against nucleolin, PHAP II and PHAP I were as in FIGS. 7-9 +L. The different antibodies (at 100 μg/ml) were added only at the time of infection and at 3 days p.i.

FIG. 26 +L. Nucleolin, PHAP II and PHAP I, bind the pseudopeptide 5[Kψ(CH₂N)PR]-TASP and the V3 loop peptide.

Aliquots of avidin-agarose (30 ml) in PBS-EDTA were incubated (18 hr, 4° C.) in the presence of either the biotin-labeled control 5[QPQ]-TASP construct (100 μM; lanes 2), the biotin-labeled 5[Kψ(CH₂N)PR]-TASP (20 μM; lanes 3), or the biotin-labeled V3 loop peptide (100 μM; lanes 4) before washing extensively in PBS-EDTA. Cell extracts (materials corresponding to 15×10⁶ cells) were then added to the affinity matrix and after 2 hr of incubation at 4° C., the samples were washed extensively with PBS-EDTA. The purified proteins were eluted by the addition of 2-fold concentrated electrophoresis sample buffer and analyzed by SDS/PAGE (Callebaut et al., 1997). Such samples (lanes 2-4) along with cytoplasmic crude extracts (Lanes 1) were analyzed by immunoblotting using the following rabbit antisera (referred to as a): A, α-nucleolin; B, α-PHAP II; C, α-PHAP I; E, α-CXCR4; F, α-CD4 (Neosystem); and finally D, mAb CC98 (murine monoclonal antibody specific for the human nucleolin). The mAb CC98 was used at 5-fold dilution of the hybridoma culture supernatant. The rabbit antisera against different proteins were used at 100-fold dilution. The antibodies were revealed with specific immunoglobulins labeled with horseradish peroxidase (Amersham). The numbers on the left give the position of molecular weight (in kDa) protein markers.

FIG. 27 +L. Affinity purified antibodies directed against either nucleolin, PHAP II or PHAP I peptides inhibit the binding of HIV particles to cells.

CEM cells (5×10⁶) were first preincubated (15 min, 37° C.) in the presence of the different antibodies (concentrations as in FIG. 6 +L) or reagents separately or combination of antibodies as shown (at the same concentrations as when used alone) before the addition of HIV-1 Lai (material corresponding to 50 ng of p24), and further incubation at 37° C. for 1 hr. The cells were then washed and cell extracts were prepared to estimate the amount of HIV binding to cells (HIV bound on the surface+HIV entered into cells). SDF1α and 5[Kψ(CH₂N)PR]-TASP were used at 0.5 and 5 μM, respectively. The amount of virus particles was estimated by measuring the concentration of p24. The results give the percentage of HIV bound in respect to that observed for the control sample (282±44 ng/ml p24) incubated without any addition. The mean±SD of duplicate samples is given. The experimental conditions were as described previously (Krust et al., 1993; Valenzuela et al., 1997).

FIG. 28 +L: The inhibition of HIV entry into HeLa cells by 5[Kψ (CH₂N)PR]-TASP is specific to the HIV envelope glycoproteins.

FIG. 29 +L: 5[Kψ (CH₂N)PR]-TASP inhibits entry of HIV-1 isolates resistant to antiviral drugs.

FIG. 30 +L: 5[Kψ (CH₂N)PR]-TASP inhibits entry of different HIV-1 isolates in Peripheral Blood Monoculear Cells (PBMC).

FIG. 31 +L: Chemokines inhibit poorly HIV infection in HeLa cells

FIG. 32 +L: Association of chemokines and 5[Kψ (CH₂N)PR]-TASP results in a synergistic effect on HIV infection in PBMC.

FIG. 33 +L: 5[Kψ (CH₂N)PR]-TASP inhibits HIV entry by its capacity to bind the surface of HeLa cells.

FIG. 34 +L: 5[Kψ (CH₂N)PR]-TASP inhibits the binding and entry of HIV particles.

FIG. 35 +L: 5[Kψ (CH₂N)PR]-TASP binds and becomes complexed with the cell surface expressed nucleolin (P95).

FIG. 36 +L: The binding of 5[Kψ (CH₂N)PR]-TASP to the cell surface expressed nucleolin (P95) results in its cleavage.

Lane 1: 5[Kψ (CH₂N)PR]-TASP0 μM 1 h incubation time;

Lane 2: 5[Kψ (CH₂N)PR]-TASP5 μM 1 h incubation time;

Lane 3: 5[Kψ (CH₂N)PR]-TASP5 μM 6 h incubation time;

Lane 4: 5[Kψ (CH₂N)PR]-TASP5 μM 24 h incubation time;

FIG. 37 +L: The anti-HIV effect of heparin is not correlated with the anti-HIV effect of 5[Kψ (CH₂N)PR]-TASP.

FIG. 38 +L: The distribution of PHAP II in CEM cells following the use of rabbit polyclonal anti-P40 (Shangai) antibody.

a) Gold particles are numerous within the closed intranuclear vesicles (arrow).

b) Four gold particles are associated with the exocytose vesicles (arrow).

c) A few gold particles are present along the plasma membrane (arrows). Bars=0.5 μm.

FIG. 39 +L: The distribution of PHAP I in CEM cells following the use of rabbit polygonal anti-P30 antibody.

a) Gold particles are numerous over the cytoplasm [C] and the nucleus (N). In the cytoplasm, a closed vesicle (arrow) and an exocytose vesicle (double arrow) contain gold particles. In addition, a few gold particles are associated with the plasma membrane (arrowhead).

b) As above, gold particles are present in the cytoplasm [C] and the nucleus (N). The arrow points to an intracytoplasmic closed vesicles located near the cell surface.

Bars=0.5 μm.

FIG. 40 +L: The distribution of nucleolin (P95) in CEM cells following the use of rabbit polyclonal antibodies raised against purified human nucleolin.

a) The arrow points to a labeled closed vesicle which is adjacent to the plasma membrane. C: Cytoplasm.

b) Gold particles are present at the cell surface (arrowheads) and in a clear closed vesicle adjacent to the plasma membrane (arrow). C: Cytoplasm.

c) Gold particles are randomly scattered over the cytoplasm (C) and this part of nucleus (N). In addition, they are present in an exocytose vesicle (arrow).

Bars: 0.5 μm.

FIG. 41 +L: Membrane labeling of monocyte-derived macrophages (MDM). After a 7 days culture time period, cells are labeled with the following monoclonal antibodies:

a) CD45Ro, CD11b and CD14;

b) CD64, CBT4 (CD4), 2D7 (CCR5), 12G5 (CXCR4) and 7B12 (CCR3).

Auto=Auto-fluorescence.

5×10⁵ cells are incubated in the presence of the respective above-cited monoclonal antibody (10 μg/ml) during 30 min at 4° C. After three washings with a PBS/BSA/Azide solution, cells are incubated during 30 min with an FITC-labeled anti-IgG antibody (dilution: 1/500). Cells are fixed in 500 μl of PBS/performaldehyde (PFA) 1% before FACScan analysis.

FIG. 42 +L: Binding specificity of 5[Kψ (CH₂N)PR]-TASP and of anti-V3BPs antibodies on the macrohage surface:

a) binding of 5[Kψ (CH₂N)PR]-TASP-FITC (P19*) on the macrophage surface and binding competition with 5[Kψ (CH₂N)PR]-TASP (P19). C: Control.

b) binding of the anti-V3BPs antibodies on the macrophage surface. Auto=Auto-fluorescence.

5×10⁵ cells are labeled directly with 2 μM of 5[Kψ (CH₂N)PR]-TASP-FITC (P19*) or indirectly with 10 μg/ml antibodies, during 30 min at 4° C. After three washings, in a PBS/BSA/Azid solution, cells are fixed with PBS/performaldehyde (PFA) 1% (500 μl). For the indirect labeling, cells are incubated in the presence of 100 μl of a rabbit anti-IgG antibody labeled with FITC (dilution: 1/500) during 30 min before washing the cell culture and fixing the cells.

FIG. 43 +L: Purification of V3BPs from macrophages.

Cell extracts obtained from eight days culture (D8) macrophages are incubated with 20 μM of biotinylated 5[Kψ (CH₂N)PR]-TASP that has been previously bound to avidin-agarose, before the purified V3BPs are revealed by Western blot analysis.

The presence of P95, P40 and P30 has been revealed in the Western blot experiment using a mixture of three antibodies, each of these antibodies being directed against each of P95, P40 and P30. The 60 kDa and 80 kDa Mw protein bands correspond to the degraded forms of P95. Cell extracts from CEM cells are used as a positive control.

FIG. 44 +L: Inhibition of the macrophage infection with HIV-1 by 5[Kψ (CH₂N)PR]-TASP.

Macrophages are infected with BaL or Ada HIV-1 viruses, in the presence of 2 μM, 1 μM or 0.1 μM of 5[Kψ (CH₂N)PR]-TASP. Cells are preincubated during 30 min at 4° C. in the presence of 5[Kψ (CH₂N)PR]-TASP before adding the virus. Every 3 days interval, the whole culture supernatant is collected and is replaced by RPMI 1640 culture medium supplemented with 20% fetal calf serum (FCS), 1% antibiotics, 1% glutamine, in addition to 5[Kψ (CH₂N)PR]-TASP that is kept present in the culture medium until day 14 after in vitro infection. Virus production is quantitated by titration of p24 protein in the culture supernatant at different times (days) after in vitro infection. Each value represented in the graphs represents the mean of two test samples. These results are representative of three independent experiments.

FIG. 45 +L: Inhibition of the macrophage infection with HIV-1 by anti-P95, anti-P40 and anti-P30 antibodies.

Macrophages are infected by the BaL VIH-1 virus in the presence of 100 μg/ml of anti-P95, anti-P40 and anti-P30 antibody. Cells are preincubated during 30 min at 4° C. in the presence of each kind of antibody before adding the virus. The antibodies are kept present in the culture medium during the whole infection period of time. The experimental conditions are identical to those detailed in 4 +L4. The results are representative of two independent experiments.

FIG. 46 +L: Inhibition of the macrophage infection with HIV-1 by β chemokines and by anti-V3BPs antibodies.

Macrophages are infected with BaL VIH-1 virus in the presence of [(Rantes/anti-P40 or anti-P30), (MIP-1α/anti-P40 or anti-P30) or also (MIP-1β/anti-P40 or anti-P30)].

Cells are preincubated during 30 min at 4° C. in the presence of the different molecules either alone or in association before adding the virus. Every three days period, the whole culture supernatant is collected and is replaced by RPMI 1640 culture medium supplemented with 20% fetal calf serum (FCS), 1% antibiotics, 1% glutamine, in addition to each tested molecule. Virus production is quantitated by titration of p24 protein in the culture supernatant at different times (days) after in vitro infection. Every value reprented in the graphs represents the mean of two test samples.

FIG. 47 +L: Inhibition of the macrophage infection with HIV-1 by β chemokines and 5[Kψ (CH₂N)PR]-TASP. Macrophages are infected with BaL HIV-1 virus in the presence of [Rantes/5 [Kψ (CH₂N)PR]-TASP), (MIP-1α/5[Kψ (CH₂N)PR]-TASP) or also (MIP-1β/5[Kψ (CH₂N)PR]-TASP)]. The indicated percentage values represent the different inhibition values with, on one hand, β chemokines or 5[Kψ (CH₂N)PR]-TASP alone, and on the other hand with an association of these molecules. The experimental conditions are identical to those described in the legend of FIG. 46 +L.

FIG. 48 +L: 5[Kψ (CH₂N)PR]-TASP and the V3 loop peptide do not bind PHAP-I deleted in its acidic region.

Wild type (aa 1-249) and deleted (aa 1-167) PHAP I both fused with the Histidine Tag His₆ were produced in the yeast system Pichia pastoris expression system (Invitrogen), and purified with Ni²⁺ charged columns according to manufacturor's instructions (Ni—NTA, QIAGEN). Aliquots of the purified proteins, wild type (lanes 1, 3, 5) and deleted (lanes 2, 4, 6), were analyzed by SDS/PAGE. Such samples were analyzed by immunoblotting using the rabbit polyclonal antibodies raised against the synthetic N-terminal peptide corresponding to the PHAP I sequence (1/500^{e} dilution of serum; lanes 1, 2), or by ligand-blotting (Callebaut et al., 1997) in the presence of either the biotin-labeled 5[Kψ (CH₂N)PR]-TASP (5 μM; lanes 3, 4) or the biotin-labeled V3 loop peptide (25 μM; lanes 5, 6). The antibodies and the biotin-labeled molecules were revealed with specific immunoglobulins labeled with horseradish peroxidase (-HLP) and streptavidin-HRP respectively (amersham). The numbers on the left give the position of molecular weight (in kDa) protein markers. Material corresponding to 1 μg protein was analyzed in each lane.

We have previously suggested that the capacity of nucleolin, PHAP II, and PHAP I to bind 5[Kψ (CH₂N)PR]-TASP and gp120 is due to the presence of acidic domains (amino acids glutamate and aspartate) in these V3 loop binding proteins. Here we demonstrate that recombinant PHAP I binds 5[Kψ (CH₂N)PR]-TASP or the V3 loop, however PHAP I devoid of its C-terminal acidic domain does not bind. These results therefore illustrate that the acidic domain could indeed account for the capacity of the V3 loop binding proteins to bind 5[Kψ (CH₂N)PR]-TASP or the V3 loop.

Figure 49
I. Aminoacid and II genomic DNA sequences and mRNA of the P95/nucleolin protein.

| | |
|---|---|
| exons | : 1070. .1198, 2159. .2275, 3439. .3916,4587. .4784,4889. .4975,5160. |
| | .5301,6307. .6431,7037. .7160,7620. .7777,8292. .8415,4852. .8785,9279. |
| | .9405,9792. .10006,10140.10499 |
| mRNA | join(1070. .1198,2159. .2275,3439. .3916,4587. .4784,4889. .4975,5160. |
| | .5301,6307. .6431,7037. .7160,7620. .7777,8292. .8415,8652. .8785,9279. |
| | .9405,9792. .10006,10140. .10499) |
| CDS | join(1181. .1198,2159. .2275,3439. .3916,4587. .4784,4889. .4975,5160. |
| | .5301,6307. .6431,7037. .7160,7620. .7777,8292. .8415,8652. .8785,9279. .9405,9792. |
| | .10006,10140. .10216) |

Materials and Methods

I. Materials

Recombinant gp120 and gp41 corresponded to the external and transmembrane envelope glycoprotein, respectively, of HIV-1 Lai (IIIB), purchased from Neosystem Laboratories, Strasbourg. Recombinant gp120 is produced by the baculovirus expression system, whereas recombinant gp41 was produced by the E. coli expression system. Recombinant soluble CD4 was produced in baculovirus expression sysem and was purchased from Neosystem. Other recombinant preparations of gp120 corresponding to that of HIV-1 isolates, MN, SF2 (from Dr. K. Steimer, Chiron Corporation), LA V(or Lai), and the nonglycosylated gp120 of HIV-1 SF2 (Env 2-3; from Dr. K. Steimer; Chiron Corporation) were obtained through the AIDS Research and Reference Reagent Program, Division of AIDS, NIAID, NIH. The gp120 MN and LA V are produced in insect cells using the baculovirus expression system, gp120 SF2 is produced in CHO cells, whereas the nonglycosylated gp120 SF2 is produced in the yeast.

A. Antibodies

The monoclonal antibody (mAb) CC98 against human nucleolin (Chen et al., 1991; Fang and Yeh, 1993) was generously provided by Dr. N.-H. Yeh, Graduate School of Microbiology and Immunology, National Yang-Ming Medical College, Shih-Pai, Taiwan, Republic of China. Rabbit antiserum raised against a purified preparaion of human nucleolin was generously provided by Drs. M. Erard and C. Faucher, Centre de Recherche de Biochimie et de Génétique Cellulaire du CNRS, Toulouse, France. The mAb specific to human CD4 and reacting with the gp120 binding domain was kindly provided by Dr. E. Bosmans (Eurogenetics, Tessenderlo, Belgium). Another mAb specific to human CD4 and reacting with the gp120 binding domain, mAb OKT4A, was purchased from Ortho Diagnostics Systems. The mAb specific for histone H3 was produced in the laboratory (Benkirane et al., 1996). MAb N11/20 against the V3 loop of gp120, mAb 110/C against an epitope in gp120 corresponding to fragment 282-284 amino acids, mAb 110/D against an epitope situated at residues 381-394, mAb 41-A against gp41 (both gp120 and gp41 of HIV-1), and mAb 125-A against the extenal envelope glycoprotein of HIV-2 were provided by Dr. J. C. Mazie, Hybridolab, Institut Pasteur. MAb 110-4 against the V3 loop and mAb 110-1 against the C-terminal domain of gp120 (Kinney-Thomas et al., 1988; Linsley et al., 1988) were obtained from Genetics Systems (Seattle, Wash.). MAb ADP390 against the CD4 binding domain in gp120 (from Drs. J. Cordell and C. Dean) was provided by MRC AIDS Directed Programme Reagent Repository (McKeating et al., 1992). MA b AD3 against the first 204 amino acids of gp120 (From Drs. K. Ugen and D. Weiner), mAb V3-21 against the INCTRPN sequence at residues 298-304 containing the N-terminal end of the V3 loop (from Dr. J. Laman), and MAb b12 against the CD4 binding domain in gp120 (from Drs. D. Burton and C. Barbas), were obtained through the AIDS Research and Reference Reagent Program, Division of AIDS, NIAID, NIH (Ugen et al., 1993; Laman et al., 1992; Burton et al., 1991). Besides mAb b12 which is a human monoclonal antibody, all the other mAbs were of murine origin.

B. Cells

CEM cells (clone 13) derived from human lymphoid cell line CEM (ATCC-CCL 119), MOLT4-T4 clone 8 cells selected for high level of CD4 expression (both cell lines were provided by L. Montagnier, Institut Pasteur, France), Daudi (a Burkitt's lymphoma cell line) and U937 (a promonocytic leukemia cell line) were cultured in the suspension medium RPMI-1640 (Bio-Whittaker, Verviers, Belgium). Human HeLa (human cervix carcinoma) and RD (human rhabdomyosarcoma) cells, and murine L929 cells (fibroblast-like cells derived from normal subcutaneous areolar and adipose tissue from C3H mouse) were grown as monolayers in Dulbecco's medium. Murine hybridoma T-cell lines, T54 and T54/W12 expressing human CD4 and human CD4/CD26 were cultured in the suspension medium RPMI-1640 as described (Blanco et al., 1996). Human peripheral blood mononuclear cells (PBMC) from an healthy donor were stimulated by phytohemagglutinin (PHA) or protein A and cultured in RPMI 1640 medium containing 10% (v/v) T cell growth factor (Biotest) (Callebaut et al., 1996). All cells were cultured with 10% (v/v) heat inactivated (56° C., 30 min) fetal calf serum.

II. Methods

A. Cell Surface Iodination of Cells

CEM cells (10⁸ cells) were washed with PBS (2×25 ml) and the pellet was suspended in 20 ml of PBS containing 10 mM D-glucose and 2 mCi of ¹²⁵I (100 mCi/ml; Amersham), 2 U of lactoperoxidase, and 2 U of glucose oxydase (Calbiochem-Behring). After 10 min of incubation at 22° C., cells were washed in PBS and extracts were prepared as above.

B. FITC Labeling of TASP Constructs

5[Kψ(CH₂N)PR]-TASP was labeled with fluorescein isothiocyanate (FITC; Sigma) by incubating stoichiometric concentrations (2.5 mM) of each product in 50 mM NaHCO₃, pH 9.5, at 20° C. for 2 hr (in the dark). This solution (400 μl) was then transferred to a Microcon Model 3 filter sieve (Amicon, Inc. MA, USA) with a molecular weight cut-off of 3,000 Daltons and centrifuged at 12,000 g for 30 min to filter unbound FITC. The concentrated material was diluted 20 fold in distilled water and purified again using the Microcon filter.

C. Detection of Cell-surface Antigens

Phycoerythrin (PE)-labeled mAb Ta1 (IgG1; from Coulter, Miami, USA) was used to detect CD26 (Blanco et al., 1996). Two different FITC-labeled mAbs specific to the CD4 receptor were used, mAbs OKT4 and OKT4A (both IgG1; from Ortho Diagnostics Systems, Raritan, N.J.). In all experiments, PE-labeled mAb B4 (IgG1) specific for CD19 (Coulter) was used as a control for PE-labeled mAb Ta1, and FITC-labeled mouse isotype control antibody MCG1 (IgG1; from Immuno Quality Products) was used as a control for FITC-labeled mAbs OKT4 and OKT4A. Cells were incubated with FITC- or PE-labeled mAbs in the fluorescence activated cell sorting (FACS) buffer at 4° C. for 30 min. The cells were then washed twice with PBS and fixed in 1% formaldehyde in PBS and applied to a FACS scan flow cytometer (Beckton Dickinson, Mountain View, Calif. USA). For each sample, 10,000 cells were analysed with Lysis II Software (Beckton Dickinson).

In order to assay for the binding of FITC- or biotin-labeled TASP inhibitors to a cell-surface antigen, different cells were washed in PBS, suspended in FACS buffer (as 5×10⁵ cells per 100 μl) containing 0.5 μM FITC-labeled or different concentrations of the biotin-labeled TASP constructs, and incubated at 4° C. for 30 min. The cells were then washed twice with FACS buffer and fixed in 1% formaldehyde in FACS buffer. The FITC-labeled TASP constructs were analyzed as above, whereas the biotin-labeled TASP constructs were revealed by using streptavidin-FITC complex (Amersham). The fluorescence intensity was monitored by FACS analysis using Lysis II Software in FIGS. 3/4 and Tables 1/2, or Cell Quest™ Software (Beckton Dickinson and Apple Computer) in FIG. 5 +L.

D. Protease Treatment of Cells

Protease treatment of CEM and MOLT cells was essentially as described previously (Borrow et al., 1992) with slight modifications. Briefly, cells were washed in PBS and in RPMI-1640 medium containing 1 mM EDTA before treatment with trypsin (Sigma; 2.5 mg/ml at 20° C. for 5 min), proteinase K (Bohringer Mannhein GmbH, Germany; 0.2 mg/ml at 37° C. for 30 min), or pronase E (Sigma, 0.1 mg/ml at 37° C. for 45 min). The reactions were stopped by 10 fold dilutions in RPMI-1640 containing 10% fetal calf serum. Cells were then washed in PBS and in FACS buffer, and processed for FACS analysis.

E. Gel Filtration Chromatography

A Superose 6 column (1.6×50 cm) from Pharmacia was equilibrated in buffer GF as described before (Jacotot et al., 1996). The bed volume was 100 ml. The column was calibrated using extracts (prepared in Buffer E) supplemented with molecular mass markers: catalase, 202 kDa and bovine serum albumin, 68 kDa. Elution was in buffer GF by collecting 1 ml fractions/2 min; with the void volume (Vo) and total column elution volume (Vc) at 36 and 114 ml, respectively. Aliquots from each fraction were analyzed by ligand blotting using biotin-labeled 5[Kψ(CH₂N)PR]-TASP. Aliquots were also assayed for dipeptidyl peptidase IV (DPP IV) activity of CD26 and DPP IV-b by the cleavage of Gly-Pro-para-nitroanilide as described previously (Jacotot et al., 1996). Under these experimental conditions, CD26 and DPP IV-b eluted as monomers of 110 and 82 kDa, respectively, and these were used as convenient markers to monitor the elution profile of the TASP ligand P95.

F. Plasma Membrane Preparation

CEM cells (300×10⁶) were washed in PBS before homogenization to prepare plasma membranes, as described before (Jacotot et al., 1996). The presence of TASP ligand P95 was revealed by ligand blotting using aliquots corresponding to material from 10⁸ cells.

G. Preparation of ¹²⁵I-labeled gp120

Recombinant gp120 (Neosystem, Strasbourg, France) was radioiodinated with the Bolton-Hunter reagent (New England Nuclear-Du Pont, Boston, Mass.) according to the technique described by the manufacturer. To study the binding of gp120 to the CD4 receptor, CEM cells (5×10⁶) which express high levels of CD4 were incubated in the culture medium with ¹²⁵I-labeled gp120 (50 ng; 10 Ci/mg) at 37° C. for 1 hour. Cells were then washed twice in PBS (5 ml) and cytoplasmic extracts were prepared by disruption of cell pellets in buffer E (125 ml). Aliquots (25 ml; corresponding to material from 10⁶ cells) were diluted in two-fold concentrated electrophoresis buffer and were analyzed by SDS/PAGE. The binding of ¹²⁵I-labeled gp120 to the CD4 receptor was then revealed by autoradiography (Krust et al., 1993). The ¹²⁵I-labeled gp120 band was also quantitated in a Phosphorimager (Molecular Dynamics, Sunnyvale, Calif.).

H. Binding of HIV Particles to CEM Cells

CEM cells (5×10⁶) in culture medium (1 ml) were preincubated (at 37° C. for 15 min) in the absence or presence of 5[Kψ(CH₂N)PR]-TASP or mAb against CD4 before addition of HIV-1 Lai (corresponding to 25 ng of p24). After incubation at 37° C. for 1 hour with gentle shaking, cells were diluted 10 fold in the culture medium and pelleted by centrifugation. Cells at 4° C. were washed once in RPMI-1640 medium (5 ml) containing 1 mM EDTA, and then washed twice in RPMI-1640 medum (2×5 ml). Cell extracts were prepared in buffer E (50 ml), the nuclei were pelleted by centrifugation, and the supernatant was asayed for the concentration of p24. It should be noted that under these experimental conditions, the values for the bound virus represent particles bound on the cell surface as well as particles (or cores) entered into cells.

I. HIV Infection

Infection of CEM cells with the HIV-1 Lai isolate was carried out as described previously (Callebaut et al., 1996). For the assay of the inhibitory effect of rabbit antisera or purified antibodies, CEM cells were first incubated (15-30 min, at room temperature) in the presence of different concentrations of each antibody preparation before infection using 0.2 synchronous dose of HIV-1 Lai as described before (Laurent-Crawford and Hovanessian, 1993). HIV production was estimated at different days post-infection (p.i.) by monitoring the HIV-1 major core protein p24 in the culture supernatant (Callebaut et al., 1996). The concentration of p24 was measured by p24 Core Profile ELISA (Du Pont). For a single cycle of HIV, AZT (5 mM) was added at 8 hr post-infection to inhibit multiple cycles of virus infection; HIV production was monitored at 4 days post-infection (Laurent-Crawford and Hovanessian, 1993).

J. Buffers

Buffer E contains 20 mM Tris HCl, pH 7.6, 150 mM NaCl, 5 mM MgCl₂, 0.2 mM PMSF, 5 mM b-mercaptoethanol, aprotinin (1000 U/ml) and 0.5% Triton X-100. Buffer I contains 20 mM Tris HCl, pH 7.6, 50 mM KCl, 400 mM NaCl, 1 mM EDTA, 0.2 mM PMSF, 5 mM b-mercaptoethanol, aprotinin (1000 U/ml), 1% Triton X-100 and 20% glycerol (v/v). Buffer BIM contains 10 mM Tris HCl, pH 7.6, 25 mM KCl, 100 mM NaCl, 1 mM EDTA, 0.2 mM PMSF, 5 mM b-mercaptoethanol, 1% Triton X-100 and 20% glycerol (v/v). Tris-buffered-saline buffer contains 25 mM Tris HCl, pH 7.0, 137 mM NaCl and 3 mM KCl. Fluorescence-activated cell sorting (FACS) buffer contains 1% bovine serum albumin and 0.02% sodium azide in phosphate buffered saline (PBS). NaCl (1 M) elution buffer contains 20 mM Tris HCl, pH 7.6, 50 mM KCl, 1 M NaCl, 1 mM EDTA, 1 mM PMSF, 5 mM b-mercaptoethanol, and 20% glycerol (v/v). Dialysis buffer contains PBS, 0.1 mM EDTA, 1 mM PMSF. Two-fold concentrated electrophoresis sample buffer contains 125 mM Tris-HCl, pH 6.8, 2 M urea, 1% SDS, 0.1% bromophenol blue, 150 mM b-mercaptoethanol, and 20% glycerol, (v/v).

K. Preparation of Cell Extracts

In all the different experiments, cells were analyzed 24 hours after cell passaging. In routine experiments, for the preparation of cytoplasmic extracts, cells were first washed extensively in PBS before lysis in buffer E (150 μl per 5×10⁷ cells) and the nuclei were pelleted by centrifugation (1,000 g for 5 min). The nuclei-free supernatant was then further centrifuged at 12,000 g for 10 min, and the supernatant was stored at −80° C.

Preparation of cytoplasmic and nuclear extracts was carried out as follows. CEM cells were lysed in buffer E (900 μl per 3×10⁸ cells) and were centrifuged as above for the preparation of the cytoplasmic extracts. For the preparation of nuclear extracts, the nuclear pellet was first washed in buffer E (900 μl) and the nuclear pellet was disrupted in buffer I (600 μl), sonicated in an ice-water beaker before the addition of buffer BIM (300 μl). This suspension was left for 30 min at 4° C. before centrifugation at 12,000 g for 10 min. The supernatant containing the nuclear proteins was stored at −80° C.

L. Synthesis of TASP Constructs

Synthesis of the different TASP constructs and the measurement of their inhibitory activity on HIV infection were as described previously (Callebaut et al., 1996). The following TASP constructs were included in this report: A) control peptides which manifest no or little activity against HIV infection, such as 5[QPQ]-, and 5[KER]-TASP; B) peptides which are potent inhibitors of HIV entry and infection, such as 5[KPR]- and 5[Kψ(CH₂N)PR]-TASP. For the preparation of the biotin-labeled constructs, biotin was incorporated at the beginning of the synthesis by coupling the Fmoc-Lys(Biotin)-OH derivative (Neosystem, Strasbourg, France) an the resin prior to the assembly of the template (Callebaut et al., 1997b). Thus the biotinylated TASP constructs were labeled at the COOH-terminus of their templates.

M. Synthesis of the Biotin-labeled Cyclic V3 Loop Peptide

The V3 loop sequence corresponded to that from the HIV-1 Lai isolate (Myers et al., 1994). It contained 40 amino acids NCTRPNNNRKSIRIQRGPGRAFVTIGKIGNMRQAHCNIS. The other V3 loop sequence corresponded to that from the HIV-1 Ba-L which 39 aminoacids sequence is: NCTRPNNNTRKSIHIGPGRAFYTGEIIGDIRQAHCNLS. The biotin-labeled V3 loop peptide was synthesized using classical Fmoc chemistry. Assembly of the protected peptide chain was carried out on a 25 mmol scale; the starting Fmoc Ser (tBu) wang resin is commercially available. The protecting groups for the side chains were tBu (Ser, Thr), Trt (Asn, Glu, His, Cys), Pmc (Arg), Boc (Lys). Assembly of the amino acids was realized according to a procedure described previously, using a multichannel peptide synthesizer (Neimark and Briand, 1993). Biotin was coupled to the peptide according to the procedure used to couple amino acids (thus the biotin was at the NH₂-terminal of the peptide) and after the last step of deprotection. The biotinyl-V3 loop peptide-resin was then washed 3 times with dichloromethane, dried using ether, deprotected and cleaved from the resin using 6 ml of King's reagent (King et al., 1990). The total cleavage time was 2 h 30 min. The cleaved peptide was filtered before precipitation using cold (0° C.) ether. After centrifugation, the pellet was washed twice (10 min each time) with ether. After the last centrifugation, the pellet was solubilized in 15 ml of 10% acetic acid (v/v), and then in 1000 ml of water. The pH of the solution was raised to 9 using 1N NaOH. Taking advantage of the cysteine residues at the NH₂- and COOH-termini of the peptide, the loop structure was generated by air oxidation for 3 days under vigorous stirring. Finally, the pH was adjusted to 4 and the cyclized peptide was concentrated on a C₁₈ column eluted with 60% acetonitrile in water and 0.1% trifluoroacetic acid. After lyophilization, the crude cyclised peptide (100 mg) was purified by a semi-preparative HPLC system (ABI Perkin Elmer) using a prep-10 Brownlee column (1×10 cm; particle size of 20 mm) and a gradient of acetonitrile 0% to 80% in 0.1% trifluoroacetic acid. The final product (15 mg) was 91% pure with a mass M+H⁺ of 4706.72; the expected mass being 4707.

N. Ligand Blotting

Crude cell extracts or purified preparations of V3 loop-BPs were diluted in 2-fold concentrated electrophoresis sample buffer and analyzed by polyacrylamide gel electrophoresis in SDS (SDS/PAGE) to be electrophoretically transferred to 0.22 mm PVDF sheets (BIO-RAD). The electrophoretic blots were saturated with the casein-based blocking buffer (GENOSYS) overnight at 4° C. In order to further saturate nonspecific binding sites, the blots were first incubated at room temperature in blocking buffer containing 5 μM of 5[KER]-TASP. After 2 hr, the biotin-labeled 5[Kψ(CH₂N)PR]-TASP (5 μM) was added in this solution, and the blots were incubated for another 2 hr at 4° C. The sheets were subsequently washed 3 times (each 10 min) in Tris-buffered saline containing 0.05% (v/v) Tween 20, followed by 2 washes (each 10 min) in Tris-buffered-saline before revealing biotin by using streptavidin-horseradish-peroxidase complex and light based enhanced chemiluminescence reagents as provided by the manufacturer (Amersham). The enhanced light signal produced was then captured on the autoradiography film (Hyperfilm™-MP from Amersham). Ligand blotting with the biotin-labeled V3 loop peptide was carried out under similar conditions as above, but the concentration of the V3 loop peptide was 25 μM.

O. Immunoblot Assay

Samples in the electrophoresis sample buffer were analyzed by SDS-polyacrylamide gel (10%) electrophoresis before processing for immunoblot assay (Jacotot et al., 1996b) using mAb CC98 specific for human nucleolin (Chen et al., 1991). The mAb was revealed with goat anti-mouse immunoglobulin labeled with horseradish peroxidase (ECL; Amersham). In other experiments we used rabbit polyclonai antibodies which were revealed with donkey anti-rabbit immunoglobulin labeled with horseradish peroxidase (Amersham).

P. Two Dimensional Gel Isoelectric Focusing

Experimental conditions were as described previously (Krust et al., 1982). The concentration of ampholine (Pharmacia Biotech, Sweden) was at 2% of pH range 3-10. The pH gradient obtained by isoelectric focusing (first dimension) was from 4 to 7. The proteins were resolved in the second dimension on a 7.5% polyacrylamide-SDS gel.

Q. Complex Formation Between the Biotin-labeled 5[Kψ(CH₂N)PR]-TASP or the Biotin-labeled V3 Loop and the Cell Surface Expressed P95/nucleolin.

Twenty four hours after passaging, CEM cells were washed extensively with PBS before incubation (as 50×10⁶ cells per 300 μl of FACS buffer) at 4° C. for 30 min with the biotin-labeled 5[Kψ(CH₂N)PR]-TASP (10 μM) or the synthetic V3 loop (200 μM). Cells were then washed in FACS buffer (2×15 ml) and nucleus-free cell extracts were prepared using buffer E (150 ml). Such extracts were first diluted in PBS containing 1 mM EDTA (600 μl) before the addition of 100 μl avidin-agarose (ImmunoPure Immobilized Avidin from Pierce Chemical Company, U.S.A.) to capture the biotin-labeled TASP complexed to its cell surface ligand(s). These suspensions were incubated at 4° C. for 2 hr, and the avidin-agarose bound proteins were washed batchwise with PBS containing 1 mM EDTA (5×5 ml). Finally, the avidin-agarose pellet was resuspended in 100 ml of 2-fold concentrated electrophoresis sample buffer and heated at 95° C. for 5 min. The eluted proteins were analyzed by SDS/PAGE, and the V3 loop-BPs were revealed by ligand blotting using biotin,labeled 5[Kψ(CH₂N)PR]-TASP. The presence of nucleolin was also revealed by immunoblotting using murine monoclonal antibody CC98 or rabbit polyclonal antibodies against human nucleolin.

R. Complex Formation Between the Biotin-labeled 5[Kψ(CH₂N)PR]-TASP or the Biotin-labeled V3 Loop and Cellular Proteins

Cytoplasmic extracts were purified using affinity-matrix preparations composed of either the biotin-labeled V3 loop peptide or the biotin-labeled 5[Kψ(CH₂N)PR]-TASP. For this purpose, aliquots of 100 μl of avidin agarose in 200 μl of PBS/EDTA containing either the biotin-labeled 5[Kψ(CH₂N)PR]-TASP (20 μM) or the biotin-labeled V3 loop peptide (100 μM) were incubated (18 hours, 4° C.) before washing in PBS/EDTA (3×5 ml). Cell extracts (150 μl; corresponding to material from 50×10⁶ CEM cells) were first diluted in PBS/EDTA (600 μl) before adding to the different test tubes containing the washed affinity matrix preparations. After 2 hours of incubation at 4° C., the samples were processed as above and analyzed by SDS/PAGE. Under similar experimental conditions, the biotin-labeled 5[QN)PQ]-TASP construct (100 μM) was used as a control.

S. Cell Surface Labeing Using the Biotin-labeled Synthetic V3 Loop

CEM cells were washed in PBS, suspended in the FACS buffer (as 5×10⁵ cells per 100 ml) containing 25 mM of the biotin-labeled V3 loop, and incubated at 4° C. for 30 min. The cells were then washed twice with FACS buffer and fixed in 1% formaldehyde in FACS buffer. The biotin-label was then revealed by using streptavidin-FITC complex (Amersham). The fluorescence intensity was monitored by FACS analysis using Cell Quest™ Software (Beckton Dickinson).

T. Purification of the V3 Loop Binding Proteins and Microsequencing

Twenty four hours after passaging, CEM cells (2×10⁹ cells) were washed extensively with PBS before preparation of nuclear free extracts with buffer E (6 ml). All experimental procedures and centrifugations were carried out at 4° C. Such extracts were first centrifuged (1,000 g, 5 min) to remove nuclei, and the supernatant was then centriguged at 12,000 g for 15 min to remove mitochondria. Finally, the supernatant was centrifuged at 100,000 g for 30 min to remove ribosomes, and the supernatant S100 was stored at −80° C. For the preparation of the affinity matrix, 3 ml of avidin-agarose (ImmunoPure Immobilized Avidin from Pierce Chemical Company, U.S.A.) in PBS containing 1 mM EDTA (PBS/EDTA) was incubated in a total volume of 9 ml with biotin-labeled 5[Kψ(CH₂N)PR]-TASP (20 μM). After overnight incubation at 4° C., this suspension was washed batchwise with PBS/EDTA (2×30 ml). The 6 ml of cell extracts S100 preparation was first diluted in PBS/EDTA (24 ml) before addition to the affinity matrix. After incubation at 4° C. for 2 hours, proteins bound to the matrix were washed extensively with PBS/EDTA (6×125 ml). Elution of 5[Kψ(CH₂N)PR]-TASP bound proteins was carried out with 3×1.5 ml of the 1 M NaCl Elution buffer. The samples were dialyzed overnight against PBS/0.1 mM EDTA/1 mM PMSF and aliquots stored at −80° C. By this purification procedure, fractions 1 and 2 contained most of the V3 loop-BPs which were revealed by staining with Coomassie blue. These bands in ligand blotting type experiments bound biotin labeled 5[Kψ(CH₂N)PR]-TASP or biotin-labeled V3 loop. Fractions 1 and 2 were pooled and aliquots were stored at −80° C. The concentration of protein in this preparation of V3 loop-BPs was 100 μg/ml. This purification procedure was highly reproducible. In some experiments, such purified- preparations were concentrated using Centricon-10 or -100 filter sieve (Amicon, Inc. MA, USA) with a molecular weight cut-off 10,000 and 100,000 Daltons, respectively.

For the microsequencing of the purified V3 loop-BPs (P95/p60, P40, and P30), 3×250 ml aliquots of the purified preparation were analyzed by PAGE/SDS electophoresis using a 10% polyacrylamide slab gel. The different protein bands were visualized after a slight staining with Amido/Black. The respective bands wee excized from the gel and digested with endo-lysine C which cleaves peptides adjacent to lysine residues. The peptides were purified by an HPLC column (DEAE-C18) using a gradient of acetonitrile/trifluoroacetic acid 0.1%. The microsequencing was carried out by the Protein-Sequencing laboratory at Institut Pasteur.

U. Purification of the Cell Surface Expressed P95 for Microsequencing

Twenty four hours after passaging, CEM cells (10⁹ cells) were washed extensively with PBS before incubation (in 10 ml of FACS buffer) at 40° C. for 30 min with 15 μM of the biotin-labeled 5[Kψ(CH₂N)PR]-TASP. Cells were then washed in FACS buffer (2×100 ml) and nucleus-free cell extracts were prepared using buffer E (3 ml). Such extracts were first diluted in PBS (12 ml) before the addition of 1 ml avidin-agarose to capture the biotin-labeled TASP complexed to P95. These suspension was incubated at 4° C. for 2 hr, and then washed batchwise with PBS (6×60 ml). Finally, the avidin-agarose pellet was resuspended in 2 ml of 2-fold concentrated electrophoresis sample buffer and heated at 95° C. for 5 min. An aliquot (20 μl) of the eluted fraction was analyzed by SDS/PAGE to monitor the purity of the preparation by staining the gel with Coomassie blue. In addition, P95 was revealed by ligand blotting using the biotin-labeled 5[Kψ(CH₂N)PR]-TASP.

For the microsequencing the NH₂-terminal sequence of the cell surface expressed P95, 3×250 μl aliquots of the purified preparation were analyzed by PAGE/SDS electophoresis using a 7.5% polyacrylamide slab gel. The P95 band was transferred to a PVDF membrane before microsequencing the NH₂-terminal (carried out by the Protein-Sequencing laboratory at Institut Pasteur).

V. Production of Polyclonal Antibodies Against P95/nucleolin, P40/PHAP II, P30/PHAP I, CXCR4 and the Purified Preparation of the V3 Loop-BPs

Peptides corresponding to the NH₂-terminal sequences of P95/nucleolin, P40/PHAP II, and P30/PHAP I were synthesized according to conventional methods. The following peptides were synthesized (N for the NH₂-terminal sequence; I for internal sequence; C for an additional cysteine residue): P95N, residues 1-26(C) of nucleolin; P95I, residues (C)266-292 of nucleolin; P40N, residues 1-23(C) of PHAP II; P40I, residues (C)211-230 of PHAP II; P30N, residues 1-20(C) of PHAP I; P30I, residues 2949(C). The peptides were conjugated to ovalbumin through the additional cysteine residues. Two rabbits (New Zealand, female, 2 months) were immunized at two weeks interval by five intramuscular injections with the coupled material corresponding to 150 μg of each of the peptide. The first injection was with complete Freund's adjuvant (CFA) while the following injcections were with incomplete Freund's adjuvant (IFA). After the third and the fourth injections, rabbit antisera were titrated for the production of antibodies by monitoring reactivity with the respective peptide and also with the purified preparation of the V3 loop-BPs. Using a similar protocol, polyclonal antibodies were also produced in rabbits against CXCR4 by immunization with a synthetic peptide corresponding to the NH₂-terminal amino acids 1-27(C) conjugated to ovalbumin. In another protocol, a rabbit was immunized against the V3 loop-BPs by five subcutaneous injections of the purified preparation of the V3 loop-BPs (100 μg) along with CFA. The production of rabbit antibodies against a synthetic peptide corresponding to the first 13 amino acids of histone H2B, and to an internal peptide 40-55 of U₁ small nuclear ribonucleolprotein (RNP) C were as previously described (Benkirane et al., 1995).

To test the effect of antibodies against HIV infection, sera were diluted with an equal volume of PBS and sterilized by filtration through a Millipore filtre (0.22 μm).

W. Purification of Polyclonal Antibodies Against P95/nucleolin, P40/PHAP II, and P30/PHAP I

Rabbit polyclonal antibodies against nucleolin, PHAP II and PHAP I, were purified by virtue of their affinity towards the corresponding synthetic peptide. For this purpose, affinity columns were prepared by coupling 4 mg of each respective peptide to a 1 ml Hitrap colomn (Parmacia) under the conditions as recommended by the manufacturer. Four ml of rabbit antisera were then purified on such colums and the bound immunoglobulins were eluted by 0.2 M glycine at pH 3. Purification of immunogobulins by affinity chromatography using protein A sepharose (Pharmacia) was according to the methods described by the manufacturer.

X. Enzyme Linked Immunoaborbent Assays (ELISA) to Show the Binding of gp120 to V3 Loop-BPs

The ELISA procedure was as described previously (Benkirane et al., 1995) with slight modifications. Briefly, microtiter plates (Falcon) were coated overnight at 37° C. with different concentrations (0 to 200 ng/ml) of the V3 loop-BPs preparation in a solution of 0.05 M carbonate buffer, pH 9.6. After 3 washings of the microtiter plates with PBS containing 0.05% Tween (PBS-T), the plates were incubated (1 hour at 37° C.) in PBS-T containing 10 mg/ml bovine serum albumin (PBS-T-BSA) with gp120 (1 ng/ml), or gp41 (2 ng/ml), or histone H3 (5 ng/ml). After 3 washings with PBS-T, the following monoclonal antibodies in PBS-T-BSA were added: mAb 110-D against gp120 (1 μg/ml), mAb 41-A against gp41 (1 μg/ml), and a mAb specific for histone H3 (2 μg/ml; Benkirane et al., 1995). In these experiments, as a control mAb, we used mAb OKT4A (1 μg/ml) specific for human CD4. After incubation for 1 hour at 37° C., the plates were washed extensively in PBS-T, and positive reactions were detected using AffiniPure goat anti-mouse IgG (H+L) conjugated to horseradish peroxidase (Nordic, Tilburg, The Netherlands; working dilution 1:5,000). After 30 min of incubation and washing with PBS-T, the final reaction was visualized by incubation with 3,3′,5,5′-tetramethylbenzidine in the presence of H₂O₂ (Briand et al., 1992). The resulting absorbance was measured at 450 nm. An optical density (OD) value less than 0.3 was considered as background, therefore negative.

Y. Biosensor Measurements

For real time binding experiments, a BIAcore biosensor system (Pharmacia biosensor, AB, Uppsala, Sweden) was used. Experimental procedures were as described previously (Benkirane et al., 1995; Stemmer et al., 1996). Reagents including sensor chip CM5, surfactant P20, and coupling kit containing N-hydroxysuccinimide (NHS), N-ethyl-N′-(3-dimethylaminopropyl) carbodiimide (EDC) and ethanolamine HCl were obtained from Pharmacia Biosensor AB. To immobilize the V3 loop-BPs to the sensor chip, the carboxyl-dextran matrix was first activated with 0.2 M EDC and 0.05 M NHS. V3 loop-BPs were immobilized by adding on the activated dextran matrix (163.4 resonance units, RU). The peptides 5[Kψ(CH₂N)PR]-TASP and 5(KPR)-TASP, or the gp120 preparation were subsequently injected at a constant flow rate of 5 μl/min during 7 min at 25° C. and report points for calculation were taken every 10s during 5 min, starting 1.5 min after the end of peptide or gp120 injection. 6-8 concentrations of each peptide/protein ranging from 5 to 600 nM in HSB, pH 7.4 (HSB: 10 mM HEPES, 150 mM NaCl, 3.4 mM EDTA, 0.05% surfactant P20) were used in each test. Theory of kinetic measurements using the BIAcore biosensor system has been described previously (Saunal et al., 1996).

Z. The Production of HIV-1 Pseudotyped Mo-MLV Virus

Hela cells were cotransfected by electroporation with plasmids pNL4-3 defective in env gene (Borman et al., 1995) and pE4070A expressing amphotrope envelope glycoproteins of Mo-MLV (Battini et al., 1996). Electroporation (Schwartz et al., 1996) was performed at 200 V, 960 μF, using a 4 mm wide cuvettes in a BIO-Rad Gene Pulser. The pseudotyped virus was recovered from the culture supernatant after 48 hours of culturing. Plasmids were kindly provided by Dr. S. Le Gall (Institut Pasteur, Paris).

### EXAMPLES

### Example 1

5[Kψ(CH₂N)PR]-TASP Blocks HIV Entry by Inhibiting the Membrane Fusion Process

We have recently reported that 5[Kψ(CH₂N)PR]-TASP and related TASP-inhibitors block HIV entry and thus infection (7). Such inhibition of viral entry could be demonstrated by different experimental approaches. For example, HIV entry monitored by the intracellular concentration of p24 (HIV-1 major core protein) following 1 hour incubation of CEM cells with the virus, is inhibited almost completely in the pesence of 5-10 μM of 5[Kψ(CH₂N)PR]-TASP. Similarly, viral entry monitored by the b-galactosidase activity in HeLa/CD4⁺ cells expressing the bacterial lacZ gene placed under the control of the HIV-1 LTR, is inhibited by at least 90% at similar concentrations of 5[Kψ(CH₂N)PR]-TASP (not shown; as described in Callebaut et al., 1996). Otherwise, addition of 5[Kψ(CH₂N)PR]-TASP after the viral entry process does not affect virus infection, monitored by the production of virus at 4 days post-infection (p.i; as shown in FIG. 2 in Callebaut et al., 1996). Here we further investigated the timing of the inhibitory effect of 5[Kψ(CH₂N)PR]-TASP during the HIV entry process. Addition of 5 mM 5[Kψ(CH₂N)PR]-TASP to cells one hour before or together with the virus, resulted in more than 90% inhibition of virus production. On the other hand, addition of 5[Kψ(CH₂N)PR]-TASP at 2, and 4 hours p.i. reduced its inhibitory effect, and when added at 8 hours then there was almost no effect (not shown, experimental procedures as in Callebaut et al., 1996). The period of 8 hours is necessary for HIV-1 entry in at least 90% of target cells in the CEM cell culture (Laurent-Crawford et al., 1993). The effect of 5[Kψ(CH₂N)PR]-TASP on the viral entry process is most probably the consequence of the inhibition of the membrane fusion process. In accord with this, we have previously provided evidence to demonstrate that 5[Kψ(CH₂N)PR]-TASP blocks efficiently the gp120/gp41 mediated membrane fusion observed in cocultures of chronically HIV-1 infected cells with uninfected CD4⁺ cells (see FIG. 6 in Callebaut et al., 1996).

In order to further investigate the mechanism of inhibition of HIV entry, we studied the effect of 5[Kψ(CH₂N)PR]-TASP on the binding of gp120 and HIV-1 particles to CD4⁺ CEM cells. In the first set of experiments, cells were incubated with different concentrations of 5[Kψ(CH₂N)PR]-TASP before further incubation with ¹²⁵I-labeled gp120. Under these experimental conditions, the binding of ¹²⁵I-labeled gp120 was specific, since it was inhibited by anti-CD4 mAb OKT4A, known to block the binding of gp120 to the CD4 receptor (Krust et al., 1993; Mizukami et al., 1988). The binding of gp120 was not affected at 20 mM of 5[Kψ(CH₂N)PR]-TASP, whereas there was a slight inhibition of binding at higher concentrations (FIG. 1A +L). To investigate the binding of virus to cells, CEM cells were incubated with HIV-1 particles for 1 hour. Cells were then washed extensively and the bound virus (including that which was entered into cells) was estimated by the concentration of p24 in cell lysates. As the binding of gp120, the binding of virus was specific since it was inhibited (75%) by mAb OKT4A. Interestingly, at 10 mM of 5[Kψ(CH₂N)PR]-TASP the binding of HIV particles was also inhibited (78%) at a similar extend as that exerted by mAb OKT4A alone, or when 5[Kψ(CH₂N)PR]-TASP was used combined with mAb OKT4A (FIG. 1B +L). Thus. the 22% residual binding in the presence of 5[Kψ(CH₂N)PR]-TASP should represent unspecific binding. It is plausible therefore to consider that 5[Kψ(CH₂N)PR]-TASP inhibits the gp120/gp41 mediated membane fusion by affecting the interaction of this complex with CD4⁺ cells.

The discrepancy between the binding results of soluble gp120 and viral particles to CEM cells (FIG. 1 +L), indicate that gp120 complexed to gp41 on the surface of viral particles does not have the same conformational restrictions as the soluble gp120. Thus, experiments using soluble gp120 should be interpreted cautiously.

### Example 2

Specific Binding of 5[Kψ(CH₂N)PR]-TASP to a Cell-surface Protein

By FACS analysis, here we show that the FITC-labeled 5[Kψ(CH₂N)PR]-TASP binds different types of human cells, such as CD4⁺ T cell lines CEM and MOLT4, and PHA-stimulated PBMC, and as well as the CD4⁻ HeLa cells (FIG. 2 +L). In all of these cells, the cell-surface binding of FITC-labeled 5[Kψ(CH₂N)PR]-TASP was specific since it was prevented by the unlabeled 5[Kψ(CH₂N)PR]-TASP molecule (FIG. 2 +L, sections 1, 2, 5 and 6). Interestingly, cell-surface binding of FITC-labeled 5[Kψ(CH₂N)PR]-TASP was prevented by all TASP constructs active against HIV-infection (not shown), such as the 5[KPR]-TASP (FIG. 2 +L, section 4 ) but not by constructs which were inactive (Callebaut et al., 1996), such as 5[KGQ]-TASP (. 2 +L, section 3). These results are consistent with the suggestion that the different anti-HIV TASP constructs interact with the same cell-surface component since they have the capacity to competitively block the binding of the FITC-labeled 5[Kψ(CH₂N)PR]-TASP to cells.

In an attempt to determine the proteinaceous nature of the cell-surface component to which peptide-TASP inhibitors bind, cell-surface labeling of MOLT4 cells with FITC-labeled 5[Kψ(CH₂N)PR]-TASP was investigated after proteolysis with trypsin or proteinase K. As controls for proteolysis we investigated the expression of cell-surface CD26 with the mAb Ta1 and CD4 with mAbs OKT4A and OKT4. The anti-CD4 antibodies recognize different epitopes on the CD4 receptor: mAb OKT4A is against an epitope in the NH₂-terminal extracellular domain, whereas the epitope recognized by Mab OKT4 seems to be close to the cell membrane because it is resistant to trpsin treatment (Mikuzami et al., 1988; Rao et al., 1983). As it was expected, trypsin treatment abolished the OKT4A but not OKT4 epitope to be recognized by their respective antibody. Under the same experimental conditions, trypsin treatment did not affect the binding of 5[Kψ(CH₂N)PR]-TASP nor mAb Ta1. However, the binding of the TASP inhibitor was abolished by proteinase K treatment, which even affected the OKT4 epitope. On the other hand, the Ta1 epitope remained resistant to proteinase K (FIG. 3 +L). Such results were reproducibly observed in several experiments summarized in Table 1; i.e., the binding of the FITC-labeled 5[Kψ(CH₂N)PR]-TASP was not affected by trypsin treatment, whereas it was abolished by proteinase K or pronase E treatment, which also abolished the OKT4A epitope in CD4. Once again however, the Ta1 epitope remained resistant to proteinase K and to pronase E (Table 1). This latter resistance reveals an intriguing nature of the cell-surface expressed CD26 to resist proteolysis, which we have reported recently (Jacotot et al., 1996). Taken together, our results indicate that the 5[Kψ(CH₂N)PR]-TASP-binding entity on cells is most likely a protein which is resistant to trypsin but sensititive to proteinase K and pronase E. Furthermore, such a potential TASP-binding protein does not seem to be CD4 nor CD26.

### Example 3

The Higher Affinity of 5[Kψ(CH₂N)PR]-TASP Compared to 5[KPR]-TASP to Bind the Cell-surface

5[Kψ(CH₂N)PR]-TASP and its non-reduced counterpart 5[KPR]-TASP are potent inhibitors of HIV-1 entry and infection (7), with IC₅₀ values in CEM cells as 0.5 and 5 μM, respectively (Table 2). For further characterization of the TASP-inhibitor binding protein on the cell-surface, biotin-labeled TASP inhibitors along with control TASP constructs (5[QPQ]- and 5[KGQ]-TASP) that lack activity against HIV infection, were investigated by FACS analysis (Table 2; FIG. 4 +L). Clearly, no cell-surface labeling occured with control TASP molecules. On the other hand, both biotin-labeled 5[Kψ(CH₂N)PR]- and 5[KPR]-TASP molecules were found to bind CEM cells with 50% effective-binding concentration values 0.15 and 3.5 μM, respectively. These results illustrate that 5[Kψ(CH₂N)PR]-TASP manifests, at least, 10-fold higher activity compared to its non-reduced TASP-counterpart, for both the inhibition of HIV infection and the affinity to bind the cell-surface ligand. This latter favors the hypothesis that inhibition of HIV infection is a consequence of specific binding of the TASP inhibitor to its cell-surface ligand.

5[KPR]- and 5[Kψ(CH₂N)PR]-TASP are stable in the FACS buffer. However, when incubated in serum from fetal calf or from an individual seropositive for HIV-1, 5[KPR]-TASP rapidly loses its activity (probably due to proteolysis) with a half-life of about 1 hour. In contrast, 5[Kψ(CH₂N)PR]-TASP retains more than 80% of its activity after 18 hours of incubation at 37° C. (Table 2).

### Example 4

Identification of a 95 kDa Protein in Cell Extracts by Ligand Blotting Using Biotin-labeled 5[Kψ(CH₂N)PR]-TASP.

Crude extracts from CEM cells were assayed by ligand blotting using 5 μM of biotin-labeled 5[Kψ(CH₂N)PR]-TASP. A single protein band, migrating just underneath the 97 kDa molecular weight protein marker was revealed; this 5[Kψ(CH₂N)PR]-TASP binding protein is referred to as P95 (FIG. 5 +L). The binding was specific, since it was abolished in the presence of excess unlabeled 50 mM of 5[Kψ(CH₂N)PR]-TASP (FIG. 5B +L), whereas, 50 or 100 μM of 5[QPQ]- and 5[KGQ]-TASP had no effect (as in FIG. 5A +L). Under similar experimental conditions, biotin-labeled 5[KPR]-TASP construct but not 5[QPQ]- or 5[KGQ]-TASP constructs revealed P95 (data not shown).

In order to determine the molecular mass of P95 under nondenaturing conditions, cell extracts were subjected to gel filtration chromatography and fractions were analyzed by ligand blotting. The 5[Kψ(CH₂N)PR]-TASP binding protein eluted as a protein of molecular mass between 90 to 100 kDa (data not shown; the experimental conditions were as described in “Materials and Methods”). A small amount of an 80 kDa protein was detectable in fractions containing P95. However, as the elution profile of this 80 kDa protein was identical to that of P95, then the 80 kDa protein is most probably a degradation product of P95 which could have been generated even during SDS/PAGE. Indeed, if the degradation had occured before gel filtration, then the elution of the 80 kDa protein would have been delayed in relation to that of P95.

### Example 5

Stable Complex Formation Between 5[Kψ(CH₂N)PR]-TASP and the Cell-surface Expressed P95.

CEM cells preincubated with biotin-labeled control and anti-HIV TASP constructs, were washed extensively and cell extracts were purified by avidin-agarose in order to isolate any potential complexes formed between the biotin-labeled TASP constructs and cell-surface proteins (“Materials and Methods”). The presence of P95 in such purified preparations was then revealed by ligand blotting using biotin-labeled 5[Kψ(CH₂N)PR]-TASP. Under these experimental conditions, P95 was recovered when cells were preincubated with either biotin-labeled 5[KPR]- or 5[Kψ(CH₂N)PR]-TASP (FIG. 6 +L, lanes 4/5) but not with biotin-labeled control TASP constructs, 5[KGQ]- or 5[QPQ]-TASP (FIG. 6 +L, lanes 2/3). Consistent with the higher affinity of 5[Kψ(CH₂N)PR]-TASP to bind its cell-surface ligand compared to 5[KPR]-TASP construct (Table 2, 4 +L), almost 2-fold higher amount of cell-surface P95 was recovered by the reduced compared to the unreduced TASP inhibitor (FIG. 6 +L, lanes 4 and 5). The isolation of cell-surface P95 by preincubation of cells with biotin-labeled 5[Kψ(CH₂N)PR]-TASP was specific, since it was completely abolished in the presence of excess of unlabeled 5[Kψ(CH₂N)PR]-TASP during the preincubation period (FIG. 6 +L, compare lanes 5 and 7). Consistent with its lower affinity to bind P95, excess of 5[KPR]-TASP abolished about 40 to 50% (FIG. 6 +L, compare lanes 5 and 6). On the other hand, the control 5[QPQ]-TASP construct had no effect (FIG. 6 +L, lane 8).

These results therefore, demonstrated that the biotin-labeled TASP inhibitors bind to the cell-surface expressed P95, and that this complex is stable, since it could be isolated by the strong affinity of biotin to bind avidin. The 5[Kψ(CH₂N)PR]-TASP/P95 complex is highly stable at physiological salt concentrations but dissociates at concentrations of NaCl>200 mM (data not shown). To confirm that P95 isolated under experimental conditions described in FIG. 6 was indeed from the cell surface, CEM cells were iodinated to label cell-surface proteins, before incubation with biotin-labeled 5[QPQ]- or 5[Kψ(CH₂N)PR]-TASP constructs (FIG. 7 +L). Cells were then washed, extracted, and the biotin-labeled TASP-protein complexes were isolated by purification using avidin-agarose. By ligand blotting, we first demonstrated that when cells were preincubated with biotin-labeled 5[Kψ(CH₂N)PR]-TASP but not 5(QPQ)TASP, then P95 was recovered after purification (FIG. 7B +L, lanes 2 and 3). The hypothetical degradation product of P95, the 80 kDa protein was once again detected along P95 (FIG. 7B +L, lane 3). Analysis of the purified preparation by SDS/PAGE and autoradiography, revealed that both P95 and the 80 kDa by product were labeled with ¹²⁵I, and which were isolated specifically when cells were preincubated with the biotin-labeled 5[Kψ(CH₂N)PR]-TASP construct (FIG. 7A +L, lane 3). An highly ¹²⁵I-labeled 140 kDa protein was found to bind avidin-agarose independent of the biotin-labeled TASP constructs (FIG. 7A +L, lanes 1-3). The identity of this 140 kDa protein is not known. The isolation of ¹²⁵I-labeled P95 from labeled cell-surface proteins was also demonstrated by preincubation of cells with biotin-labeled 5[KPR]-TASP (Data not shown, similar to those in FIG. 7 +L). Consequently, the results of FIG. 7 provide further confirmation along those shown in 6 +L, that a significant proportion of P95 is expressed on the cell surface, and that this protein interacts specifically with 5[Kψ(CH₂N)PR]-TASP and related anti-HIV TASP constructs.

Comparison of the estimated amount of the P95 found in crude cell extracts (6 +L, lane 1) with that isolated from the cell surface (6 +L, lane 5), suggested that cell-surface P95 could represent less than 20% of the total cellular P95. This is consistent with the low amount of P95 that we could recover in plasma membrane preparations (“Materials and Methods”) compared to that found in cytoplasmic extracts (not shown).

### Example 6

The Presence of a P95 Like Protein in Different Types of Human and Murine Cells

In ligand blotting type experiments (not shown, experimental procedures as in FIGS. 5 and 6 +L) using cell extracts and the biotin-labeled 5[Kψ(CH₂N)PR]-TASP, we could demonstrate the expression of a 95 kDa protein in different types of human (MOLT4, Jurkat, HeLa, Daudi) and murine (NIH/3T3, L929, hybridoma) cells, similar to P95 in CEM cells.

### Example 7

5[Kψ(CH₂N)PR]-TASP Does Not Interact with HIV Proteins

Several observations indicated that 5[Kψ(CH₂N)PR]-TASP does not interact with HIV-1 envelope gp120/gp41 nor with other viral proteins (not shown). Firstly, by FACS analysis, we demonstrated that FITC- or biotin-labeled 5[Kψ(CH₂N)PR]-TASP does not react with the cell-surface gp120/gp41 complex expressed by chronically HIV-1-infected cells. Secondly, HIV-1 particles were not retained on an affinity column constructed with the biotin-labeled 5[Kψ(CH₂N)PR]-TASP bound to avidin-agarose. Thirdly, ¹²⁵I-labeled gp120 (as in 1 +LA) or metabolically ³⁵S-Methionine-labeled HIV-1 proteins (as described by Laurent-Crawford et al., 1993) were not retained on the 5[Kψ(CH₂N)PR ]-TASP-affinity column. Finally, in ligand blotting type experiments using extracts from concentrated HIV-1 Lai particles, we demonstrated that the biotin-labeled 5[Kψ(CH₂N)PR]-TASP does not interact with any of the HIV proteins.

### Example 8

Binding of 5[Kψ(CH₂N)PR]-TASP and the V3 Loop to the Cell Surface Expressed P95.

By FACS analysis, we have previously shown that 5[Kψ(CH₂N)PR]-TASP binds specifically to different types of human cell lines and PHA-stimulated PBMC. Furthermore, in ligand blotting type expresiments using crude cell extracts and the biotin-labeled 5[Kψ(CH₂N)PR]-TASP, we have identified a 95 kDa protein which interacts specifically with TASP constructs active against HIV entry. This 95 kDa protein (P95) is expressed on the cell surface, since surface iodination of cells resulted in its labeling, and moreover, following incubation of cells with the biotin-labeled 5[Kψ(CH₂N)PR]-TASP, the P95/TASP complex was recovered by affinity chromatography using avidin-agarose (Callebaut et al., 1997b; FIG. 8 +L).

A biotin-labeled V3 loop peptide, corresponding to the amino acid sequence found in the gp120 of HIV-1 Lai isolate, was synthesized in order to investigate the interaction of the V3 loop with the cell surface P95. By FACS analysis, we first demonstrated that the biotin-labeled V3 loop peptide binds to the surface of CEM cells in a dose dependent manner (not shown). This binding was significantly reduced in the presence of unlabeled 5[Kψ(CH₂N)PR]-TASP (FIG. 8A +L), suggesting that the V3 loop and 5[Kψ(CH₂N)PR]-TASP interact with a similar cell surface ligand. Secondly, to demonstrate complex formation with cell surface P95, CEM cells in FACS buffer were incubated with either the biotin-labeled 5[Kψ(CH₂N)PR]-TASP, or the biotin-labeled V3 loop peptide, or with the control construct, the biotin-labeled 5[QPQ]-TASP. Cells were then washed extensively and extracts were purified using avidin-agarose to capture the biotin-labeled V3 loop/5[Kψ(CH₂N)PR]-TASP complexed to the cell surface P95. The samples were analyzed by SDS/PAGE, and the V3 loop binding proteins were revealed by ligand blotting using biotin-labeled 5[Kψ(CH₂N)PR]-TASP (FIG. 8B +L). The results demonstrate that the V3 loop binds and forms a stable complex with cell surface P95. Consistent with the suggestion that the V3 loop peptide and 5[Kψ(CH₂N)PR]-TASP bind to the same cell surface protein, the binding of the biotin-labeled V3 loop peptide to P95 was significantly reduced in the presence of excess unlabeled 5[Kψ(CH₂N)PR]-TASP (FIG. 8B +L).

### Example 9

Purification of Several Proteins from Crude Cell Extracts Using the Biotin-labeled 5[Kψ(CH₂N)PR]-TASP.

Several experiments were carried out to optimise the experimental conditions for the purification of 5[Kψ(CH₂N)PR]-TASP binding proteins from crude cell extracts using the biotin-labeled TASP construct. After the recovery and washing on avidin-agarose, the 5[Kψ(CH₂N)PR]-TASP bound proteins were revealed by ligand blotting using the biotin-labeled 5[Kψ(CH₂N)PR]-TASP. By this process, we purified several proteins including P95 (shown below). No proteins were recovered by the avidin-agarose in the absence of the biotin-labeled 5[Kψ(CH₂N)PR]-TASP or when cell extracts were incubated with the biotin-labeled control 5[QPQ]-TASP construct (not shown). Thus the biotin-labeled 5[Kψ(CH₂N)PR]-TASP coupled to avidin-agarose represents an efficient affinity matrix for the purification of 5[Kψ(CH₂N)PR]-TASP binding proteins.

Large quantities of purified proteins were prepared by using extracts from 10⁹ cels (“Experimental Procedures). FIG. 9 shows the profile of the purified proteins revealed by staining the PAGEISDS gel with Coomassie blue, and by ligand blotting using either the biotin-labeled 5[Kψ(CH₂N)PR]-TASP or the biotin-labeled V3 loop peptide. By this experimental procedure four major proteins of 95, 60, 40, and 30 kDa (P95, p60, P40, and P30, respectively) were purified. Each one of these proteins binds 5[Kψ(CH₂N)PR]-TASP and the V3 loop peptide, thus pointing out that these purified proteins are V3 loop binding proteins (hereafter referred to as the V3 loop-BPs). Consistent with this, P95, p60, P40, and P30 could also be purified using crude extracts and an affinity matrix containing the synthetic V3 loop peptide (shown below).

It should be emphasized that both the biotin-labeled 5[Kψ(CH₂N)PR]-TASP and the V3 loop peptide recovered only P95 from the cell surface, whereas from cell extracts they purified P95, p60, P40, and P30 (FIGS. 8 and 9 +L). This latter could probably be due to a higher affinity of 5[Kψ(CH₂N)PR]-TASP and the V3 loop peptide towards P95 compared to the other proteins. Consequently, the recovery of the complexes formed between cell surface P95 and the 5[Kψ(CH₂N)PR]-TASP construct/the V3 loop peptide could be efficient because of the stability of this complex (FIG. 8B +L).

### Example 10

Identification of V3 Loop-BPs: P95/nucleolin; P40/PHAP II, and P30/PHAP I.

The four proteins purified from cell extracts (FIG. 9 +L) were analyzed by microsequencing after digestion with endo-lysine C which cleaves peptides adjacent to lysine residues (“Experimental Procedures”). The peptides were purified by an HPLC column and some of the peaks were processed for microsequencing. The amino acid sequences obtained from the different peptides indicated that P95 is nucleolin (Srivastava et al., 1989), P40 is PHAP II (Vaesen et al., 1994), P30 is PHAP I (Vaesen et al., 1994), whereas p60 should correspond to a partiallly degraded product of nucleolin (Table 3). To verify the NH₂-terminal sequence of the cell surface expressed P95 (FIG. 8B +L), a purified sample of cell surface P95 was prepared and processed for microsequencing as described in “Experimental Procedures”. The 15 amino acid sequence obtained was found to be 100% identical with that of the NH₂-terminal sequence of human nucleolin (Srivastava et al., 1989).

Nucleolin is the major non-histone protein of the nucleolus which has been suggested to shuttle between nucleus and cytoplasm (Borer et al., 1989). The deduced amino acid sequence of nucleolin reveals at its NH₂-terminal half, three stretches of about 26 amino acids each, domains which contain more than 85% either aspartate or glutamate (Srivastava et al., 1989). Although primarily localized in the cell nucleoli, nucleolin or nucleolin-like proteins have been reported to be expressed on the cell surface (Pfeifle and Anderer, 1983; Kleinman et al., 1991; Jordan et al., 1994; Krantz et al., 1995). In the literature, the apparent molecular weight of nucleolin after PAGE/SDS has been referred to as 92 to 110 kDa, and several reports have emphasized the susceptibility of nucleolin to partial degradation (Bugler et al., 1982; Fang and Yeh, 1993 Exp. Cell Res. 208, 48-53, 1993). Microsequencing of the NH₂-terminus and two internal peptides of P95 revealed 100% identity with the corresponding regions in the human nucleolin sequence (Table 3). Attempts to microsequence the NH₂-terminus of p60 failed several times. However, we were able to obtain the sequence from several internal peptides of p60, which were found to be homologous to the nucleolin sequence. Interestingly, the sequence of the peptide in peak 18 of p60, suggests that p60 should correspond to the COOH-terminal portion of nucleolin.

PHAP I and PHAP II had been isolated as putative HLA Class II associated proteins because of their affinity to bind specifically to a synthetic peptide corresponding to the cytoplasmic COOH-terminal domain of MHC class II DR2a but not to DR2b chain (Vaesen et al., 1994). The predominant structural feature of both PHAP I and PHAP II is a long stretch of acidic amino acids composed of aspartate and glutamate residues at their C-Terminal ends (Vaesen et al., 1994). Microsequencing several peptides from P30 and P40 revealed their identity as PHAP I and PHAP II, respectively (Table 3). In addition to the amino acid sequence homology, the migration profile of P30 and P40 observed in PAGE/SDS, corresponded well with the reported profile of PHAP I and PHAP II (Vaesen et al., 1994). It should be noted that the 14 amino acid sequence of the peak 29 from P30, (K)KLELSENRIFGGL (Table 3) is homologous to the amino acid fragment KKLELSDNRVSGGL at position 67 to 80 in PHAP I. The differences between the deduced amino acid sequence of PHAP I and the sequence obtained by microsequencing are D/ to E, V to I, and S to F. Considering the genetic code for these amino acids, a single error in the nucleotide sequence might have accounted for this difference. As the PHAP I cDNA was obtained after PCR amplification using degenerated primers, it is plausible to suggest that some errors might have been generated during the amplification process (Vaesen et al., 1994).

The V3 loop BPs were therefore identified as P95/nucleolin, P40/PHAP II, and P30/PHAP I (Table3). The common feature between these three proteins is their polyanionic regions in virtue of the expression of the extended stretches of acidic amino acids. Such domains are probably responsible for the interaction with the V3 loop peptide or with the 5[Kψ(CH₂N)PR]-TASP pseudopeptide. In this respect, it is worthwhile to mention here that polyanions have been shown to be potent inhibitors of HIV entry through their potentail capacity to interact with the V3 loop domain (Javaherian and McDanal, 1995; Leydet et al., 1996).

### Example 11

The V3 Loop-BPs, Nucleolin, PHAP II and PHAP I Bind the Pseudopeptide 5[Kψ(CH₂N)PR]-TASP and the Synthetic V3 Loop Peptide

In order to confirm the identity of P95, P40, and P30 (Table 3), rabbit antibodies were generated against synthetic peptides corresponding to the NH₂-terminal and internal sequences of nucleolin, PHAP II and PHAP I (“Experimental Procedures”). Such antibodies were shown to be highly specific, since by immunoblotting each rabbit antiserum reacted only with the protein corresponding to the peptide which was used for immunization (1 +L0). Accordingly, in crude CEM cells, serum raised against peptides corresponding to nucleolin, PHAP II, PHAP I, revealed 95, 40, and 30 kDa bands, respectively (1 +L0, lanes Extract). The preimmune sera from the different rabbits did not show any signal (data not shown). In the purified preparations by the affinity matrix containing either 5[Kψ(CH₂N)PR]-TASP or the V3 loop peptide, the antibodies confirmed that nucleolin, PHAP II, PHAP I, indeed bind to the V3 loop (FIG. 9 +L, lanes V3 loop). The monoclonal antibody CC98 specific to human nucleolin, reacted only with P95 in crude extracts, whereas in the purified samples it reacted with P95 and p60, thus further confirming that p60 is a partial degradation product of nucleolin generated during the purification process. Interestingly, serum against the NH₂-terminal peptide of nucleolin did not react with p60 (1 +L0, Panel a-nucleolin), consistent with the suggestion that p60 should correspond to the COOH-terminal portion of nucleolin. Rabbit antiserum raised against the peptide corresponding to the NH₂-terminal domain of CXCR4 reacted with a 50 kDa protein in crude cell extracts, but it did not generate a signal in the purified fractions corresponding to the V3 loop BPs (1 +L0, Panel a-CXCR4). Similarly, rabbit antiserum against human CD4 reacted with the 60 kDa CD4 protein, but it did not reveal a signal in the purified preparations of the V3 loop-BPs (1 +L0, Panel a-CD4).

On the whole, these experiments confirm that 5[Kψ(CH₂N)PR]-TASP and the V3 loop peptide bind a similar pattern of proteins, P95, P40, and P30, which were referred to as the V3 loop BPs. Futhermore, they provide further confirmation concerning the identity of the V3 loop-BPs as being nucleolin, PHAP II and PHAP I, respectively. Moreover, they point out that CXCR4, the cofactor of CD4 required for the entry of lymphotropic HIV-1 isolates, does not bind the V3 loop.

### Example 12

Cell Surface Expressed Nucleolin/P95 Could be Differentiated from that of the Nucleus

By immunoblotting using the specific antibodies raised in rabbits, nucleolin was found in both the cytoplasmic and nuclear fraction of CEM cells as it was expected. On the other hand, PHAP I and PHAP II were detectable only in the cytoplasmic fraction (1 +L1). Further characterisarion of nucleolin in the cytoplasm and nuclear compatment was carried out by two dimensional gel isoelectric focusing experiments, along with the P95/nucleolin sample purified from the cell surface (“Experimental Procedures”). The nucleolin was revealed by immunoblotting using rabbit polyclonal antibodies against the purified human nucleolin (1 +L2). These experiments revealed that nuclear nucleolin is distinct from that of cytoplasmic and cell surface expressed nucleolin. Indeed, nuclear nucleolin was found to be composed of several related species with pI values between the pH 4.5 to 5.5 (1 +L2, Panel B). On the other hand, the cell surface expressed and cytoplasmic nucleolin has a pI value at about pH 4.5 (1 +L2, Panels A and C). Similar results were reproducibly obtained in different independent experiments. This difference between the cell surface and nuclear nucleolin might be the consequence of post-translational modifications, which determine the targeting of nucleolin towards the nucleus or the cell surface. In these experiments, an 80 kDa partial degradation product of P95/nucleolin was detected, in the samples of the cell surface expressed preparation of P95 and in the crude cytoplasmic extracts (1 +L2, Panels A and C).

### Example 13

Cell Surface Expression of P95/Nucleolin in Different Cell Lines

In order to investigate the cell surface expression of nucleolin, different human (HeLa, RD, Daudi, MOLT4, CEM, U937, and Jurkat) and murine (L929, T54, and T54/W12) cell lines were investigated. For this purpose, cell surface nucleolin was prepared by incubating intact cells with the biotin-labeled 5[Kψ(CH₂N)PR]-TASP and the recovery of the complex on avidin-agarose. The samples were analyzed by immunoblotting using rabbit polyclonal antibodies against the purified human nucleolin (1 +L3A). The results indicated that cell surface expression of nucleolin is not a specific poperty of CEM cells, since all the cells which were studied expresssed at different degrees cell surface P95/nucleolin; the level of detection being lower in murine compared to human cells. However, this latter might be the consequence of the lower reactivity of the antibody that was raised against human nucleolin. It is of interest to note that murine nucleolin migrated slightly faster than the human nucleolin, this probably reflects a slight difference in its molecular weight. It should also be noted that in some samples there were partial degradation products of nucleolin, as p60 and p50 (1 +L3A).

The RD cell line has been reported not to express cell surface nucleolin (Raab de Verdugo et al., 1995), however, by our technique we could demonstrate that these cells do express cell surface nucleolin. This discrepancy could be due to slight differences in the culturing conditions of cells and/or differences in the experimental approach to detect the cell surface expressed nucleolin.

### Example 14

Expression of Nucleolin, PHAP II and PHAP I in Different Types of Human and Murine Cells

In order to show expression of nucleolin, PHAP II and PHAP I in different types of human and murine cells, extracts were first purified on the affinity column containing 5[Kψ(CH₂N)PR]-TASP in order to recover the V3 loop-BPs: nucleolin, PHAP II and PHAP I (as described in the legend of FIG. 9 +L). The purified proteins were then eluted by 2-fold electrophoresis sample buffer and analyzed by immunoblotting using rabbit antiserum against the NH₂-terminal of each of nucleolin, PHAP II and PHAP I (1 +L3B). Both human and murine cells were found to express nucleolin, PHAP II and PHAP I at various levels. In some cells, PHAP II was resolved as a doublet which might account for a post-translational modification of this protein in these cells. The level of PHAP I in murine cells was low under our experimental conditions where the antiserum was raised against the human PHAP I peptide. it might therefore be possible that our antiserum reacts poorly with the murine homologue. The rabbit antiserum against the PHAP I peptide identified a 20 kDa protein in different types of human cells (1 +L3B). This latter could represent a partially degraded product of PHAP I.

### Example 15

The Purified Preparation of the V3 Loop-BPs Inhibit HIV-1 Infection

The purified preparation of the V3 loop-BPs contained P95/nucleolin, P40/PHAP II and P301PHAP I, and also the degradation product of nucleolin p60 (as shown in FIG. 9 +L). When assayed on a single cycle HIV infection, the purified preparation of the V3 loop-BPs inhibited in a dose dependent manner virus infection, with more than 80% inhibition observed at 10 μg/ml of the V3 loop-BPs (1 +L4A). The capacity of the purified V3 loop-BPs to inhibit HIV infection, suggested their potential interaction with HIV particles, and provided a mechanism by which the purified proteins could block what might be happened under normal conditions, i.e., the interaction of HIV particles with the cell surface expressed V3 loop-BPs.

The purified preparation of the V3 loop-BPs was also tested in a conventional infection, i.e., multiple cycles of infection (1 +L4B). For this purpose, the HIV-1 Lai stock was first incubated at 4° C. in the presence of different concentration of the V3 loop-BPs before the addition of cells and further incubation at 37° C. HIV production measured at 5 days p.i. indicated that there was a significant level of inhibition of virus production in a dose dependent manner. At 10 μg/ml of the purified preparation of the V3 loop-BPs, the degree of inhibition of virus infection was 91% (1 +L3B).

### Example 16

The Effect on HIV Infection of Rabbit Antisera Raised Against Peptides Corresponding to Nucleolin, PHAP II and PAP I

Rabbits immunized against synthetic peptides corresponding to the NH₂-terminal and internal sequences of nucleolin, PHAP II and PHAP I produced high titred antibodies against their respective peptide antigen. However, all the antibodies did not react with the native proteins present in the purified preparation of the V3 loop-BPs (Table 4). For example, although a similar reactivity with the respective peptide was observed for serum against the NH₂-terminal and internal peptides of nucleolin, the serum against the internal peptide reacted very poorly, if any, with the purified preparation of the V3 loop-BPs. Similarly, serum against the internal sequence of PHAP II manifested a very low reactivity with the V3 loop-BPs. In contrast, serum against the internal sequence of PHAP I reacted significantly, albeit less than that against the NH₂-terminal end, with the V3 loop-BPs (Table 4). The reactivity of each serum with the respecive peptide was highly specific. Table 5 gives the results obtained with sera against the NH₂-termini of nucleolin/PHAP II/PHAP I, along with a serum against the NH₂-terminal sequence of CXCR4 and a control serum against an internal sequence of the U₁ small nuclear ribonucleoprotein (RNP) C. Each serum reacted only with the respective peptide in an ELISA or with the respective protein in an Immunoblotting assay (Table 5 and 1 +L0). Among such antibodies, only serum against either nucleolin, PHAP II, PHAP I reacted with the preparation of the V3 loop-BPs, whereas serum against CXCR4 did not react at all (Table 5). This latter provides further evidence to indicate that the purified preparation of the V3 loop-BPs does not contain CXCR4. Consistent with this, serum raised against the preparation of the V3 loop-BPs did not react with the CXCR4peptide (Table 5) or with the 50 kDa protein (corresponding to CXCR4) in the crude cell extracts (not shown). Rabbit antiserum raised against RNP U₁C peptide reacted only with the respective peptide, thus emphasizing the specific nature of the antibodies used in our experiments (Table 5).

Rabbit antisera raised against NH₂-termini of nucleolin/PHAP II/PHAP I resulted in a dilution-dependent inhibition of HIV infection (1 +L5). The pre-immune sera had no effect on HIV infection at 1:200 fold dilution (not shown). In general, sera from immunized rabbits with different unrelated antigens, manifested an undefined activity against HIV infection when used at dilutions less than 200 fold. For this reason, routinely as controls in individual experiments, we included sera or purified immunoglobulins of rabbits immunized with unrelated peptides. For example, FIG. 8 shows that serum from a rabbit immunized with a synthetic peptide corresponding to RNP U₁C does not have a significant effect on HIV infetion. The anti-HIV effect of any rabbit antiserum raised against synthetic peptides corresponding to nucleolin/PHAP II/PHAP I was highly correlated to its reactivity with the corresponding native protein present in the purified preparation of the V3-loop BPs. Indeed, sera which manifested a significant reactivity with the V3 loop-BPs preparation exerted a strong inhibitory effect on HIV infection. On the other hand, sera reacting poorly with the corresponding native protein exerted a slight inhibitory effect on HIV infection (Table 5).

### Example 17

Peptide Affinity Purified Antibodies Against Either Nucleolin, PHAP II, or PHAP I Inhibit HIV Infection

The inhibitory effect of rabbit antisera against nucleolin/PHAP II/PHAP I on HIV infection was further confirmed by using immunoglobulin fractions purified by affinity columns containing the respective synthetic peptides as described in the “Experimental Procedures”. Such peptide affinity purified immunoglobulins at 100 μg/ml resulted almost complete inhibition of HIV infection, as it was observed by the dramatic reduction of HIV production at 5 days p.i. (1 +L6A). Virus production increased slightly in samples treated with anti-nucleolin and anti-PHAP II at 6 days p.i. (1 +L6B). This latter was most probably due to subsequent cycles of infection by the virus amplified at early phases of infection, since antibodies were added only at the time of infection and at day 3 post-infection. The control IgG fraction (at 100 μg/ml) from a rabbit immunized against RNP U₁C did not have any effect on HIV infection (FIGS. 16A and B +L). The fact that peptide affinity purified antibodies against either one of the V3 loop BPs were capable of inhibiting HIV infection, indicated that nucleolin, PHAP II and PHAP I are expressed or accessible on the cell surface, and that these three proteins are probably associated in the same complex, which hereafter will be referred to as V3 loop binding proteins complex (V3 loop-BPs complex). Consequently, although previous results indicated that individual proteins in the V3 loop-BPs complex can bind the V3 loop (FIGS. 9 and 10 +L), the binding of antibodies to any one of the three proteins may lead to conformational changes in the complex and thus block its function.

At 5 days p.i., more than 85% inhibition of HIV production was observed by individual antibodies against nucleolin, PHAP II, or PHAP I, each at 100 mg/ml. In another experiment, the IC₅₀ values on the 5th day of infection by peptide affinity purified antibodies against nucleolin, PHAP II, and PHAP were assayed to be 25, 8, and 10 mg/ml, respectively. In this respect, it is of interest to note that in a recent paper, rabbit antibodies against CXCR4 had to be used at 0.5 to 1 mg/ml in order to obtain 80% inhibition of HIV infection (Feng et al., 1996).

### Example 18

Antibodies Raised Against Either Nucleolin, PHAP II, or PHAP I Inhibit the Binding of HIV Particles to Cells

In order to understand the mechanism by which antibodies against either nucleolin, PHAP II, or PHAP I inhibit HIV infection, the effect of these antibodies was assayed on the binding of virus particles to cells, by monitoring the concentration of the major viral core protein p24 (Krust et al., 1993). A monoclonal antibody (mAb CB-T4) against the gp120 binding site in the CD4 molecule was used in order to determine the nonspecific binding of HIV to the the cell surface. These binding experiments were carried out at 37° C. rather than at 4° C., in order to respect the physiological conditions of virus infection. Consequently, the amount of virus bound (or associated) to cells represents the sum of intracellular virus and extracellular virus which is bound on the cell surface. In the presence of 5 μg/ml of mAb anti-CD4, HIV-1 entry and infection was inhibited by more than 90% (not shown), whereas the binding of HIV was affected only by 62%, thus indicating that about 38% of HIV particles become associated with the cell surface in a nonspecific manner compared to 62% which represents functional binding (1 +L7). Interestingly, 100 μg/ml of each of peptide affinity purified antibodies against nucleolin, PHAP II and PHAP I inhibited HIV binding by 65 to 75%, i.e., at least at a similar extent or much more compared to the effect of the anti-CD4 mAb (1 +L7). Treatment of cells with both mAb anti-CD4 and any one of the antibodies against nucleolin, PHAP II and PHAP I, did not increase the inhibition of binding occuring with the mAb anti-CD4 alone (1 +L7). Thus, the residual 25 to 38% binding observed in the presence of either antibodies against nucleolin, PHAP II and PHAP I should also represent nonspecific binding of HIV to cell surface components, such as heparan-sulfates as it has been reported previously (Patel et al., 1993). Whatever is the case, such nonspecific binding is not functional as it was demonstrated by the action of mAb anti-CD4 and the different antibodies against the V3 loop-BPs (1 +L4). These results are consistent with our previous data showing that the 5[Kψ(CH₂N)PR]-TASP pseudopeptide of HIV entry inhibits HIV binding in a similar manner as the different antibodies against nucleolin, PHAP II and PHAP I (Callebaut et al., 1997b).

### Example 19

High Affinity Binding of gp120 to the V3 Loop-BPs

In an ELISA type experiment HIV-1 gp120 corresponding to that of the HIV-1 Lai isolate, was shown to bind in a dose-dependent manner the purified V3 loop-BPs (1 +L8). No binding was observed between HIV-1 gp41 or histone H3 with the V3 loop-BPs (1 +L8). It could be argued that the binding of gp120 to the V3 loop BPs is simply the consequence of a nonspecific interaction between basic amino acid residues in the gp120 and the acidic domains in nucleolin, PHAP II and PHAP I. However, this is most unlikely, since histone H3B which is rich in basic amino acids does not at all bind the V3 loop BPs. It is most unlikely that the high affinity binding of gp120 to the V3-BPs (Table 9) is simply the consequence of unspecific interaction between basic amino acid residues in gp120 and the acidic domains P95, P40 and P30, since histone H3 which is rich in basic aminoacids does not bind at all (data not shown). This latter and the observation that binding does not occur with gp41, confirm that the binding of gp120 to the V3 loop-BPs is specific. In these experiments, as a control mAb, we used mAb OKT4A specific for CD4. No significant reactivity was detectable between mAb OKT4A and the V3 loop-BPs. Similarly, no significant reactivity was observed with the rabbit anti-CXCR4 antibodies (see the legend of 1 +L8). Consistent with the results of immunoblot analysis shown in 1 +L0, the preparation of the V3 loop-BPs was therefore not contaminated with the CD4 receptor or the chemokine receptor CXCR4.

With such a preparation of the V3 loop BPs, we next determined the kinetic rate and equilibrium affinity constants of 5[Kψ(CH₂N)PR]-TASP and its non-reduced counterpart 5[KPR]-TASP, along three different preparations of gp120 corresponding to lymphotropic HIV-1 isolates Lai, MN, and SF2. In addition, an unglycosylated form of gp120 corresponding to HIV-1 SF2 was used. As shown in Table 6, the pseudopeptide inhibitors of HIV entry manifested a high affinity binding to the V3 loop-BPs, with the equilibrium affinity constants Kₐ values of 9.6×10⁹ M⁻¹ and 1.5×10⁸ M⁻¹ for 5[Kψ(CH₂N)PR]-TASP and 5[KPR]-TASP, respectively. The higher Kₐ value observed for 5[Kψ(CH₂N)PR]-TASP is in accord with its higher activity on HIV entry (Callebaut et al., 1996). The gp120 from the three different HIV-1 isolates also manifested a high affinity binding to the V3 loop-BPs, with Kₐ values of 2.1×10⁸ M⁻¹, 4.3×10⁸ M⁻¹, and 2.3×10⁹ M⁻¹ for gp120 of Lai, MN, and SF2, respectively (Table 6). Interestingly, although the unglycosylated form of gp120/SF2 manifested about 10-fold reduction compared to the glycosylated counterpart, its affinity was still high with a Kₐ value of 1.6×10⁸ M⁻¹ (Table 6). This latter suggests that the polysaccharide side chains of the native gp120 molecule probably are not necessary for its binding to the V3 loop-BPs.

Under similar experimental conditions, the Kₐ values of gp120-Lai, gp120-MN and gp120-SF2 for soluble CD4 were 10.8, 7.7 and 2.1×10⁸ M⁻¹, respectively (Table 9), in accord with previously published values (Lasky et al., 1987). Therefore, the affinity of gp120 to bind CD4 and the V3-BPs was of the same order.

Finally, two synthetic V3 loop peptides, corresponding to the amino acid sequence of the T-cell tropic HIV-1 Lai and of the macrophage-tropic HIV-1 Ba—L isolate (Materials and Methods), were investigated for their capacity to interact with the purified V3-BPs. Both of these V3 loop peptides were found to bind V3-BPs, however, in contrast to gp120 and to 5[Kψ(CH₂N)PR]-TASP, they manifested somewhat lower affinity of binding. The Kₐ value of V3 loop-Lai and V3 loop-Ba-L to bind the V3-BPs was 5.1×10⁶ M⁻¹ and 1.5×10⁶ M⁻¹, respectively. By extrapolation therefore, the affinity of the V3 loop to bind the V3-BPs might be at least three-fold higher for the T-cell tropic compared to the macrophage-tropic HIV-1 isolates. The presence of a high number of basic residues in the V3 loopLai compared to the V3 loop-Ba-L could account for this difference (Callebaut et al., 1996). It is of interest to note that the lower affinity of the V3 loop Ba-L to bind the V3-BPs compared to that of the V3 loop-Lai, corresponds well with the lower inhibitory activity of the antibodies against nucleolin, PHAP II, and PHAP I on HIV-1 Ba-L compared to HIV-1 Lai infection (FIGS. 24 and 25 +L).

In order to demonstrate whether 5[Kψ(CH₂N)PR]-TASP and gp120 interact with the same domains in the V3 loop-BPs, the binding of 5[Kψ(CH₂N)PR]-TASP and gp120 to the V3 loop-BPs was investigated in the presence of increasing concentrations of gp120 and 5[Kψ(CH₂N)PR]-TASP, respectively. The results illustrated that gp120 blocks the binding of 5[Kψ(CH₂N)PR]-TASP to the V3 loop-BPs, and alongwise 5[Kψ(CH₂N)PR]-TASP has the capacity to block the binding of gp120 to the V3 loop-BPs (FIG. 11 +L, Panels A and B). The existence of such a competetion between 5[Kψ(CH₂N)PR]-TASP and gp120 to bind the V3 loop-BPs, indicates that the inhibition of HIV entry exerted by 5[Kψ(CH₂N)PR]-TASP is the consequence of inhibition of gp120 binding to the V3 loop-BPs. In accord with this, there is a close correlation between the anti-HIV activity in cell cultures and the capacity to block the binding of gp120 to the V3 loop-BPs, of 5[KψW(CH₂N)PR]-TASP and its non-reduced counterpart 5[KPR]-TASP. Indeed, the IC₅₀ value for the inhibition of HIV infection and gp120 binding to the V3 loop-BPs is 0.5 μM and 25 nM, respectively, for 5[Kψ(CH₂N)PR]-TASP, whereas that for 5[KPR]-TASP is 5 μM and 300 nM, respectively.

### Example 20

HIV-1 gp120 Binds to the V3 Loop-BPs Via the V3 Loop Domain

The results obtained in different experiments suggested that gp120 could interact with the V3 loop-BPs through its V3 loop domain. In accord with this, the binding of gp120 to the V3 loop-BPs was inhibited in the presence of mAb N11-20 directed against the V3 loop (1 +L9C). A number of monoclonal antibodies, directed against different domains in the gp120 molecule, were then used to further characterize the binding domain in gp120 (Table 7). Two mAbs mAbs B12 and ADP39 directed against the CD4 binding site in gp120 had no effect. Similarly, mAbs AD3 and 110-1 directed against NH₂- and COOH-teminus of gp120, respectively, did not have any apparent effect on the binding of gp120 to the V3 loop-BPs. On the other hand, 3 monoclonal antibodies (mAbs N11-20, 110-4, and V3-21) directed against the V3 loop inhibited the binding at IC₅₀ values around 100 nM. The mAb 110-D against an epitope situated about 50 amino acids down-stream of the COOH-terminus of the V3 loop had no effect, whereas mAb 110C against an epitope about 16 amino acids upstream of the NH₂-terminus of the V3 loop resulted in a significant inhibition of binding, similar to that observed with anti-V3 loop mAbs (Table 7). The epitope of mAb 110C being close to the NH₂-terminus of the V3 loop might cause conformational changes in the V3 loop domain of gp120, leading to the inhibition of gp120 binding to the V3 loop-BPs. These results point out that the V3 loop domain is the binding site in gp120 for the V3 loop-BPs; a conclusion which is consistent with the previous results showing that the V3 loop-BPs (P95/nucleolin, P40/PHAP II and P30/PHAP I) bind the synthetic V3 loop peptide (FIGS. 9 and 10 +L), gp120 binds the V3 loop-BPs with a high affinity, and the pseudopeptide-inhibitor of HIV entry 5[Kψ(CH₂N)PR]-TASP blocks the binding of gp120 to the V3 loop-BPs.

### Example 21

Antibodies Against Either Nucleolin, PHAP II, or PHAP I, Inhibit the Binding of gp120 to the V3 Loop BPs

The binding of gp120 to the V3 loop-BPs was inhibited by more than 75% in the presence of 1:250th dilution of either serum against nucleolin, PHAP II and PHAP I. At the same dilution, rabbit antiserum against the purified preparation of the V3 loop BPs inhibited by 77%, whereas control serum against histone H2B had no effect (Table 8). Mixing different sera together did not result in a significant increase in the inhibition of gp120 binding to the V3 loop-BPs, compared when each serum was used alone (not shown). The 50% inhibition of gp120 binding to the V3 loop-BPs was observed in the presence of the effective sera at dilutions ranging from 1:500 to 1:1000 (Table 8). The observation that serum against either nucleolin, PHAP II and PHAP I, was capable of inhibiting the binding of gp120 to the V3 loop-BPs indicated once again that these three proteins should exist together in the same complex. Indeed, if the gp120 was binding independently to nucleolin, PHAP II and PHAP I, then antibodies against one of the three should not have resulted in a significant inhibition of binding. Similar experiments were carried out using peptide affinity purified polyclonal antibodies; at 10 μg/ml of antibody against either nucleolin, PHAP II, or PHAP I, the binding of gp120 was inhibited by more than 70%.

### Example 22

Recovery of Nucleolin/PHAP II/PHAP I from the Surface of Activated Peripheral Blood Mononuclear Cells

The results presented above indicated that nucleolin, PHAP II and PHAP I should be associated in the same functional complex and probably be expressed on the cell surface. Using the biotin-labeled 5[Kψ(CH₂N)PR]-TASP pseudopeptide and intact CEM cells, we could the recovery of cell surface expressed nucleolin but not that of PHAP II and PHAP I (FIG. 8 +L). This might have been due to different affinities of 5[Kψ(CH₂N)PR]-TASP to bind each component of the V3 loop-BPs, and/or the stability of the complex formed between each component and the 5[Kψ(CH₂N)PR]-TASP pseudopeptide. In contrast to the CEM cell line, cell surface expressed nucleolin/PHAP II/PHAP I could be recovered by using peripheral blood lymphocyes. Indeed, following incubation of intact blood lymphocytes with the biotin labeled 5[Kψ(CH₂N)PR]-TASP, we could recover cell surface expressed nucleolin/PHAP II/PHAP I by complexing to the pseudopeptide (2 +L0). In view of these observations, it is possible to suggest that the recovery of the cell-surface expressed nucleolin/PHAP II/PHAP I might be cell type specific.

Subcellular Localisation of nucleolin/PHAP II/PHAP I

The observation that purified antibodies directed against peptides corresponding to nucleolin, PHAP II and PHAP I inhibit HIV infection, suggested that these proteins are expressed on the cell surface. In FACS analysis however, these same antibodies were found not to be suitable for the detection of the cell surface expressed nucleolin/PHAP II/PHAP I (not shown). This latter was probably due to the small proportion of antibodies specific for nucleolin/PHAP II/PHAP I present in the pool of the polyclonal antibodies in the rabbit antisera. A similar observation has also been reported by Feng et al. (1996), in which rabbit polyclonal antibodies against a synthetic peptide corresponding to the CXCR4 cofactor inhibited HIV infection, but in FACS analysis did not detect the cell surface expression of CXCR4. On intact peripheral blood lymphocytes, the biotin-labeled 5[Kψ(CH₂N)PR]-TASP was able to form a stable complex with the nucleolin/PHAP II/PHAP I and the complex was recovered using avidin agarose, thus providing further evidence to support the suggestion that the V3 loop-BPs are expressed on the cell surface. Comparison of the estimated amount of the nucleolin/PHAP II/PHAP I found in crude cytoplasmic extracts with that isolated from the cell surface, suggested that cell surface expressed V3 loop-BPs could represent less than 20% of that found in the cytoplamic fraction. It should be emphasized that nucleolin/PHAP II/PHAP I do not contain hydrophobic domains. Therefore, the fate of these proteins should be dependent on their interaction with other protein partner(s) which are responsible for their transport and cell surface expression. Cell lines, such as CEM, MOLT4, and Jurkat, appear to express higher levels of nucleolin compared to peripheral blood lymphocytes. Consequently, the biotin-labeled 5[Kψ(CH₂N)PR]-TASP might recover mostly nucleolin from the surface of such cells (as shown for CEM cells in FIG. 8 +L). It might also be possible that recovery of cell surface nucleolin/PHAP II/PHAP I by complexing with the 5[Kψ(CH₂N)PR]-TASP pseudopeptide is variable according to the cell line studied. Whatever is the case, the recovery of nucleolin/PHAP II/PHAP I from the surface of blood lymphocytes supports the suggestion that these proteins are indeed expressed on the cell surface. Preliminary results have suggested that the 5[Kψ(CH₂N)PR]-TASP pseudopeptide manifests higher affinity towards nucleolin compared to PHAP I and PHAP II. Accordingly, it might be possible that complexes formed between 5[Kψ(CH₂N)PR]-TASP and cell surface nucleolin are more stable compared to that formed with PHAP II and PHAP I. and therefore are recovered more efficiently. cl Example 23

Inhibition of HIV Infection by Rabbit Antisera Raised Against Nucleolin, PHAP II and PHAP I Peptides is Specific to the HIV Envelope Glycoproteins

To demonstrate the specificity of the antibodies directed against V3-BPs in respect to the HIV-envelope glycoprotein mediated infection of CD4⁺ cells, we investigated the infection of CEM cells with an HIV-1 pseudotyped virus harboring amphotropic MO-MLV envelope proteins. Infection of CEM cells by this pseudotyped HIV was inhibited by AZT but not by the anti-CD4 mAb CB-T4 (2 +L3), as the entry in this case is mediated by the Mo-MLV envelope proteins (Battini et al., 1996)). The polygonal antibodies raised against nucleolin, PHAP II, and PHAP I had no significant effect on the infection with this pseudotyped virus, compared to the infection in the presence of antibodies against the control cell-surface proteins (2 +L3). These results therefore indicate that the action of antibodies against nucleolin, PHAP II, and PHAP I, is specific to the infection mediated by the HIV envelope glycoproteins. In accord with this, 5[Kψ(CH₂N)PR]-TASP which inhibited HIV-1 Lai infection in CEM cells by more than 95% (2 +L4), exerted no significant effect on the infection of CEM cells by the pseudotyped HIV-1 virus (2 +L3).

The purified antibodies against either nucleolin, PHAP II and PHAP I inhibited the infection of PBMC with macrophage-tropic HIV-1 Ba-L and Ada isolates (2 +L5A), or a syncytium-inducing (92UG029A) and a non-syncytium-inducing (92BR025C) primary HIV-1 isolate (FIGS. 25B and C +L). Although, the degree of the inhibition of macrophage-tropic HIV-1 isolates by the polyclonal antibodies against nucleolin and PHAP I was not very high, such inhibition by both of these antibodies was significant since it was=50%. These results suggest that the V3-BPs are implicated in the mechanism of infection of PBMC by different HIV-1 isolates which could be distinguished by their cellular tropism. Consistent with these results, 5[Kψ(CH₂N)PR]-TASP has the capacity to inhibit infection of PBMC with these macrophage-tropic (not shown) and primary HIV-1 isolates (Callebaut et al., 1996).

### Example 24

Specific Inhibition of T Lymphocyte- and Macrophage-tropic HIV Entry by a V3 Loop Mimicking Pseudopeptide that Binds Cell-Surface Nucleolin

The 5[Kψ(CH₂N)PR]-TASP inhibitor was used here to assay its inhibitory effect on different types of HIV isolates in HeLa C4⁺ cells and in peripheral blood mononuclear cells (PBMC). The 5[Kψ(CH₂N)PR]-TASP molecule was synthesized as before but the proline residue was dehydroxyproline. This dehydroxyproline containing 5[Kψ(CH₂N)PR]-TASP was found to be 5 to 10 fold more active than the previously synthesized molecule with proline (this latter construct when used, will be referred with an asterix 5[Kψ(CH₂N)PR]-TASP*). The 5[QPQ]-TASP construct was used as a control peptide which has no effect on HIV infection.

Two types of HeLa cells expressing the bacterial lacZ gene under the control of the HIV-1 LTR were used (provided by O. Schwartz and P. Charneau, Institut Pasteur):

HeLa P4: expressing recombinant CD4

HeLa P4-C5 expressing recombinant CD4 and CCR5

HIV entry and replication, results in the activation of the HIV LTR, leading to the expression of the lacZ gene. Therefore, at 24 and 48 hours post-infection, the β-galactosidase activity could be measured in cell extracts directly. Consequently, this latter technique could be used to monitor HIV entry into cells. The HIV dose used corresponded to the amount of virus containing 20-40 ng/ml of p24.

When applicable, we used AZT (5 μM) to inhibit the activity of the viral reverse transcriptase, thus resulting in more than 99% inhibition of HIV infection monitored by the production of p24. The value of b-galactosidase activity obtained in the presence of 5 μM AZT was referred to as the background value in each experiment We also used the anti-CD4 mAb CBT4 which is directed against the gp120 binding site in the CD4 molecule. This antibody blocks the CD4-dependent binding of HIV particles to cells, and thus leads to almost complete inhibition of HIV infection (Valenzuela et al., 1997).

1. The Potent Anti-HIV Effect of 5[Kψ(CH₂N)PR]-TASP in HeLa Cells

a) The Inhibition of HIV Entry into HeLa Cells by 5[Kψ(CH₂N)PR]-TASP is Specific to the HIV Envelope Glycoproteins

HeLa cells (P4 and P4-C5) were treated (incubation at 37° C., 30 min) with AZT (5 μM), a-CD4 mAb CBT4 (5 μg/ml), the control peptide 5[QPQ]-TASP (5 μM) and 5[Kψ(CH₂N)PR]-TASP (at 0.05 to 5 μM) before infection with the different HIV isolates. The β-galactosidase activity was assayed at 48 hours post-infection as a measure of HIV entry (2 +L8).

The entry of HIV-1 LAI (T lymphocyte-tropic) is inhibited by 5[Kψ(CH₂N)PR]-TASP in both HeLa P4 and HeLa P4-C 5 cells. The degree of inhibition is dose dependent and is comparable in both types of cells.

5[Kψ(CH₂N)PR]-TASP is also effective on the entry of HIV-1 Ba-L (Macrophage (M)-tropic) and HIV-2 ROD.

There is only a slight effect on SIVmac entry, but this effect is not dose dependent. Thus suggesting that the slight inhibitory effect on SIV entry is not a specific effect of 5[Kψ(CH₂N)PR]-TASP.

HIV-1 pseudotyped with VSV envelope glycoprotein (VSV-HIV-1) entry is not affected by the mAb α-CD4 but virus replication is inhibited by AZT. The 5[Kψ(CH₂N)PR]-TASP molecule even at high concentrations had no effect on the VSV-HIV pseudotyped virus entry and replication, thus confirming that the inhibitory effect of 5[Kψ(CH₂N)PR]-TASP is specific to virus particles presenting the HIV envelope glycoproteins.

b) Inhibition of HIV Entry by 5[Kψ(CH₂N)PR]-TASP in HeLa Cells Infected by Different Viral Isolates. The 50% Inhibitory Concentration (IC₅₀) of the Inhibitor is Given

The results are presented in Table 11.

The viral isolates were:

T lymphocyte tropic: HIV-1 LAI, HIV-2 ROD*

Macrophage tropic: HIV-1 Ba-L, HIV-1 JRCSF

Dual tropic (i.e. both T and M tropic): HIV-1 89.6, HIV-2 CBL

Primary HIV-1 isolate SI: HIV-1 UGO29A

HIV-1 pseudotyped with VSV envelope glycoprotein (VSV-HIV-1). HeLa P4-C5 cells were used to infect by HIV-2 ROD since infection of HeLa P4 cells were less efficient by this viral preparation.

c) 5[Kψ(CH₂N)PR]-TASP Inhibits Entry of HIV-1 Isolates Resistant to Antiviral Drugs

The results are presented in 2 +L9.

The HIV-1 isolates were:

HIV-1 Ba-L

HIV-1 AZT resistant (provided by Dr. F. Brun Vezinet)

HIV-1 Saquinavir (protease inhibitor) resistant*

HIV-1 Nevirapine (nonnucleoside reverse transcriptase inhibitor) resistant* * Provided by the NIH AIDS Research and Reference Reagent Program

In these experiments, the background values of the β-galactosidase activity were assesed by the samples incubated with the a-CD4 mAb. Furthermore, the effect of heparin (5 mg/ml) was investigated. Heparin had no significant effect on the HIV-1 Ba-L but the three other virus isolates were drastically inhibited.

2. The Anti-HIV Effect of 5[Kψ(CH₂N)PR]-TASP in PBMC Cultures

a) 5[Kψ(CH₂N)PR]-TASP Inhibits Entry of Different HIV-1 Isolates in PBMC

PBMC treated with a-CD4 (5 μg/ml), the control peptide 5[QPQ]-TASP (5 μM), 5[Kψ(CH₂N)PR]-TASP (in μM concentrations as indicated) were infected with the different viral isolates. Virus production was monitored by the concentration of p24 in the culture medium at 6 days post-infection (3 +L0). The HIV-1 isolates were:

HIV-1 ELI (African isolate which infects preferentially PBMC)

HIV-1 Ba-L*, SF162*, Ada-M* (monocyte/macrophage tropic isolates)

HIV-1 UGO29A* (Primary SI isolate)

HIV-1 Retrovir (AZT) resistant* (this is also a primary isolate) * Provided by the NIH AIDS Research and Reference Reagent Program

5[Kψ(CH₂N)PR]-TASP was active against HIV-1 ELI, Ba-L, SF162, UGO29A, and Retrovir resistant isolate, with IC₅₀ values ranging from less than 0.05 to 0.5 μM. Thus indicating that 5[Kψ(CH₂N)PR]-TASP is effective against different types of HIV-1 isolates in PBMC cultures. On the other hand, 5[Kψ(CH₂N)PR]-TASP had no effect on HIV-1 Ada-M isolate, and even, there was a dose dependent enhancement of virus infection. This could be an intrinsic propriety of the HIV-1 Ada isolate as a consequence of its repeated passage. It is of interest here to note, that previously synthetic V3 loop peptides have been reported to either inhibit or enhance HIV-1 infection. However, the mechanism of this latter remains still unknown. Whatever is the case, the inhibition of virus production in PBMC infected by HIV-1 UGO29A and Retrovir resistant isolates, points out the capacity of 5[Kψ(CH₂N)PR]-TASP to inhibit efficiently primary HIV isolates.

3. The Inhibitory Effect of 5[Kψ(CH₂N)PR]-TASP in Comparison with that of Chemokines, RANTES, MIP-1α and MIP-1β

a). Chemokines Inhibit Poorly HIV Infection in HeLa Cells

The effect of chemokines, SDF-1, RANTES, MIP-1α and MIP-1β (in nM concentrations as indicated in 3 +L1) was investigated against HIV-1 LAI infection of HeLa P4 cells and against HIV-1 Ba-L infection of HeLa P4-C5 cells. The β-galactosidase activity was used at 48 hours post-infection in order to monitor viral entry. AZT was at 5 μM, mAb CBT4 (a-CD4) was at 5 μg/ml, mAb specific for the V3 loop of HIV-1 LAI (a-V3)* was at 5 μg/ml, and 5[Kψ(CH₂N)PR]-TASP was at 5 μM.

* As it was expected, mAb a-V3 blocked completely HIV-1 LAI entry, whereas it had no effect on the HIV-1 Ba—L.

b). Association of Chemokines and 5[Kψ(CH₂N)PR]-TASP Results in a Synergist Effect on HIV Infection in PBMC

PBMC treated with AZT (5 μM), α-CD4 (5 μg/ml), 5[Kψ(CH₂N)PR]-TASP (as indicated in 3 +L2), RANTES, MIP-1α and MIP-1β (as indicated), or association of 5 nM of each of the chemokines with 0.1 μM of 5[Kψ(CH₂N)PR]-TASP were infected with the HIV-1 Ba—L isolate. Virus production was monitored by the concentration of p24 in the culture medium at 7 days post-infection.

Note. The degree of inhibition of HIV-1 Ba—L isolate by 5[Kψ(CH₂N)PR]-TASP was less efficient than that observed in the experiment presented in 3 +L0. This difference is most probably due to individual differences between different blood donors, since the viral and pseudopeptide preparations were identical in FIGS. 30 and 32 +L.

These results (FIGS. 31 and 32 +L) show that the different chemokines are poorly or not active against T- and M-tropic HIV-1 infection in HeLa cells (3 +L1), whereas they can efficiently inhibit HIV-1 Ba—L infection in PBMC (3 +L2). On the other hand, 5[Kψ(CH₂N)PR]-TASP inhibitor is effective in both cell systems, i.e., HeLa and PBMC (FIGS. 31 and 32 +L). Interestingly in PBMC cultures, combinations of low concentrations of chemokines (5 nM) with that of 5[Kψ(CH₂N)PR]-TASP (0.1 μM) results in a synergistic effect on HIV entry (3 +L2).

The differential effect of chemokines on HIV-1 Ba—L infection in HeLa P4-C5 and PBMC cultures suggest that their action is cell type specific as it has been reported previously (Naif et al., 1998).

4. The Mechanism of Action of 5[Kψ(CH₂N)PR]-TASP

In this section, the inventors provide evidence to indicate that the anti-HIV effect of 5[Kψ(CH₂N)PR]-TASP is a consequence of its binding to the cell-surface and preventing the subsequent binding and entry of HIV virions.

a) 5[Kψ(CH₂N)PR]-TASP Inhibits HIV Entry by its Capacity to Bind the Surface of HeLa Cells

HeLa cells (P4) were treated (incubation at 37° C., 30 min) with AZT (5 μM), α-CD4 (5 μg/ml), the control peptide 5[QPQ]-TASP (5 μM) and 5[Kψ(CH₂N)PR]-TASP (at 0.1 to 10 μM) before infection with HIV-1 LAI (3 +L3). A group of cells treated with different doses of 5[Kψ(CH₂N)PR]-TASP were washed extensively with PBS before infection. The μ-galactosidase activity was measured at 48 hours post-infection as a measure of HIV entry. Note. The degree of inhibition of HIV entry by 5[Kψ(CH₂N)PR]-TASP was found to be comparable in cells without or with the PBS wash. These data support the suggestion that 5[Kψ(CH₂N)PR]-TASP binds to the cell surface and exerts its inhibitory effect on the HIV entry process.

b) 5[Kψ(CH₂N)PR]-TASP Inhibits the Binding and Entry of HIV Particles

HeLa cells (P4 or P4-C5) were treated with α-CD4, α-V3 (each at 5 μg/ml), the control peptide 5[QPQ]-TASP (5 μM) and 5[Kψ(CH₂N)PR]-TASP (at 0.1 and 1 μM) before infection with HIV-1 LAI (HeLa P4) or HIV-1 Ba—L (HeLa P4-C5) isolates (3 +L4). One hour after incubation at 37° C., cells were washed extensively with PBS and the amount of p24 associated with cells was maesured as an estimate for the amount of HIV binding. Similar cells washed with PBS, were treated with trypsin to elimate virus bound on the cell surface, before measuring the p24 concentration as an estimate for the amount of intracellular HIV (HIV entry).

The binding of HIV was inhibited only by 30 to 40% by (α-CD4, thus indicating that there is 60-70% nonspecific binding which is not functional, since under the same experimental conditions HIV entry was inhibited by more than 75%. The α-V3 mAb inhibited by more than 80% HIV-1 LAI binding in accord with our previously reported results, indicating that neutralizing α-V3 mAb affects both CD4dependent and CD4independent binding (Valenzuela et al., 1997). The α-V3 mAb had no effect on HIV-1 Ba—L since it is specific to the V3 loop of the HIV-1 LAI isolate. Interestingly, 1 μM 5[Kψ(CH₂N)PR]-TASP resulted in a significant inhibition of binding of HIV-1 LAI and Ba—L, and consequently, to more than 75% inhibition of HIV entry. At 1 μM of 5[Kψ(CH₂N)PR]-TASP, the degree of inhibition of HIV-1 LAI entry was comparable to that occuring in the presence of α-CD4 and α-V3 mAbs. Similarly, inhibition of HIV-1 Ba—L at 1 μM of 5[Kψ(CH₂N)PR]-TASP was significant, since entry in the presence of the pseudopeptide was inhibited at a similar extent as that obtained in the presence of α-CD4.

c) 5[Kψ(CH₂N)PR]-TASP Binds and Becomes Complexed with the Cell Surface Expressed Nucleolin

Under similar experimental conditions (37° C. in the culture medium) which result more than 90% inhibition of HIV-1 LAI infection in HeLa cells, 0.1-1 μM of the biotin labeled 5[Kψ(CH₂N)PR]-TASP becomes complexed with the cell-surface nucleolin (3 +L5). This binding however results in partial degradation of nucleolin. At 10 μM of 5[Kψ(CH₂N)PR]-TASP however, no nucleolin was recovered most probably due to its complete degradation (see below).

The degradation of nucleolin on the cell surface is specific since the detection of other cell surface antigens such as CD4, CD45, CXCR4, CCR5, and several cell-surface peptidases is not modified in cells treated with 5-10 μM of 5[Kψ(CH₂N)PR]-TASP (not shown). Furthermore, it should be noted that nucleolin in the cytoplasmic fraction does not appear to be affected even in the presence of high concentrations of 5[Kψ(CH₂N)PR]-TASP (3 +L5, section Cytoplasm).

d) The Binding of 5[Kψ(CH₂N)PR]-TASP to the Cell Surface Expressed Nucleolin Results in its Cleavage

Although the degree of cleavage is variable from one experiment to the other (since HeLa cell growth might be slightly modified), there is partial cleavage of nucleolin which could be observed at 60 minutes (3 +L6). At 6 hours post-addition of 5 μM of 5[Kψ(CH₂N)PR]-TASP, only a trace amount of degraded nucleolin could be detected (3 +L6).

e) On the Cleavage of Nucleolin

It should be emphasized that the degree of cleavage of nucleolin is much lower when the experiments are carried out at 4° C. (Callebaut et al;, 1997).

In the literature, nucleolin is well known to be partially degraded during cell growth and purification of cell extracts (Fang and Yeh, 1993). Previously, under our experimental conditions, the 60 kDa degradation product (p60) corresponding to the COOH-terminal portion of nucleolin was routinely observed during purification of nucleolin by the affinity matrix containing 5[Kψ(CH₂N)PR]-TASP. Thus it might be possible that p60 is a specific autoproteolytic byproduct as a consequence of p95/nucleolin binding to 5[Kψ(CH₂N)PR]-TASP or to the V3 loop. Whether p60 is implicated in the HIV entry process at a post-binding and fusion process by exercising the described shuttle function of nucleolin remains to be investigated. Interestingly, a truncated portion of nucleolin corresponding to its COOH-terminal was shown to bind Gag proteins of SIV and HIV (Bacharach et al., 1997). Consequently, it is plausible to suggest that a partially degraded product of nucleolin can assist the HIV core to be introduced properly into the cell.

### Example 25

The Anti-HIV Effect of Heparin is not Correlated with the Anti-HIV Effect of 5[Kψ(CH₂N)PR]-TASP

Here we have shown that 5[Kψ(CH₂N)PR]-TASP binds cell surface expressed nucleolin and results in its cleavage. This effect is specific since other cell surface proteins are not affected. Under such experimental conditions, the binding of HIV particles to CD4⁺ permissive cells is inhibited, consistent with our previous results demonstrating that antibodies against nucleolin inhibit also HIV binding. Furthermore, the inhibitory effect of 5[Kψ(CH₂N)PR]-TASP is specific on the HIV envelope glycoprotein-mediated entry process, since the pseudopeptide has no effect on the HIV-1 pseudotyped with the VSV envelope glycoproteins. As nucleolin contains several stretches of amino acids composed of aspartate and glutamate residues, it could be argued that its implication in the HIV entry process is due to nonspecific interactions with gp120, especially with the basic residues in the V3 loop. For this reason, we used heparin which because of its polyanionic nature has been shown to inhibit HIV binding, probably by interacting with the basic amino acids in the V3 loop.

a) The Anti-HIV Effect of Heparin is not Correlated with the Anti-HIV Effect of 5[Kψ(CH₂N)PR]-TASP.

HeLa cells (P4-C5) were treated (incubation at 37° C., 30 min) with AZT (5 μM), (α-CD4 mAb CBT4 (5 μg/ml) before infection with the different HIV isolates in order to obtain the background β-galactosidase activity compared to untreated cells (Control). During the same time, the different HIV isolates were treated (incubation at room temperature, 15 min) with 1, 5, and 10 μg/ml of heparin before addition to untreated cells. The b-galactosidase activity was assayed at 48 hours post-infection as a measure of HIV entry (3 +L7).

Heparin inhibits viral entry of HIV-1 LAI and HIV-2 ROD isolates which are also inhibited by 5[Kψ(CH₂N)PR]-TASP (see 2 +L8).

Heparin has no significant effect on HIV-1 Ba—L and JRCSF isolates, whereas 5[Kψ(CH₂N)PR]-TASP inhibits drastically entry of both of these isolates in these same HeLa cells with an IC₅₀ value of 0.3 μM (Table 11).

Heparin inhibits viral entry of HIV-1 Ada-M and SIV-mac isolates, whereas in these same cells, 5[Kψ(CH₂N)PR]-TASP has no significant effect on SIV entry (2 +L8) and it even enhances Ada-M entry (not shown, similar to the results shown in 3 +L0).

Taken together, the above results indicate that there is no correlation between the anti-HIV effect of heparing and that of 5[Kψ(CH₂N)PR]-TASP. Accordingly, HIV-1 Ada-M and SIV-mac isolates were very sensitive to the inhibitory effect of heparin, whereas HIV-1 Ba—L and JRCSF isolates were resistant (Figure Heparin). On the other hand, 5[Kψ(CH₂N)PR]-TASP inhibited viral entry of HIV-1 Ba—L and JRCSF (Table 11), but had no apparent inhibitory on HIV-1 Ada-M and STV-mac isolates (FIGS. 28 and 30 +L). Taken together, these observations point out that the potenial interaction of the V3 loop with nucleolin is not simply the consequence of the polyanionic nature of nucleolin.

### Example 26

Studies by Electron Microscopy to Demonstrate the Cell-surface Expression of Nucleolin, PHAP II, and PHAP I.

CEM cells permissive to HIV infection were used in these studies. These cells express nucleolin, PHAP II, and PHAP I. Furthermore, polyclonal antibodies against nucleolin, PHAP II, and PHAP I block the binding of HIV particles to these CD4+ cells, and thus infection.

Fixation and embedding were carried out under conventional techniques.

The antibodies used were peptide purified antibodies against nucleolin (0.5 mg/ml), PHAP II (0.25/ml), PHAP I (1 mg/ml), and against purified human nucleolin (0.6 mg/ml). The antibodies were used at 1/10 to 1/50 dilution, and were revealed with anti-rabbit IgG conjugated to gold particles.

Results

Gold particles were observed in the cytoplasm within clear vacuoles and also at the surface of the cell, in association with exocytose vesicles both inside and outside the plasma membrane.

A few gold particles were also scattered through the cells. Interestingly, gold particles were never observed in association with the rough endoplasmic reticulum.

PHAP I (p40) was not observed in the nucleus (3 +L8). In contrast, PHAP II (p30-3 +L9) and nucleolin (p95-4 +L0) were detected in the nucleus, including the nucleolus. In the case of nucleolin, the gold particles were numerous over the nucleolus.

Taken together, the data suggest that at least a part of the three proteins is packaged in intracytoplasmic vesicles which fuse with the plasma membrane and open to the extracellular space thereby releasing the proteins to the external surface of the cell. The released proteins adhere to the cell surface, possibly to be incorporated in the external matrix. The detection of these proteins inside intracytoplasmic vesicles, in vesicles opened at the cell surface and, finally, at the external surface of the cell, strongly suggest that these proteins are released by exocytosis.

### Example 27

Expression of the V3BPs (P95, P40, P30) on the Surface of Human Macrophages and Specificity of the Binding of 5[Kψ (CH₂N)PR]-TASP to the Macrophage-expressed V3BPs

1. Characterization and Phenotyping of the Cells Used

Peripheral mononuclear cells (PBMC) are isolated from the whole blood by the conventional methods (Ficoll-Paque, Pharmacia) from whole blood containers or from buffy-coats (Centre de Transfusion sanguine, Hôpital Necker). Monocytes are purified by adhesion of the surface of the culture dishes and are then cultured, under a adhered form, in the presence of autooguous lymphocytes during the first 5 days culture period, allowing them to diffrentiate into macrophages in the presence of the cytokines secreted by lymphocytes. The non-adherent lymphocytes are discarded by several washes of the cell cultures. The degree of purity of the macrophages has been determined by using specific antibodies in a cytofluorimetric analysis. The thus-obtained monocyte-derived macrophages (MDM) remain viable during a two-month culture period. As shown in FIG. 41a, the obtained MDM bear all the characteristics of cells belonging to the monocyte-macrophage cell lineage, expressing CD64, CD45Ro, CD11b and CD14 (Immunotech, France). In addition, the use of monoclonal antibodies directed against different HIV-1 coreceptors, such as CCR5, CXR4 and CCR3 (4 +L1b) has allowed the inventors to confirm the presence of said coreceptors at the surface of MDM before using these cultivated cells in the infection experiments with HIV-1.

2. Expression of the V3BPs at the Cell Surface of Human MDM

After harvesting the MDM in culture with a PBS/BSA(0.5%)/Azide(0.05%) solution, 5×10⁵ cells have been incubated in the presence of 2 μM of 5[Kψ (CH₂N)PR]-TASP labeled with FITC (fluorescein isothiocyanate) [P19*]. In some experiments, cells are first incubated with an excess amount of non labeled 5[Kψ (CH₂N)PR]-TASP before the addition of 5[Kψ (CH₂N)PR]-TASP-FITC. The FACScan cytofluorimetric analysis shows that 5[Kψ (CH₂N)PR]-TASP-FITC [P19*] binds to the macrophage cell surface; this binding is specific, since the binding of the 5[Kψ (CH₂N)PR]-TASP-FITC is displaced by an excess of 5[Kψ (CH₂N)PR]-TASP (4 +L2a). In order to show the nature of the target of 5[Kψ (CH₂N)PR]-TASP on the cell MDM surface, antibodies directed against P95, P40 and P30 have been used. These antibodies have been obtained by immunizing rabbits by synthetic peptides corresponding to the NH₂ extremity domain from P95, P40 and P30, as described in the Materials and Methods Section. The use of these rabbit antibodies allowed the inventors to confirm previous observations, showing that 5[Kψ (CH₂N)PR]-TASP binds to three membrane proteins expressed at the cell surface (FIGS. 42b, 42c ).

3. Confirmation of the Identity of the V3BPs: P95, P40 and P30

The ability of 5[Kψ (CH₂N)PR]-TASP to bind to the V3BPs and to form a stale complex with these proteins has allowed the inventors to purified P95, P40 and P30 that are expressed at the cell surface of the human MDM. The binding of 5[Kψ (CH₂N)PR]-TASP to the native proteins is specific: in Western blot analysis experiments, cell extracts from MDM (at Day-8 culture time period) have been first purified on a chromatographic substrate consisting in an avidin substrate on which has been coupled a biotynylated 5[Kψ (CH₂N)PR]-TASP using the protocol described by Callebaut et al. (1996) and Callebaut et al. (1997a); the purified proteins are loaded on a 12% polyacrylamide gel. After migration, proteins are transferred on a nitrocellulose membrane which is then incubated in the presence of the anti-P95, anti-P40 and anti-P30 antibodies. After staining, the results show that the purified proteins correspond to P95, P30 and P40 (4 +L3), when compared to the purified cell extracts feom CEM cell cultures used in this experiment as positive controls; These results confirm the expression of the V3BPs by the human macrophages.

The results of 4 +L3 show that in the macrophage cell extracts, two additional protein bands of respective Mw of 80 kDa and 60 kDa are revealed between the band corresponding to P95 and the band corresponding to P40; these two additional protein bands consist in degraded forms of P95, as it has been shown also for cell extracts from CEM cells. In Western blot analysis experiments, the protein bands corresponding to P95 and P60 are specifically recognized by the CC98 monoclonal antibody that is directed against P95, whereas a polyclonal antibody directed against the N-terminus of P95 does not recognize P60. These results suggest that P60 would consist in the C-terminus region of P95. In the course of the present experiments, it has been regularly observed that the degraded forms of P95 are found in the cell extracts from the CEM cell cultures that are not in a phase of division.

### Example 28

Role of the V3BPs (P95, P40 and P30) in the Human Macrophages Infection Mechanism by HIV-1.

1) Inhibition of in vitro HIV-1 Infection of Human Macrophages by 5[Kψ (CH₂N)PR]-TASP

Using micromolar concentrations of 5[Kψ (CH₂N)PR]-TASP in the present infection experiments has allowed the inventors to show the invlvement of the natural ligand(s) of 5[Kψ (CH₂N)PR]-TASP that are expressed at the cell surface in the mechanism of human macrophage infection by HIV-1. The results presented in 4 +L4 show that 5[Kψ (CH₂N)PR]-TASP inhibits, in a dose-dependent manner, the infection of a primary cell culture of human macrophages by two HIV-1 monotropic isolates, respectively BaL (4 +L4a) and Ada (FIG. 44b ). This inhibitory effect of 5[Kψ (CH₂N)PR]-TASP is more important with the BaL isolate than with the Ada isolate, as it is shown in 4 +L4. The difference in the inhibitory activity of 5[Kψ (CH₂N)PR]-TASP may be due to the variability of the V3 loops between the different HIV isolates, even if said variability is weak.

Moreover, the results of 4 +L4 show that the inhibition is observed when 5[Kψ (CH₂N)PR]-TASP is kept present in the culture medium, since when 5[Kψ (CH₂N)PR]-TASP is removed at Day-14 after HIV infection the viral production incerases. This latter result show that the presence of 5[Kψ (CH₂N)PR]-TASP in the culture medium is the causing effect of the observed inhibitory effect which supports the specific action of 5[Kψ (CH₂N)PR]-TASP. In the same experiment, no inhibitory effect of 5[QPQ]-TASP peptide, used as a control, is observed.

2. Inhibition of the in vitro HIV-1 Infection of Human Macrophages by Anti-P95, Anti-P40 and Anti-P30 Antibodies

The use of rabbit polygonal anti-P95, anti-P40 and anti-P30 antibodies has allowed the inventors to demonstrate that P95, P40 and P30 are involved in the mechanism of the human macrophages infection by the monotropic isolate HIV-1 BaL. The results presented in 4 +L5 show that the anti-P95, anti-P40 and anti-P30 antibodies inhibit the macrophage infection by HIV-1 BaL, in contrast to the control antiboy (C). It noteworthy that the use of a single antibody choosen among anti-P95, anti-P40 and anti-P30 is sufficient for blocking the viral infection. These results suggest that the three V3BPs act in concert in the same mechanism of HIV infection.

3. Inhibition of the in vitro HIV-1 Infection of Human Macrophages by the β Chemokines

The fact that the cell extracts from CEM cells that have been purified on a biotinylated 5[Kψ (CH₂N)PR]-TASP-avidin chromatography substrate do not contain CD4 or CXCR4 strongly suggest that 5[Kψ (CH₂N)PR]-TASP does not bind on the CD4 and CXCR4 receptors. Experiments have been performed in order to determine the respective role played by the β chemokines receptors, on one hand, and the V3BPs, on the other hand, in the mechanism of entry of the HIV in the human macrophages. For this purpose, the macrophage infection is realized in the presence of different combinations of antibodies/chemokines or 5[Kψ (CH₂N)PR]-TASP/chemokines. The experimental data confirm that Rantes, MIP-1α and MIP-1β inhibit the infection of human macrophages due to monotropic HIV isolates. The same type of experiments have been performed and cells have been incubated in the presence of different mixtures adjusted at non-inhibitory concentrations of each mixture constituent: [(Rantes/5[Kψ (CH₂N)PR]-TASP), (MIP-1α/5[Kψ (CH₂N)PR]-TASP), (MIP-1β/5[Kψ (CH₂N)PR]-TASP) or also [Rantes/anti-P40 or anti-P30), (MIP-1α/anti-P40 or anti-P30) or (MIP-1β/anti-P40 or anti-P30)], before adding the virus to cell cultures. The non-inhibitory concentrations of each constituent of the above-described mixtures have been choosen taking into account the results observed with 5[Kψ (CH₂N)PR]-TASP alone (4 +L4), the antibodies alone (4 +L5) or with the β chemokines alone (4 +L6). The results presented in 4 show that Rantes and MIP-1β inhibit the human macrophage infection by HIV-1 BaL in a dose-dependent manner. MIP-1α exhibit a weaker inhibitory effect than Rantes and MIP-1β. Furthermore, at the concentration of 50 ng/ml, none of these molecules exhibit any inhibitory effect on the human macrophage infection, neither the polyclonal rabbit antibodies at the concentration of 50 μg/ml or also 5[Kψ (CH₂N)PR]-TASP at the concentration of 0.1 μM. However, when used together, two by two within a mixture, these molecules induce a complete inhibition of the macrophage infection, as it is shown by the results presented in 4 and 4 +L7. The latter results suggest that CCR5 coreceptor and the V3BPs cooperate in a synergistic manner in the same mechanism during the course of infection of the target cells by HIV. This may be explained by the fact that the V3BPs play a role as much essential as the CD4 receptor in the binding of the viral particles on the target cells. The CCR5 and/or CXCR4 would then allow the fusion between the cell- and the viral-membrane leading to the virus entry within the cell. The V3 BPs, CCR5 and CXR4, which are structurally different from CD4, would specifically bind the viral envelope glycoproteins and would play the role of HIV coreceptors at the level of the subsequent steps following the gp120-CD4 binding and preceding the entry of HIV in the permissive cells.

### Example 29

5[Kψ (CH₂N)PR]-TASP is Useful for a Pharmaceutical Use

a) Reproducible Synthesis of 5[Kψ(CH₂N)PR]-TASP

5[Kψ(CH₂N)PR]-TASP has been synthesized, at least, at 10 different occasions, and at each time the product was active against HIV entry at similar IC₅₀ values.

b)Studies on the Solubility of 5[Kψ(CH₂N)PR]-TASP

5[Kψ(CH₂N)PR]-TASP is highly soluble in water and in PBS which contains physiological concentrations of sodium chloride (0.9% NaCl).

The solubility of 5[Kψ(CH₂N)PR]-TASP in PBS>10 mg/ml which corresponds to about 3 mM.

c) Studies on the Toxicity of 5[Kψ(CH₂N)PR]-TASP in Rats

Males rats about 100 gm (Strain Sprague Dawley; 2 rats per point) were injected intravenously through the jugular vein with different doses of 5[Kψ(CH₂N)PR]-TASP in PBS.

100 μl PBS: Controls

300 μg of 5[Kψ(CH₂N)PR]-TASP in 100 ml PBS: 3 mg/kg.

30 μg of 5[Kψ(CH₂N)PR]-TASP in 100 ml PBS: 0.3 mg/kg.

3 μg of 5[Kψ(CH₂N)PR]-TASP in 100 ml PBS: 0.03 mg/kg.

No apparent effects on the behaviour of rats were observed, even at 3 mg/kg of 5[Kψ(CH₂N)PR]-TASP. At 48 hours, the rats were sacrificed and examined. No apparent effect was observed in rats injected with 5[Kψ(CH₂N)PR]-TASP.

d) The Antigenicity of 5[Kψ(CH₂N)PR]-TASP in Rabbits

5(KPR)-TASP, the nonreduced counterpart of 5[Kψ(CH₂N)PR]-TASP, is rapidly degraded in sera from control or HIV⁺ individuals, with a half life of about 1 hour. In contrast, the half life of 5[Kψ(CH₂N)PR]-TASP under similar conditions should be very long, since less than 20% becomes inactivated after 18 hours of incubation at 37° C. (Callebaut et al., 1997).

Immunization of rabbits with 5[Kψ(CH₂N)PR]-TASP using CFA and IFA resulted in the production of antibodies specific to the pseudopeptide. However, interestingly, these antibodies did not block the anti-HIV effect of 5[Kψ(CH₂N)PR]-TASP, although they appeared to be specific to the Kψ(CH₂N)PR sequence. These observation are consistent with our previous results pointing out that the structutural requirements for the anti-HIV effect of 5[Kψ(CH₂N)PR]-TASP is mostly conformational.

In accord with the short half life of 5(KPR)-TASP in serum, this peptide when used as an antigen to immunize rabbits, it induced a very poor immune response, if any, in rabbits.

Taken together, the inventors data indicate that the rabbit antibodies were specific to 5[Kψ(CH₂N)PR]-TASP. They recognized also the non-reduced counterpart of 5[Kψ(CH₂N)PR]-TASP, i.e. 5(KPR)-TASP, but they did not react with 5(RPR)-, 5(RPK)-, or 5(KGQ)-TASP. Therefore, the antibodies did not react with the template of the TASP construct, but they reacted with the part of the 5[Kψ(CH₂N)PR]-TASP molecule which is not implicated in its anti-HIV activity. Furthermore these antibodies are specific to the motif KP.

These antibodies do not neutralize the effect of the 5[Kψ(CH₂N)PR]-TASP to bind cell-surface expressed nucleolin. Indeed, these antibodies were capable of immunoprecipitating the complex of 5[Kψ(CH₂N)PR]-TASP bound to nucleolin from the cell-surface. Therefore, the epitope recognized by these antibodies is outside the region which is responsible for binding to nucleolin.

The antibodies have the capacity to immunoprecipitate HIV-1 gp120, thus suggesting that 5[Kψ(CH₂N)PR]-TASP somehow mimics gp120, and most probably the V3 loop. This is consistent with the data that both 5[Kψ(CH₂N)PR]-TASP and the V3 loop bind similar pattern of proteins, i.e., nucleolin, PHAP II, and PHAP I.

Coordination Between Cell Surface Components Implicated in the HIV Entry Process

The inventors have demonstrated here that antibodies against any one of the V3 loop-BPs, nucleolin/PHAP II/PHAP I are as effective as the mAb anti-CD4 for the inhibition of HIV binding (1 +L7). These observations point out the existence of two specific binding events, between the gp120 molecules on the HIV-1 particles and the cell surface expressed CD4 on one hand, and nucleolin/PHAP II/PHAP I on the other hand. There should be a cooperativity between these two interactions in order to acheive functional binding, since antibodies against CD4 or antibodies against any one of the components of the nucleolin/PHAP II/PHAP I complex disrupt the functional binding process. Consequently, stable binding of HIV particles to permissive cells requires both types of interactions. The binding of soluble gp120 to the purified V3 loop-BPs in the absence of CD4 (1 +L8), might suggest that the interaction of HIV particles with nucleolin/PHAP II/PHAP I occurs independently of the interaction with CD4. However, this is probably not the case since gp120 complexed to gp41 on the surface of viral particles does not have the same conformational restrictions as the soluble gp120.

The pseudopeptide 5[Kψ(CH₂N)PR]-TASP, designed to mimic the conserved RP dipeptide motif and basic lysine and arginine residues in the V3 loop of HIV isolates, is a potent and specific inhibitor of HIV infection (Callebaut et al., 1996). Here we demonstrate that an identical pattern of proteins composed of nucleolin, PHAP II, and PHAP I, can be purified from cells using either the pseudopeptide 5[Kψ(CH₂N)PR]-TASP or a synthetic V3 loop peptide (2 +L6, lanes 3 and 4), suggesting that 5[Kψ(CH₂N)PR]-TASP can indeed mimic the V3 loop. This observation together with the fact that 5[Kψ(CH₂N)PR]-TASP is a potent inhibitor of HIV entry by binding to cell-surface components of protein in nature (Callebaut et al., 1997), suggest that nucleolin, PHAP II, and PHAP I are targets of this pseudopeptide inhibitor. The interaction of 5[Kψ(CH₂N)PR]-TASP with nucleolin, PHAP II, and PHAP I is of high affinity (Table 9). Otherwise, the purification of these V3-BPs by just a single-step would not have been possible. The control peptide 5[QPQ]-TASP construct does not bind the V3-BPs, whereas the tetravalent 4[KPR]-TASP construct, which has very little activity against HIV, binds poorly the V3-BPs and along with many other proteins. These observations therefore emphasize the unique specific nature of the pentavalent 5[Kψ(CH₂N)PR]-TASP construct. In addition, the affinity to bind the V3-BPs and the anti-HIV activity of the different TASP constructs (Callebaut et al., 1996) are tightly correlated. These three V3-BPs therefore appear to be implicated as cofactors in the process of HIV entry. Such a cofactor role of nucleolin-PHAP II-PHAP I in the HIV entry process is enforced by several observations: 1) inhibition of HIV infection using purified preparations containing nucleolin-PHAP II-PHAP I; 2) inhibition of HIV entry and infection by antibodies directed against either nucleolin, PHAP II or PHAP I peptides; 3) demonstration that gp120 binds nucleolin-PHAP II-PHAP I via its V3 loop; 4) competition between gp120 and 5[Kψ(CH₂N)PR]-TASP to bind nucleolin-PHAP II-PHAP I. By virtue to bind the V3 loop domain, nucleolin-PHAP II-PHAP I interact with the gp120 on the surface of HIV particles and thus become implicated in the HIV binding process. Consequently, agents such as the pseudopeptide 5[Kψ(CH₂N)PR]-TASP or neutralizing anti-V3 loop mAbs, block the interaction of the V3 loop domain of gp120 with cell-surface expressed nucleolin-PHAP II-PHAP I and block HIV binding and thus entry (results herein; Callebaut et al., 1997, Valenzuela et al., 1997). We found out that all human and murine cells of lymphoid or non-lymphoid origin which were investigated, express nucleolin, PHAP II and PHAP I (data not shown). In view of this, and the fact that the expression of human CD4 and CXCR4 or CCR5 is sufficient for efficient entry of different HIV-1 isolates into heterologous cells (Choe et al., 1996; Deng et al., 1996; Doranz et al., 1996; Dragic et al., 1996; Feng et al., 1996; Mebatsion et al., 1997; Schnell et al., 1997), the V3-BPs should therefore represent a complementary receptor for HIV which is not species-specific.

The mechanism by which these three V3-BPs are implicated in the process of HIV-particle binding to cells requires further investigation. It should be noted however, that besides their function in the viral particle binding process to permissive cells, nucleolin, PHAP II, and PHAP I might have a much wider implication in HIV infection. For example, nucleolin was recently reported to interact with the nuclear protein nucleophosmin or B23 (Li et al., 1996), which itself was reported to bind HIV-1 Rev and Tat proteins (Fankhauser et al., 1991; Li et al., 1997). Furthermore, B23 and HIV-1 transactivator Tat have been reported to coexist together in the nucleolus and in several other subcellular locations including the plasma membrane (Marasco et al., 1994). As nucleolin appears to have a shuttle function between the cytoplasm and the nucleus (Borer et al., 1989), it has been suggested that the association of nucleolin and B23 may represent a mechanism for nuclear localization of cellular and viral proteins (Li et al., 1996). Nucleolin, may also interact directly with HIV nucleocapsid, as a truncated portion of nucleolin corresponding to its COOH-terminal was recently reportesd to bind Gag proteins of SIV and HIV (Bacharach et al., 1997).

Previously, neutralizing antibodies specific to the V3 loop have been considered not to affect the binding of HIV particles to CD4⁺ cells (Bour et al., 1995; Moore et al., 1993). However, we and others have recently demonstrated that this conclusion was not correct and was due to the use of soluble gp120 instead of HIV particles, since although neutralizing anti-V3 loop antibodies do not affect the binding of soluble gp120 to CD4⁺ cells, they inhibit drastically the binding of HIV particles to such cells (Valenzuela et al., 1997). Interestingly, the 5[Kψ(CH₂N)PR]-TASP inhibitor of HIV entry does not affect the binding of soluble gp120 to cells, but it inhibits HIV particle binding, at a similar extent as that exerted by a neutralizing antibody specific to CD4. The degree of inhibition is not modified when 5[Kψ(CH₂N)PR]-TASP is used combined with an anti-CD4 antibody, indicating that the pseudopeptide-mediated inhibition affects specific (i.e. functional) binding of HIV particles to CD4⁺ cells (2 +L7). Consistent with this, we demonstrate here that antibodies against any one of the V3-BPs, nucleolin, PHAP II or PHAP I, are as effective as the anti-CD4 mAb for the inhibition of HIV binding (2 +L7).

Taken together, the inventor's results demonstrate the existence of two distinct domains in gp120 molecule responsible for direct binding events with the cell membrane: the first domain is the well described site of binding to CD4, whereas the second domain is the V3 loop. The CD4 binding domain is responsible for virus binding to cells, whereas the V3 loop domain with a lower binding affinity is responsible for binding to the potential nucleolin/PHAP II/PHAP I complex. This secondary binding event is as functional as the binding to CD4, since antibodies against nucleolin/PHAP II/PHAP I inhibit HIV infection. The interaction with the cofactor CXCR4 occurs probably after the binding of gp120 to CD4 and to the nucleolin/PHAP II/PHAP I complex. In accord with this, the interaction of soluble gp120 with CXCR4 has been shown to occur only after complex formation with gp120 (Lapham et al., 1996). Furthermore, SDF which is the natural ligand of CXCR4, inhibits HIV infection (Oberlin et al., 1996) without affecting the binding of HIV particles to cells (Q. J. Sattentau, personal communication). In the case of viral entry using monotropic HIV-1 isolates, it has been shown that RANTES which is a natural ligand of the cofactor CCR5, inhibits monotropic HIV-1 infection without affecting the binding of HIV particles to cells (Oravecz et al., 1996). Accordingly, it has been proposed that RANTES blocks a postbinding fusion step in the HIV entry process, and several groups have proposed that monotropic HIV-1 binding to CD4 creates a high affinity interaction site for the cofactor CCR5, and in this mechanism the V3 loop plays an important role (Wu et al., 1996, Trkola et al., 1996). However, as these latter experiments were carried out by investigating the effect of soluble gp120 on the binding of the b-chimokines MIP-1a and MIP-1b to their natural CCR5 ligand on the cell surface, it is difficult to eliminate the possibility for the existence of other complementary interactions between the gp120 and cell surface proteins in these experiments. In this respect, it is of importance to investigate the potential role of nucleolin/PHAP II/PHAP I as cofactors implicated in the binding and entry of monotropic HIV-1 isolates.

These observations point out the existence of two specific binding events occurring between gp120 molecules on the HIV-1 particles and distinct cell-surface components, namely the CD4 molecule on one hand, and nucleolin-PHAP II-PHAP I on the other hand. There should be a cooperativity between these two interactions in order to achieve functional binding, since antibodies against CD4 or antibodies against any one of the components of the V3-BPs disrupt the functional binding process. It should be noted that the interaction with the fusion-cofactor CXCR4 or CCR5 probably occurs after the binding of gp120 to CD4 and to the nucleolin-PHAP II-PHAP I. In accord with this, the interaction of soluble gp120 with CXCR4 has been shown to occur only after complex formation with CD4 (Lapham et al., 1996). Furthermore, SDF-1 which is the natural ligand of CXCR4, inhibits HIV infection (Oberlin et al., 1996) without affecting the binding of HIV particles to cells (FIGS. 27 and 33 +L). In the case of viral entry using monotropic HIV-1 isolates, it has been shown that RANTES which is a natural ligand of the cofactor CCR5, inhibits monotropic HIV-1 infection without affecting HIV binding (Oravecz et al., 1996). Accordingly, it has been proposed that RANTES blocks a post-binding fusion step in the HIV entry process. Several groups have proposed that monotropic HIV-1 binding to CD4 creates a high affinity interaction site for the cofactor CCR5, and that in this mechanism, the V3 loop plays an important role (Trkola et al., 1996; Wu et al., 1996). However, as these latter experiments were carried out by investigating the effect of soluble gp120 on the binding of the b-chemokines MIP-1a and MIP-1b to their natural CCR5 ligand on the cell surface, it remains difficult to eliminate the possibility for the existence of other complementary interactions between the gp120 and cell-surface proteins, such as the V3-BPs.

Taken together, our results indicate that there should be two distinct domains in the gp120 molecule on anr HIV particle, responsible for direct binding events with the cell membrane: the first domain is the well described site of binding to CD4 (Bour et al., 1995), whereas the second domain is the V3 loop which binds the nucleolin, PHAP II, and PHAP I (Table 10). This secondary binding event appears to be as functional as the binding to CD4, since antibodies directed against either nucleolin, PHAP II or PHAP I peptides inhibit HIV infection. Accordingly, nucleolin, PHAP II, and PHAP I represent novel targets for the development of potential anti-HIV reagents.

As it appears from the teachings of the specification, the invention is not limited in scope to one or several of the above detailed embodiments; the present invention also embraces all the alternatives that can be performed by one skilled in the same technical field, without deviating from the subject or from the scope of the instant invention.

TABLE 1
The FITC-labeled 5[KΨ(CH₂N)PR]-TASP binds to a cell-
surface protein resistant to trypsin but sensitive to proteinase K
and pronase E digestion.

| | Cell-surface expression (% positive cells) | | |
|---|---|---|---|
| Protease | TASP-Ligand | CD4 | CD26 |
| None | 100 | 100 | 100 |
| Trypsin | 92 | 24 | 100 |
| Proteinase K | 15 | 6 | 98 |
| Pronase E | 8 | 9 | 97 |

MOLT4 cells were treated with different proteases as described in “Materials and Methods” before FACS analysis using FITC-labeled 5[Kψ(CH₂N)PR]-TASP to detect the TASP-ligand, and mAbs OKT4A and Ta1 specific for CD4 and CD26, respectively. The expression of CD4 and CD26 in control cells (not treated with different proteases) was considered as 100%. Consequently the % positive cells after protease treatment were estimated by comparison with the untreated cells.

TABLE 2
The higher activity and stability of 5[KΨ(CH₂N)PR]-TASP compared to 5(KPR)-TASP.

| | 50% dose | | % activity | |
|---|---|---|---|---|
| | Effective | | Residual | |
| | Inhibition of HIV infection | Affinity to bind TASP-ligand | FCS | HIV-1⁺serum |
| TASP construct | (IC₅₀) | (EC₅₀) | 1 h/18 h | 1 h/18 h |
| 5(QPQ)-TASP | None¹ | None² | N.T. | N.T. |
| 5(KGQ)-TASP | None¹ | None² | N.T. | N.T. |
| 5(KPR)-TASP | 5 μM | 3.5 μM | 65/15 | 45/10 |
| 5[KΨ(CH₂N)PR]-TASP | 0.5 μM | 0.15 μM | 92/84 | 87/85 |
| To calculate the IC₅₀ values for the inhibition of HIV infection, different concentrations (0.25, 0.5, 1, 5, 10, 20, 50 and 100 μM) of each construct were added to CEM cells 15 min before the additon of HIV-1. The production of HIV (measured by the concentration of p24) was monitored at 4 days p.l. (7). The affinity to bind the cell-surface ligand was assayed by FACS | | | | |
| # analysis using biotin-labeled TASP constructs (as in FIG. 4). The 50% effective concentration (EC₅₀) represents the dose of the TASP-inhibitor to reveal 50% labeling of cells, considering that the maximum mean fluorescence intensity was 100%. For the stability of the TASP inhibitors, Biotin-labeled 5[KPR]- and 5[KΨ(CH₂N)PR]-TASP constructs (at 60 μM concentrations) were incubated in | | | | |
| #decomplemented serum (heated at 56° C., 30 min) from fetal calf (FCS) and from an HIV-seropositive individual (HIV-1⁺serum). After 1 and 18 hr of incubation at 37° C., aliquols were tested by FACS analysis to estimate the capacity of each construct to bind the cell-surface ligand; the results are presented as % residual activity at each time point compared to that obtained with both | | | | |
| #constructs incubated under similar experimental condition but in PBS. | | | | |

¹No effect at 100 μM;
²No binding at 20 μM;
N.T.: not tested.

TABLE 3
Homology of the amino acid sequence of the different peptides
from the V3 loop BPs to nucleolin, PHAP II and PHAP I.

| | Peptide | | |
|---|---|---|---|
| Protein | Fractions | Amino Acid Sequence | Homology (a-a) |
| A:p95 | Peak 24 | (K)QGTEIDGRSISLYYT | Nucleolin (447-563) |
| | Peak 30 | (K)GYAFIEFASFEDA(K) | Nucleolin (522-536) |
| A:p60 | Peak 24 | (K)GYAFIEFASFEDA(K) | Nucleolin (522-536) |
| | Peak 18 | (K)ALELTG | Nucleolin (361-367) |
| | | (K)QGTEID | Nucleolin (447-454) |
| | Peak 19 | (K)VTLDWAKP(K) | Nucleolin (635-644) |
| A:p40 | Peak 24 | (K)EQQEAIEHIDEVQNE | PHAP II (26-41) |
| A:p30 | Peak 27 | (K)KLELSE | PHAP I (67-73) |
| | Peak 29 | (K)KLELSENRIFGGL | PHAP I (67-80) |
| | Peak 33 | (K)SLDLFNXEVTNLNDY | PHAP I (116-131) |
| B:p95* | NH₂- | VKLAKAGKNQGDPKK | Nucleolin (1-15) |
| | terminal | | |
| A. The four proteins purified from crude cell extracts using the affinity matrix containing 5[Kψ(CH₂N)PR]-TASP (see FIG. 2), were recovered individually from the PAGE/SDS gel, digested with endo-lysine C, and the peptides were purified by and HPLC column (Experimental Procedures”). | | | |
| # Several peptides of each protein were microsequenced. The homology of the obtained amino acid (a-a) sequences to that deduced from the nucleotide sequence of cDNAs corresponding to known proteins is given. As the endolysine C cleaves peptide bonds after lysine residues, the (K) at the beginning of the | | | |
| # sequences of the different peptides and at the end of some peptides, points out that indeed in the homologous protein sequence, these peptides are adjacent to a lysine residue. The results show that p95 and p60 are homologous to human nucleolin (Srivastava et al., 1989), whereas p40 and p30 are homologous | | | |
| # to PHAP II and PHAP I, respectively (Vaesen et al., 1994). The 14 amino acid sequence of the peak 29 from p30 corresponds to residues 67 to 80 in PHAP I, with slight differences which are underlined; residues E, I, and F are replaced by D, V, and S, respectively, in the sequence of PHAP I. In peak 33 from p30, the | | | |
| # unknown amino acid referred to as X is a cysteine residue in PHAP I; cysteine residues can not be reveled under the experimental conditions of microsequencing. Peptide 18 from p60 was found to be a mixture of two peptides, the sequences of which (only the first 6 amino acids were sequenced) were differentiated | | | |
| # from each other because of the different concentrations of each peptide. | | | |

B. The NH₂-terminal amino acid sequence of the cell-surface p95 (referred to as p95*) was also carried out as described in the “Experimental Procedures”.

TABLE 4
Inhibition of HIV infection by antisera reacting with the purified
preparation of the V3 loop BPs.

| antiserum | ELISA^{b} (OD 450 nm) | | |
|---|---|---|---|
| Against | | V3 loop-BPs | % Inhibition of |
| Peptide^{a} | Peptide | (p95/p40/p30) | HIV infection^{c} |
| Nucleolin (N) | 1.73 | 0.90 | 72.5% |
| Nucleolin (I) | 1.72 | 0.07 | 13.4% |
| PHAP II (N) | 1.33 | 0.79 | 61.2% |
| PHAP II (I) | 2.14 | 0.19 | 22.3% |
| PHAP I (N) | 2.05 | >3 | 71.2% |
| PHAP I (I) | 1.19 | 0.64 | 61.9% |
| RNP U₁C (I) | 2.26 | >0.05 | 0% |
| ^{a}Rabbit antisera raised against synthetic peptides corresponding to the NH₂-terminal and internal sequences (designated as N and I, respectively) of nucleolin, PHAP II, PHAP I, and RNP U₁C were described in the “Experimental Procedures”. | | | |
| ^{b}ELISA was carried out using either the synthetic peptides corresponding to the NH₂-terminal and internal sequence of nucleolin, PHAP II, and PHAP I (each at 100 ng/ml) and the purified preparation of the V3 loop-BPs (at 200 ng/ml). The reactivitiesat 1:4000 dilutions of each antiserum are given as O.D. values measured at 450 nm. At this serum dilution, an O.D. value less than 0.05 was considered as background. The purified preparation | | | |
| # of the V3 loop-BPs contained p95/nucleolin, p40/PHAP II, and p30/PHAP I as shown in FIGS. 2 and 3. | | | |

^{c}CEM Cells, in (duplicate samples) were infected with the HIV-1 Lai isolate (as in the legend of FIG. 7) in the presence of different rabbit antisera at 300-fold dilution. Virus production was estimated by the concentration of p24 in the culture supematant at 4 days p.i. The % inhibition was calculated by comparison with the production of virus in cells treated with the control rabbit antiserum against U₁C peptide at 300-fold dilution.

TABLE 5
Specificity of the anti-peptide antibodies against nucleolin, PHAP II and PHAP

| antiserum | ELISA^{b} (OD 450 nm) | | | | | | |
|---|---|---|---|---|---|---|---|
| Against | Nucleolin | PHAP II | PHAP I | CXCR4 | RNP U₁C | V3 loop-Bps | |
| peptide^{a} | (peptide) | (peptide) | (peptide) | (peptide) | (peptide) | (p95/p40/p30) | W.B.^{c} |
| Nucleolin (N) | 2.58 | 0.16 | 0.08 | 0.12 | 0.19 | 2.29 | p95 |
| PHAP II (N) | 0.20 | 2.48 | 0.15 | 0.11 | 0.12 | 1.56 | p40 |
| PHAP I (N) | 0.20 | 0.11 | 2.72 | 0.21 | 0.20 | >3 | p30 |
| CXCR4 (N) | 0.16 | 0.15 | 0.14 | >3 | 0.15 | 0.14 | p50 |
| RNP U₁C (I) | 0.12 | 0.16 | 0.18 | 0.16 | >3 | 0.16 | None |
| V3 Loop-BPs | N.T. | N.T. | N.T. | 0.14 | 0.15 | 2.59 | p95/p40/p30 |
| ^{a}Rabbit antisera raised against synthetic peptides corresponding to the NH₂-terminal sequence (N) of nucleolin, PHAP II, PHAP I, CXCR4, and internal (I) sequence of RNP U₁C and the purified preparation of the V3 loop-BPs were as described in the “Experimental Procedures”. These antisera were all active against their proper antigens used to immunize animals, at least at a dilution of 16,000. | | | | | | | |
| ^{b}ELISA was carried out using either the synthetic peptides corresponding to the sequence of nucleolin, PHAP II, PHAP I, CXCR4, RNP U₁C (each at 100 ng/ml) and the purified preparation of the V3 loop-BPs (at 200 ng/ml). The reactivities at 1:2000 dilutions of each serum are given as O.D. values measured at 450 nm. At this dilution of serum, an O.D. value less than 0.3 is considered as background. The purified preparation of the V3 loop-BPs contained | | | | | | | |
| #p95/nucleolin, D/PHAP II, and p30/PHAP I as shown in Figures 2 and 3. | | | | | | | |

^{c}W.B.: Western Immunoblot analysis was carried out using crude cell extracts and the purified preparation of the V3 loop-BPs using 1:100 dilution for each serum (as shown in Figure 3).
N.T., not tested.

TABLE 6
Kinetic rate constants and equilibrium affinity constants of
5(KΨ(CH₂N)PR)TASP and gp 120 to the V3 loop-BPs.

| | | | k_{d} | |
|---|---|---|---|---|
| Binding | | kₐ (× 10⁵)^{a} | (× 10⁻³)^{a} | Kₐ (× 10⁸) |
| Protein(s) | Ligand | M⁻¹S⁻¹ | S⁻¹ | M⁻¹ |
| V3 loop- | 5(KΨ(CH₂N)PR)TASP | 37.50 | 0.39 | 96.1 |
| BPs | 5(KPR)TASP | 3.50 | 2.40 | 1.5 |
| | gp120 HIV-1 Lai | 4.40 | 2.10 | 2.1 |
| | gp120 HIV-1 MN | 1.90 | 0.44 | 4.3 |
| | gp120 HIV-1 SF2 | 0.65 | 0.028 | 23.2 |
| | gp120 HIV-1 SF2 2/3 | 3.80 | 2.40 | 1.6 |
| CD4 | 5(KΨ(CH₂N)PR)TASP | nb^{b} | | |
| | gp120 HIV-1 Lai | | | |
| | gp120 HIV-1 SF2 | | | |
| Relative affinity of 5(KΨ(CH₂N)PR)TASP and g120 fpr the V3 loop-BPs | | | | |
| # was determined using a biosensor instrument (BIAcore). The preparation of the V3 loop-BPs | | | | |
| #containing nucleolin, PHAP II, and PHAP I, was as described in FIG. 2. The gp120 preparations corresponded | | | | |
| # to that of HIV-1 Lai, MN, and SF2 isolate. The gp120 HIV-1 SF2 2/3 represents an nonglycosylated form of gp120. | | | | |
| # The CD4 represented a soluble form of recombinant CD4 generated by the baculovirus expression system. The | | | | |
| # details of the experiment are described in the “Experimental Procedures”. | | | | |

^{a}Association (kₐ) and dissociation (k_{d}) rate constants are the mean values obtained in at least two independent experiments.
^{b}nb, no binding.

TABLE 7
Inhibition of gp120 binding to the V3 loop-BPs by monoclonal antibodies
against the V3 loop.

| | | | Inhibition |
|---|---|---|---|
| mAb | Isotype | Epitope (in gp120)^{a} | (IC₅₀)^{b} |
| AD3 | IgG2a | NH₂-Terminal (aa 1-204) | No Effect^{c} |
| 110C | IgG1, κ | FTD (aa 282-284) | ≈120 nM |
| V3-21 | IgG1, κ | V3: INCTRPN (aa 298-304) | ≈100 nM |
| N11/20 | IgG1, κ | V3: GPGRAFVTI (aa 317-325) | ≈100 nM |
| 110-4 | Not known | V3: (aa 303-323) | ≈100 nM |
| 110-D | IgG2a, κ | (aa 381-394) | No Effect |
| b12 | IgG1 | CD4 binding domain | No Effect |
| ADP390 | IgG2b | CD4 binding domain | No Effect |
| 110-1 | Not known | COOH-terminal (aa 489-511) | No Effect |

The effect of different mAbs on the binding of gp120 to the V3 loop-BPs was determined using biosensor technology. The preparation of the V3 loop-BPs containing nucleolin, PHAP II, and PHAP I, was as described in FIG. 2. The gp120 preparation corresponded to that of HIB-1 Lai isolate. The details of the experiment are described in the “Experimental Procedures”.
^{a}The precise epitope (amino acid residues recognized by mAbs is given when it is known.
^{b}IC₅₀ values represent the concentration of a given mAb to inhibit 50% the binding of gp120 to the V3 loop-BPs.
^{c}No effect was observed at 1:200 fold dilution of the hybridoma culture supernatant containing mAb AD3. The recommended dilution of this antibody in an ELISA test is 1:10,000, and in an immunoblot assay is 1:1000 (Ugen et al., 1993).

TABLE 8
Antibodies against either nucleolin, PHAPII, or PHAP I peptides inhibit
the binding of gp120 to the V3 loop BPs.

| | % Inhibition of gp120 Binding | |
|---|---|---|
| | to the V3 Loop-BPs^{b} | |
| Antisera Against^{a} | Serum at 1:250^{c} | Serum at 1:500^{c} |
| Nucleolin | 89% | 66% |
| PHAP II | 78% | 48% |
| PHAP 1 | 84% | 58% |
| Histone H2B | None | None |
| V3 loop-BPs | 77% | 65% |
| Nucleolin/PHAP II | 65% | None |
| Nucleolin/PHAP I | 60% | None |
| PHAP Il/PHAP I | 91% | 62% |
| Nucleolin/PHAP Il/PHAP I | 76% | 25% |

^{a}Rabbit antisera raised against synthetic peptides corresponding to NH₂-terminal sequence of nucleolin, PHAP II, PHAP I, histone H2B, and the purified preparation of the V3 loop-BPs, were as described in the “Experimental Procedures”. When mixtures of antisera were sued, the dilution of each serum was either 250 or 500 fold as indicated.
^{b}The binding of gp120 to the V3 loop-BPs was investigated by ELISA as described in the legend of FIG. 10.
^{c}The data present the % inhibition of binding at 1:250 and 1:500 dilutions of each serum^{c}.

TABLE 9
Kinetic rate and equilibrium affinity constants of 5[KΨy(CH₂N)PR]-TASP and
gp120 to the V3-BPs and to CD4.

| | | kₐ (× 10⁵)* | k_{d} (× 10⁻³)* | Kₐ (× 10⁸) |
|---|---|---|---|---|
| Binding Protein(s) | Ligand | M⁻¹S⁻¹ | S⁻¹ | M⁻¹ |
| V3-BPs | 5[KΨ(CH₂N)PR]-TASP | 37.50 | 0.39 | 96.1 |
| 5[KPR]-TASP | 3.50 | 2.40 | 1.5 | |
| gp120 HIV-1 Lai | 4.40 | 2.10 | 2.1 | |
| gp120 HIV-1 MN | 1.90 | 0.44 | 4.3 | |
| gp120 HIV-1 SF2 | 0.65 | 0.03 | 23.2 | |
| gp120 HIV-1 SF2 | 3.80 | 2.40 | 1.6 | |
| | 2/3 | | | |
| gp41 HIV-1 Lai | No binding | — | — | |
| CD4 | 5[KΨ(CH₂N)PR]-TASP | No binding | — | — |
| gp120 HIV-1 Lai | 5.88 | 54.50 | 10.8 | |
| gp120 HTV-1 MN | 2.98 | 38.60 | 7.7 | |
| gp120 HIV-1 SF2 | 11.50 | 5.60 | 2.1 | |
| gp41 HIV-1 Lai | No binding | — | — | |

Relative affinity of 5[KΨ(CH₂N)PR]-TASP and gp120 for the V3-BPs and CD4 was determined using a biosensor instrument (BIAcore, Materials and Methods). The preparation of the V3-BPs containing nucleolin, PHAP II, and PHAP I, was as described in FIG. 1. The gp120 preparations corresponded to that of lymphotropic HIV-1 isolates Lai, MN, and SF2. The gp120 HIV-1 SF2 2/3 represents an unglycosylated form of gp120 SF2. The CD4 represented a soluble form of recombinant CD4.
*Association (kₐ) and dissociation (k_{d}) rate constants are the mean values obtained in at least two independent experiments.

TABLE 10
Inhibition of gp120 binding to the V3-BPs by monoclonal antibodies
against the V3 loop.

| | | | Inhibition |
|---|---|---|---|
| mAb | Isotype | Epitope (in gp120)^{a} | (IC₅₀)^{b} |
| AD3 | IgG2a | NH2-Terminal (aa 1-204) | No Effect^{c} |
| 110-C | IgG1, κ | FTD (aa 282-284) | ≈120 nM |
| V3-21 | IgG1, κ | V3: INCTRPN (aa 298-304) | ≈100 nM |
| N11-20 | IgG1, κ | V3 GPGRAFVTI (aa 317-325) | ≈100 nM |
| 110-4 | Not known | V3: (aa 303-323) | ≈100 nM |
| 110-D | IgG2a, κ | (aa 381-394) | No Effect |
| b12 | IgG1 | CD4 binding domain | No Effect |
| ADP390 | IgG2b | CD4 binding domain | No Effect |
| 110-1 | Not known | COOH-terminal (aa 489-511) | No Effect |

The effect of different mAbs on the binding of gp120 Lai to the V3-BPs was determined using biosensor technology (Materials and Methods). The preparation of the V3-BPs containing nucleolin, PHAP II, and PHAP I, was as described in FIG. 1.
^{a}The precise epitope (amino acid residues) recognized by mAbs is given when it is known. The amino acid residue numbers were according to the sequence of HIV-1 Lai.
^{b}IC₅₀ values represent the concentraiton of a given mAb to inhibit 50% the binding of gp120 (800 nM) to the V3-BPs (100 ng/ml). Besides mAb b12 which is a human mAb, all the other mAbs were of murine origin.
^{c}No effect was observed at a 1:200 fold dilution of the hybridoma culture supernatant containing mAb AD3. The recommended dilution of this antibody in an ELISA test is 1:10,000, and in an immunoblot assay is 1:1000 (55).

The effect of different mAbs on the binding of gp120 Lai to the V3-BPs was determined using biosensor technology (Materials and Methods). The preparation of the V3-BPs containing nucleolin, PHAP II, and PHAP I, was as described in 1 +L.

TABLE 11
Inhibition of HIV entry by 5[KΨPR]TASP constructs.

| | Cells | | IC₅₀ |
|---|---|---|---|
| Pseudopeptides | HeLa | Virus Isolate | (μM) |
| 5[KΨPR]-TASP* | P4 | HIV-1 Lai | 0.5 |
| 5[KΨPR]-TASP | P4 | HIV-1 Lai | 0.1 |
| 5[QPQ]-TASP | P4 | HIV-1 Lai | N.E. |
| 5[KER]-TASP | P4 | HIV-1 Lai | N.E. |
| 5[KΨPR]-TASP | P4-C5 | HIV-1 Lai | 0.1 |
| 5[QPQ]-TASP | P4-C5 | HIV-1 Lai | N.E. |
| 5[KΨPR]-TASP* | P4-C5 | HIV-1 Bal | 0.8 |
| 5[KΨPR]-TASP | P4-C5 | HIV-1 Bal | 0.3 |
| 5[QPQ]-TASP | P4-C5 | HIV-1 Bal | N.E. |
| 5[KER]-TASP | P4-C5 | HIV-1 Bal | N.E. |
| 5[KΨPR]-TASP | P4-C5 | HIV-1 JRCSF | 0.3 |
| 5[QPQ]-TASP | P4-C5 | HIV-1 JRCSF | N.E. |
| 5[KΨPR]-TASP | P4-C5 | HIV-1 89.6 | 0.3 |
| 5[QPQ]-TASP | P4-C5 | HIV-1 89.6 | N.E. |
| 5[KΨPR]-TASP* | P4-C5 | HIV-1 UGO29A | 0.3 |
| 5[QPQ]-TASP | P4-CS | HIV-1 UGO29A | N.E. |
| 5[KΨPR]-TASP* | P4-CS | HIV-2 ROD | 0.4 |
| 5[KΨPR]-TASP | P4-CS | HIV-2 ROD | 0.2 |
| 5[QPQ]-TASP | P4-CS | HIV-2 ROD | N.E. |
| 5[KΨPR]-TASP | P4-CS | HIV-2 CBL | 2 μM |
| 5[QPQ]-TASP | P4-CS | HIV-2 CBL | N.E. |
| 5[KΨPR]-TASP* | P4 | VSV/HIV | N.E. |
| 5[KΨPR]-TASP | P4 | VSV/HIV | N.E |

Two different cell clones, HeLa P4 and HeLa P4-C5 expressing human CD4 and human CD4 and CCR5 molecules, respectively, were infected with the different HIV-1 and HIV-2 isolates as indicated. Both of these cell clones, express also the bacterial lacZ gene under the control of HIV-1 LTR (cells were obtained from O. Schwartz and P. Charneau, Institut Pasteur). Entry of HIV and replication, results in the activation of the HIV LTR, leading to the expression of the lacZ gene. At 24 and 48 hours post-infection, the β-galactosidase activity could be measured in cell extracts directly. Thus, the β-galactosidase activity could be used to monitor HIV entry. The 5[KψPR]-TASP* construct refers to the previously described molecule. The 5[KψPR]-TASP construct was as had been described before but the proline residue was dehydroxyproline. This latter molecule manifests a higher inhibitory activity. The constructs 5[QPQ]-TASP and 5[KER]-TASP represent control peptides which do not affect HIV infection. VSV/HIV represents HIV-1 pseudotyped with VSV envelope glycoproteins and was generously provided by O. Schwartz. N.E. No effect on HIV entry.

### REFERENCES

Adra et al., 1987, Gene, 60:65-74

Alizon, M., and Dragic, T. (1994). CD26 antigen and HIV fusion, Science 264, 1161-1162.

Avril, L.-E., Di Martino-Ferrer, M., Pignede, G., Séman, M., and Gauthier, F. (1994). Identification of the U-937 membrane-associated proteinase interacting with the V3 loop of HIV-1 gp120 as cathepsin G. FEBS Letters 345, 81-86.

Bacharach E. et al., 1997, The carboxyterminal portion of nucleolin binds to the moloney MuLV nucleocapsid and inhibits virion production. Abstract. Cold Spring Harbor Laboratory.

Barany F., 1911, Proc. Natl. Acad. Sci. USA, 88:189-193.

Barchan D. et al., 1995, J. Immunol., 155(9):4264-4269.

Battini J. L. et al., 1995, J. Virol., 70: 4387-4393.

Beard et al., 1980, Virology, 75:81

Belenguer, P., Caizergues-Ferrer, M., Labbé, J.-C., Dorée, M., and Amalric, F. (1990). Mitosis-specific phosphorylation of nucleolin by p34^{cdc2} protein kinase. Molec. Cell. Biol. 10, 3607-3618.

Benkirane, N., Guichard, G., Van Regenmortel, M. H. V., Briand, J. P., and Muller, S. (1995). Cross-reactivity of antibodies to retro-inverso peptidomimetics with the parent protein histone H3 and chromatin core particle. J. Biol. Chem. 270, 11921-11926.

Bhat, S., Spitalnik, S. L., Gonzalez-Scarano, F., and Silberberg, D. H. (1991). Galactosyl ceramide or a derivative is an essential component of the neural receptor for human immunodefiency virus type 1 envelope glycoprotein gp120. Proc. Natl. Acad. Sci. USA 88, 7131-7134.

Blanco, J., Marié, I., Callebaut, C., Jacotot, E., Krust, B. & Hovanessian, A. G. (1996) Exp. Cell Res., 225, 102-111.

Bolmont et al., J. of Submicroscopic cytology and pathology, 1990, 22:117-122;

Borer, R. A., Lehner, C. F., Eppenberger, H. M., and Nigg, E. A. (1989). Major nucleolar proteins shuttle between nucleus and cytoplasm. Cell 56, 379-390.

Borman A. M. et al., 1995, J. Virol., 69: 2058-2067.

Borrow, P., and Oldstone, M. B. A. (1992). Characterization of lymphocytic choriomeningitis virus-binding proteins: a candidate cellular receptor for the virus. J. Virol. 66, 7270-7281.

Bouche, G., Caizergues-Ferrer, M., Bugler, B., and Amalric, F. (1984). Interrelations between the maturation of a 100 kDa nucleolar protein and prerRNA synthesis in CHO cells. Nucl. Acids Res. 12, 3025-3035.

Bour S. et al., 1995, Microbiological reviews, 59: 63-93.

Bowie J. U. et al., 1990, Science, 47:1306-1310.

Briand, J. P., Barin, C., Van Regenmortel, M. H., and Muller, S. (1992). Application and limitations of the multiple antigen peptide (MAP) system in the production and evaluation of anti-peptide and anti-protein antibodies. J. Immunol. Methods 156, 255-265.

Broder, C., Nussbaum, O., Gutheil, W. G., Bachovchin, W. W., Berger, E. A., Patience, C., McKright, A., Clapham, P. R., Boyd, M. T., Weiss, R. A., Schulz, T. F., Camerini, D., Planelles, V., Chen, I. S. Y., Alizon, M., and Dragic, T. (1994). CD26 antigen and HIV fusion? (Technical Comments) Science 264, 1156-1159.

Bugler, B., Caizergues-Ferrer, M., Bouche, G., Bourbon, H., and Amalric, F. (1982). Detection and localization of a class of proteins immunologically related to a 100-kDa nucleolar protein. Eur. J. Biochem. 128, 475-480.

Burton, D. R., Barbas, C. F., Persson, M. A. A., Koenig, S., Chanock, R. M., and Lerner, R. A. (1991). A large array of human monoclonal antibodies to HIV-1 from combinatorial libraries of asymptomatic seropositive individuals. Proc. Natl. Acad. Sci. USA 88, 10134-10137.

Callebaut, C., and Hovanessian, A. G. (1996). CD26 and HIV infection. Res. Virol. 147, 67-69.

Callebaut, C., Jacotot, E., Blanco, J., Krust, B., and Hovanessian, A. G. (1997). The level of CD26 determines the rate of HIV entry in a CD4+ T cell line. In Cellular Peptidases in Immune Functions and Disease., S. Ansorge and J. Langner, eds.: Plenum Publishing Corporation.

Callebaut, C., Jacotot, E., Guichard, G., Krust, B., Rey-Cuille, M. A., Cointe, D., Benkirane, N., Blanco, J., Muller, S., Briand, J. P., and Hovanessian, A. G. (1996). Inhibition of HIV infection by pseudopeptides blocking viral envelope glycoprotein-mediated membrane fusion and cell death. Virology 218, 181-192.

Callebaut, C., Jacotot, E., Krust, B., and Hovanessian, A. G. (1994). CD26 Antigen and HIV Fusion? Science 264, 1156-1165.

Callebaut, C., Jacotot, E., Marié, I., Blank, U., Robert, N., Krust, B., and Hovanessian, A. (1994). The role of CD26 in HIV infection: viral entry and its cytopathic effect. In IXé Colloque des Cent Gardes, M. Girard and B. Dodet, eds.: In Retrovirus of Human AIDS and related diseases, pp. 141-149.

Callebaut, C., Krust, B., Jacotot, E., and Hovanessian, A. G. (1993). T cell Activation Antigen, CD26, as a Cofactor for Entry of HIV in CD4⁺ cells. Science 262, 2045-2050.

Callebaut C. et al, 1997a, J. Biol. Chem., 272: 7159-7166.

Camerini, D., Planelles, V., and Chen, I. S. Y. (1994). CD26 antigen and HIV fusion ? Science 264, 1160-1161.

Castagnoli L. et al. (Felici F.), 1991, J. Mol. Biol., 222:301-310.

Chai H. et al., 1993, Biotechnol. Appl. Biochem., 18:259-273.

Chee et al., 1996, Science, 274:610-614.

Chen, Y.-H., Ebenbichier, C., Vomhagen, R., Schulz, T. F., Steindi, F., Bock, G., Katinger, H., and Dierich, M. P. (1992). HIV-1 gp41 contains two sites for interaction with several proteins on the helper T-lymphoid cell line, H9. AIDS 6, 533-539.

Chin, L. T., Duenas, M., Levi, M., Hinkula, J., Wahren, B., and Borrebaeck, C. A. K. (1995). Molecular characterization of a human anti-HIV 1 monoclonal antibody revealed a CD26-related motif in CDR2. Immunology Letters 44, 25-30.

Choe, H., Farzan, M., Sun, Y., Sullivan, N., Rollins, B., Ponath, P. D., Wu, L., Mackay, C. R., La rosa, G., Newman, W., Gerard, N., Gerard, C., and Sodroski, J. (1996). The beta-chemokine receptors CCR3 and CCR5 facilitate infection by primary HIV-1 isolates. Cell 85, 1135-1148.

Cocchi, F., DeVico, A. L., Garzino-Demo, A., Cara, A., Gallo, R. C., and Lusso, P. (1996). The V3 domain of the HIV-1 gp120 envelope glycoprotein is critical for chemokine-mediated blockade of infection. Nature Medicine 2(11), 1244-1247.

Corbeau, P., Olive, D., and Devaux, C. (1991). Anti-HLA class-I heavy chain monoclonal antibodies inhibit human immunodeficiency virus production by peripheral blood mononuclear cells. Eur. J. Immunol. 21, 865-871.

Curtis, B. M., Schamowske, S., and Watson, A. J. (1992). Sequence and expression of a C-type lectin that exhibits CD4-independent binding of human immunodeficiency virus envelope glycoprotein gp120. Proc. Natl. Acad. Sci. USA 89, 8356-8360.

D'Souza, M. P., and Harden, V. A. (1996). Chemokines and HIV-1 second receptors. Nature Medicine 2(12), 1293-1300.

Dawson, P. E, and Kent, S. B. H. (1993). Convenient total synthesis of a 4-Helix TASP molecule by chemoselective ligation. J. of the American Chemical Society 115, 7263-7266.

Dean et al., Nature Medicine, 2:1240-1243.

Deckert P. M. et al., 1996, J. Immunol., 156(2):826-833.

Deng, H., Liu, R., Ellmeier, W., Choe, S., Unutmaz, D., Burkhart, M., Di Marzio, P., Marmon, S., Sutton, R. E., Hill, C. M., Davis, C. B., Peiper, S. C., Schall, T. J., Littman, D. R., and Landau, N. R. (1996). Identification of a major co-receptor for primary isolates of HIV-1. Nature 381, 661-666.

Doranz, B. J., Rucker, J., Yi, Y., Smyth, R. J., Samson, M., Peiper, S. C., Parmentier, M., Collman, R. G., and Doms, R. W. (1996). A dual-tropic primary HIV-1 isolate that uses fusin and the beta-chemokine receptors CKR-5, CKR-3 and CKR-2b as fusion cofactors. Cell 85, 1149-1158.

Dragic, T., Litwin, V., Allaway, G. P., Martin, S. R., Huang, Y., Nagashima, K. A., Cayanan, C., Maddon, P. J., Koup, R. A., Moore, J. P., and Paxton, W. A. (1996). HIV-1 entry into CD4+ cells is mediated by the chemokine receptor CC-CKR-5. Nature 381, 667-673.

Dragic, T., Picard, L., and Alizon, M. (1995). Proteinase-resistant factors in human erythrocyte membranes mediate CD4-dependent fusion with cells expressing human immunodeficiency virus type 1 envelope glycoproteins. J. Virol. 69, 1013-1018.

Duck P. et al., 1990, Biotechniques, 9:142-147.

Dukes, C. S., Yu, Y., Rivadeneira, E. D., Sauls, D. L., Liao, H.-X., Haynes, B. F., and Weinberg, J. B. (1995). Cellular CD44S as a determinant of human immunodeficiency virus type 1 infection and cellular tropism. J. Virol. 69, 4000-4005.

Earnshaw, W. C. (1987). Anionic regions in nuclear proteins. J. Cell Biol. 105, 1479-1482.

Ebenbichler, C. F., Roder, C., Vormhagen, R., Ratner, L., and Dierich, M. P. (1993). Cell surface proteins binding to recombinant soluble HIV-1 and HIV-2 transmembrane proteins. AIDS 7,489-495.

Ezan E. et al., 1986, Regulatory peptides, 14:155-167.

Ezan E. et al., 1996, Ther. Drug Monitoring.

Fang, S.-H., and Yeh, N.-H. (1993). The self-cleaving activity of nucleolin determines its molecular dynamics in relation to cell proliferation. Exp. Cell Res. 208, 48-53.

Fankhauser C. et al, 1991, Mol. and Cell. Biol., 11: 2567-2575.

Fantini, J., Cook, D. G., Nathanson, N., Spitalnik, S. L., and Gonzalez-Scarano, F. (1993). Infection of colonic epithelial cell lines by type 1 human immunodeficiency virus is associated with cell surface expression of galactosylceramide, a potential alternative gp120 receptor. Proc. Natl. Acad. Sci. USA 90,2700-2704.

Feldman and Steg, 1996, Medecine/Sciences, synthese, 12:47-55.

Felgner et al;, 1987, Proc. Natl. Acad. Sci., 84:7413

Feng, Y., Broder, C. C., Kennedy, P. E., and Berger, E. A. (1996). HIV-1 entry cofactor: functional cDNA cloning of a seven-transmembrane G protein-coupled receptor. Science 272, 872-876.

Fraley et al, 1980, J. Biol. Chem., 255:10431

Freed, E. O., Myers, D. J., and Risser, R. (1991). Identification of the principal neutralizing determinant of human immunodeficiency virus type 1 as a fusion domain. J. Virol. 65, 190-194.

Fuller S. A. et al., 1996, Immunology in Current Protocols in Molecular Biology, Ausubel et al. Eds, John Wiley & Sons, Inc., USA.

Graham, 1984, EMBO J., 3:2917

Guateli J. C. et al., 1990, Proc. Natl. Acad. Sci. USA, 87:1874-1878.

Harouse, J. M., Bhat, S., Spitalnick, S. L., Laughlin, M., Stefano, K., Silbergberg, D. H., and Gonzalez-Scarano, F. (1991). Inhibition of entry of HIV-1 in neural cell lines by antibodies against galactosyl ceramide. Science 253, 320-323.

Harrop, H. A., Coombe, D. R., and Rider, C. C. (1994). Heparin specifically inhibits binding of V3 loop antibodies to HIV-1 gp120, an effect potentiated by CD4 binding. AIDS 8, 183-192.

Hattori, T., Koito, A., Takatsuki, K., Kido, H., and Katunuma, N. (1989). Involvement of tryptase-related cellular protease(s) in human immunodeficiency virus type 1 infection. FEBS Letters 248, 48-52.

Henderson, L. A., and Quershi, M. N. (1993). A peptide inhibitor of human immunodeficiency virus infection binds to novel human cell surface polypeptides. J. Biol. Chem. 268, 15291-15297.

Hildreth, J. E. K., and Orentas, R. J. (1989). Involvement of a leukocyte adhesion receptor (LFA-1) in HIV-induced syncytium formation. Science 244, 1075-1079.

Hook W. A. et al., 1991, J. Immunol., 147: 2670-2676.

Houbenweyl, 1974, in Meuthode der Organischen Chemie, E. Wunsch Ed., Volume 15-I et 15-II, Thieme, Stuttgart.

Hovanessian, A. G. (1994). Apoptosis in HIV infection: the role of extracellular and transmembrane glycoproteins. In Apoptosis II: the Molecular Basis of Apoptosis in Disease, D. L. Tomei and F. O. Cope, eds.: Cold Spring Harbor Laboratory Press), pp. 21-42.

Huygen et al., 1996, Nature Medicine, 2(8):893-898.

Jacotot, E., Callebaut, C., Blanco, J., Krust, B., Neubert, K., Barth, A., and Hovanessian, A. G. (1996a). Dipeptidyl peptidase IV-b: a novel form of cell-surface expressed protein with dipeptidyl peptidase IV activity. Eur. J. Biochem. 239, 248-258.

Jacotot, E., Callebaut, C., Blanco, J., Riviere, Y., Krust, B., and Hovanessian, A. G. (1996). HIV envelope glycoprotein-induced cell killing by apoptosis is enhanced with increased expression of CD26 in CD4⁺ T cells. Virology 223, 318-330.

Javaherian, K., and McDanal, C. (1995). Random copolymers containing specific ratios of negatively charged and aromatic amino acids bind V3 disulfide loop and neutralize diverse HIV type 1 isolates. AIDS Res. Hum. Retroviruses 11, 1163-1168.

Javaherian, K, Khiroya, R, Quinlan, C., Boyd, J. G., and Hanham, K. A. (1994). V3-mediated heparin neutralization of HIV type 1. AIDS res. & Hum. Retrovirases 10, 1421-1423.

Jordan, P., Heid, H., Kinzel, V., and Kübler, D. (1994). Major cell surface-located protein substrates of an ecto-protein kinase are homologs of known nuclear proteins. Biochemistry 33, 14696-14706.

Kaneda et al., 1989, Science, 243:375.

Kido, H., Kukutomi, A., and Katunuma, N. (1990). A novel membrane bound serine-esterase in human T4+ lymphocytes immunologically reactive with antibody inhibiting syncytia induced by HIV-1: purification and characterization. J. Biol. Chem. 265, 21979-21985.

Kievitis T. et al., 1991, J. Virol. Methods, 35:273-286.

King, D. S., Fields, C. G., and Fields, G. B. (1990). A cleavage method which minipizes side reactions following Fmoc solid phase peptide synthesis. Int. J. Pept. Protein Res. 36, 255-266.

Kinney-Thomas, E., Weber, J. N., McClure, J., Clapham, P. R., Singhal, M. C., Shriver, M. K., and Weiss, R. (1988). Neutralising monoclonal antibodies to the AIDS virus. AIDS 2, 25-29.

Kleinman, H. K., Weeks, B. S., Cannon, F. B., Sweeney, T. M., Sephel, G. C., Clement, B., Zain, M., Olson, M. O. J., Jucker, M., and Burrous, B. A. (1991). Identification of a 110 kDa nonintegrin cell surface laminin-binding protein which recognizes an a chain neunrte-promoting peptide. Arch. Biochem. Biophysics 290, 320-325.

Koch Y., 1977, Biochem. Biophys. Res. Commun., 74:488-491.

Koito, A., Hattori, T., Murakami, T., Matsushita, S., Maeda, Y., Yamamoto, T., and Takatsuki, K. (1989). A neutralizing epitope of human immunodeficiency virus type 1 has homologous amino acid sequences with the active site of inter-a-trypsin inhibitor. Intern. Immunol. 1, 613-618.

Kort et al., 1983, Nucleic Acids Research, 11:8287-8301.

Krantz, S., Salazar, R., Brandt, R., Kellermann, J., and Lottspeich, F. (1995). Purification and partial amino acid sequencing of a fructosyllysine-specific binding protein from cell membranes of the monocyte-like cell line U937. Biochem. Biophys. Acta 1266, 109-112.

Krust, B., Callebaut, C. & Hovanessian, A. G. (1993) AIDS Res. Hum. Retroviruses 11, 1087-1090.

Krust, B., Callebaut, C., and Hovanessian, A. G. (1993). Inhibition of entry of HIV in cells by poly(A).poly(U). AIDS Res. Hum. Retroviruses 9, 1087-1090.

Kwoh D. Y. et al., 1989, Proc. Natl. Acad. Sci. USA, 86:1173-1177.

Laman, J. D., Schellekens, M. N., Abacioglu, Y. F., Lewis, G. K., Tersmette, M., Fouchier, R. A. M., Langedijk, J. P. M., Claasen, E., and Boersman, W. J. A. (1992). Variant-specific monoclonal and group-specific polyclonal human immunodeficiency virus type 1 neutralizing antibodies raised with synthetic peptides from the gp120 third variable domain. J. Virol. 66, 1823-1831.

Landegren U. et al., 1988, Science, 241:1077-1080.

Lapham, C. K., Ouyang, J., Chandrasekhar, B., Nguyen, N. Y., Dimitrov, D. S., and Golding, H. (1996). Evidence for cell-surface association between fusin and the CD4-gp120 complex in human cell lines. Science 274, 602-605.

Lasky, L. A., Nakamura, G., Smith, D. H., Fennie, C., Shimasaki, C., Patzer, E., Berman, P., Gregory, T., and Capon, D. J. (1987). Delineation of a region of the human immunodeficiency virus type 1 gp120 glycoprotein critical for interaction with the CD4 receptor. Cell 50, 975-85.

Laurent-Crawford, A. G., and Hovanessian, A. G. (1993). The cytopathic effect of human immunodeficiency virus is independent of high levels of unintegrated viral DNA accumulated in response to superinfection of cells. J. Gen. Virol. 74, 2619-2628.

Laurent-Crawford, A. G., Coccia, E., Knist, B., and Hovanessian, A. G. (1995). Membrane-expressed HIV envelope glycoprotein hererodimer is a powerful inducer of cell death in uninfected CO4+ target cells. Res. Virol. Institut Pasteur 146, 5-17.

Lenhard T. et al., 1996, Gene, 169:187-190.

Levrero et al, 1991, Gene, 101:195

Leydet, A., El Hachemi, H., Boyer, B., Lamaty, G., Roque, J. P., Schols, D., Snoeck, R., Andrei, G., Ikeda, S., Neyts, J., Reymen, D., Este, J., Witvrouw, M., and De Clercq, E. (1996). Polyanion inhibitors of human immunodeficiency virus and other viruses. Part 2. Polymerized anionic surfactants derived from amino acids and dipeptides. J. Med. Chem. 39, 1626-1634.

Li Y. P. et al., 1996, Eur. J. Biochem., 237: 153-158.

Li Y. P. et al., 1997, J. Virol., 71: 4098-4102.

Linsley, P. S., Ledbetter, J. A., Kinney-Thomas, E., and Hu, S. L. (1988). Effects of anti-gp120 monoclonal antibodies on CD4 receptor binding by the env protein of human immunodeficiency virus type 1. J. Virol. 62, 3695-3702.

Liu et al., 1996, Nature, 382:722-725.

Lucas A. H., 1994, In: Development and Clinical Uses of Haempophilus b

Conjugate vaccines. New Approaches to Polysaccharide vaccines, pp. 467-469. Mann, D. L., Read-Connole, E., Arthur, L., Robey, W. G., Wernet, P., Schneider, E. M., Blattner, W. A., and Popovic, M. (1988). HLA-DR is involved in the HIV-1 binding site on cells expressing MHC class-II antigens. J. Immunol. 141, 1131-1136.

Maran A. et al., 1994, 265:789-792.

Marasco W. A. et al., 1994, Arch. Virol., 139: 133-154.

Martin M. et al., 1989, J. Immunol., 155: 133-154.

Matsuoka, K., Taoka, M., Satozawa, N., Nakayama, H., Ichimura, T., Takahashi, N., Yamalcuni, T., Song, S.-Y., and Isobe, T. (1994). A nuclear factor containing the leucine-rich repeats expressed in murine cerebellar neurons. Proc. Natl. Acad. Sci. USA 91, 9670-9674.

Mbemba, E., Carré, V., Atemezem, A., Saffar, L., Gluckman, J. C., and Gattegno, L. (1996). Inhibition of human immunodeficiency virus infection of CD4+cells by CD4-Free glycopeptides from monocytic U937 cells. AIDS Res. & Human Retroviruses 12, 47-53.

McKeating, J. A., Griffiths, P. D., and Weiss, R. A. (1990). HIV susceptibility conferred to human fibroblasts by cytomegalovirus-induced Fc receptor. Nature (London) 343, 659-661.

McKeating, J. A., Thali, M., Furman, C., Karwowska, S., Gorny, M. K., Cordell, J., Zolla-Pazner, S., Sodroski, J., and Weiss, R. A. (1992). Amino acid residues of the human immunodeficiency virus type 1 gp120 critical for the binding of rat and human neutralizing antibodies that block the gp120-sCD4 interaction. Virology 190, 134-142.

Mebatsion T. et at., 1997, Cell, 90: 841-847.

Merrifield, 1963, J. Am. Chem. Soc., 85:2149-2154.

Midoux, 1993, Nucleic Acids Research, 21:871-878.

Mizukami, T., Fuerst, T. R., Berger, E. A., and Moss, B. (1988). Binding region for human immunodeficiency virus (HIV) and epitopes for HIV-blocking monoclonal antibodies of the CD4 molecule defined by site-directed mutagenesis. Proc. Natl. Acad. Sci. USA 85, 9273-9277.

Moore, J. P., and Nara, P. L. (1991). The role of the V3 loop of gp120 in HIV infection. AIDS 5, S21-S33.

Moore, J. P., Jameson, B. A, Weiss, R. A., and Sattentau, Q. J. (1993). The HIV-cell fusion reaction. In Viral Fusion mechanisms, J. (Bentz, ed.: CRC Press, Boca Raton, Fla.), pp. 233-289.

Myers, G., Korber, B., Wain-Hobson, S., Jeang, K.-T., Henderson, L. E., and Pavlakis, G. N. (1994). Human Retroviruses and AIDS 1994, L. A. Theoretical Biology and Biophysics Group T-10. Los Alamos National Laboratory, USA., ed.

Naif et al., 1998 [Ref Biblio à compléter S.V.P.]

Neimark, J., and Briand, J. P. (1993). Development of a fully automated multichannel peptide synthesizer with integrated TFA cleavage capability. Pept. Res. 6, 219-228.

Niwa, Y., Yano, M., Futaki, S., Okumura, Y., and Kido, H. (1996). T-cell membrane-associated serine protease tryptase TL2, binds human immunodeficiency virus type 1 gp120 and cleaves third-variable-domain loop of gp120 neutralizing antibodies of human immunodeficiency virus type 1 inhibit cleavage of gp120. Eur. J. Biochem. 237, 64-70.

O'Reilly et al., 1992, Baculovirus expression vectors: a Laboratory Manual. W. H. Freeman and Co., New York.

Oberlin E. et al., 1996, Nature, 382: 833-835.

Ohno et al., 1994, Sciences, 265:781-784.

Ohwada A. et al., 1996, 88(3):778-784.

Oldenburg K. R. et al., 1992, Proc. Natl. Acad. Sci., 89:5393-5397.

Oravecz, T., Pall, M., and Norcross, M. A. (1996). b-chemokines inhibition of monocytotropic HIV-1 infection. Interference with a postbinding fusion step. J. Immunol. 157, 1329-1332.

Oravecz, T., Roderiquez, G., Koffi, 3., Wang, J., Ditto, M., Bou-Habib, D. C., Lusso, P., and Norcross, M. A. (1995). CD26 expression correlates with entry, replication and cytopathicity of monocytotropic HIV-1 strains in a T-cell line. Nature Medicine 1, 919-926.

Oravecz et al., 1996, J. Immunol., 157: 1329-1332.

Osmanov, S., Heyward, W. L., and Esparza, J. (1994). The World Health Organization Network for HIV isolation and characterization: summary of a pilot study. AIDS Res. Hum. Retroviruses 10, 1325-1326.

Pagano et al., 1967, J. Virol., 1:891.

Pantaleo, G., Butini, L., Graziosi, C., Poli, G., Schnittman, S. M., Greenhouse, J. J., Gallin, J. I., and Fauci, A. S. (1991a). Human immunodeficiency virus (HIV) infection in CD4+ T lymphocytes genetically deficient in LFA-1:LFA-1 is required for HIV-mediated cell fusion but not for viral transmission. J. Exp. Med. 173, 511-514.

Pantaleo, G., Poli, G., Butini, L., Fox, C., Dayton, A. I., and Fauci, A. S. (1991b). Dissociation between syncytia formation and HIV spreading. Suppression of syncytia formation does not necessarily reflect inhibition of HIV infectin. Eur. J. Immunol. 21, 1771-1774.

Parmley and Smith, Gene, 1988, 73:305-318.

Pastore, 1994, Circulation, 90:1-517.

Patel, M., Yanagishita, M., Roderiquez, G., Chequer, D., Bou-Habib, T., Oravecz, T., HascaHl, V. C., and Norcross, M. A. (1993). Cell-surface heparan sulfate proteoglycan mediates HIV-1 infection of T-cell lines. AIDS Res. Hum. Retroviruses 9, 167-174.

Patience, C., McKnight, A., Clapham, P. R., Boyd, M. T., Weiss, R. A., and Schulz, T. F. (1994). CD26 antigen and HIV fusion ? Science 264, 1159-1160.

Perosa F. et al., 1996, J. Immunol., 156(9):3563-3569.

Pfeifle, J., and Anderer, A. (1983). Isolation and characterization of phosphoprotein pp105 from simian virus 40-transformed mouse fibroblasts. Biochem. Biophys. Acta 762, 86-93.

Pfeifle J., and Anderer, F. A. (1983). Localization of phosphoprotein PP105 in cell lines of various species. Biochem. Biophys. Res. Comm. 116, 106-112.

Pfeifle, J., Hagmann, W., and Anderer, F. A. (1981). Cell adhesion-dependent differences in endogenous protein phosphorylation on the surface of various cell lines. Biochim. Biophys. Acta 670, 274284.

Polonelli et al., 1996, J. Immunol., 156(5): 1880-1885/

Ptashne, M. (1988). How eukaryotic transcriptional activators work. Nature 335, 683-689.

Qureshi, N. M., Coy, D. H., Garry, R. F., and Henderson, L. A. (1990). Characterization of a putative cellular receptor for HIV-1 transmembrane glycoprotein using synthetic peptides. AIDS 4, 553-558.

Raab de Verdugo, U., Selinka, H.-C., Huber, M., Kramer, B., Kellermann, J., Hofschneider, P. H., and Kandolf, R. (1995). Characterization of a 100-kilodalton binding protein for the six serotypes of coxsackie B viruses. J. Virol. 69, 6751-6757.

Rao, P. R., Talle, M. A., Kung, P. C., and Goldstein, G. (1983). Five epitopes of a differentiation antigen on human inducer T cells distinguished by monoclonal antibodies. Cell. Immunol. 80, 310-319.

Rettlez et al., 1987, Mol. Cell. Biol., 7:1676-1685.

Robinson J. R. et al., 1988, In :Biological approaches to the controlled delivery of drugs, Juliano R. L. (Ed.), Annals of the New York Academy of Sciences, 507:307-314.

Roth J. A. et al., 1996, Nature Medicine, 2(9):985-991.

Rougeot, C., I. Rosinski-Chupin, E. Njamkepo, and F. Rougeon., Eur. J. Biochem. 219 (3): 765-773, 1994.

Saiki, R. K. et al. 1985. Science. 230:1350-1354.

Sambrook, J. et al. 1989. In Molecular cloning : A Laboratory Manual. Cold Spring Harbor, N.Y.: Cold Spring Harbor Laboratory Press.

Sanchez-Pescador R., 1988, J. Clin. Microbiol., 26(10):1934-1938.

Sato, A. I., Balamuth, F. B., Ugen, K. E., Williams, W. V., and Weiner, D. B. (1994). Identification of CD7 glycoprotein as an accessory molecule in HIV-1 mediated syncytium formation and cell free infection. J. Immunol. 152, 5142-5152.

Saunal, H., Karlson, R, and Van Regenmortel, M. H. V. (1996). In immununochemistry 2. In A practical Approach, A. P. and M. W. Turner, eds. (Oxford University Press.

Schnell M. J. et al., 1997, Cell, 90: 849-857.

Schwartz O. et al., 1996, Cell, 90 : 849-857.

Sczakiel G. et al., 1995, Trends Microbiol., 1995, 3(6):213-217.

Segev D., 1992, in <<Non-radioactive Labeling and Detection of Biomolecules>>. Kessler C. Springer Verlag, Berlin, New-York, 197-205.

Shen T. Y., 1988, 1988, In :Biological approaches to the controlled delivery of drugs, Juliano R. L. (Ed.), Annals of the New York Academy of Sciences, 507:272-280.

Smith et al., 1983, Mol. Cell. Biol., 3:2156-2165.

Sommerfelt, M. A., and Asjö, B. (1995). Intercellular adhesion molecule 3, a candidate human immunodeficiency virus type 1 co-receptor on lymphoid and monocytoid cells. J. Gen. Virol. 76, 1345-1352.

Spargo C. A. et al., 1996, Mol. and Cell. Probes, 10:247-256.

Srivastava, M., Fleming, P. J., Pollard, H. B., and Burns, A. L. (1989). Cloning and sequencing of the human nucleolin cDNA. FEBS Letters 250, 99-105.

Stemmer, C., Richalet-Sécordel, P., Van Bruggen, M., Kramers, K., Berden, J., and Muller, S. (1996). Dual reactivity of several monoclonal anti-nucleosome autoantibodies for double-stranded DNA and a short segment of histone H3. J. Biol. Chem. 271, 21257-21261.

Tacson et al., 1996, Nature Medicine, 2(8):888-892.

Tam J. P., 1988, Proc. Nati. Acad. Sci., 85:5409-5413.

Taniguchi, T, and Minami, Y. (1993). The IL-2/IL-2 receptor system: a current overview. Cell 73, 5-8.

Torrence F. et al., 1993, Proc. Natl. Acad. Sci., 90:1300-1304.

Trkola, A., Dragic, T., Arthos, J., Binley, J. M., Olson, W. C., Allaway, G. P., Cheng-Mayer, C., Robinson, J., Maddon, P. J., and Moore, J. P. (1996). CD4dependent antibody-sensitive interactions between HIV-1 and its co-receptor CCR-5. Nature 384, 184-187.

Ugen, K. E., Refaeli, Y., Ziegner, U., Adadjanyan, M., Satre, M. A. R., Srikantan, V., Wang, B., Sato, A., Williams, W. V., and Weiner, D. B. (1993). Generation of monoclonal antibodies against the amino terminus of gp120 that elicit antibody-dependent cellular cytotoxicity. In Vaccines 93, C. S. Harbor, ed., pp. 215-221.

Urdea M. S., 1988, Nucleic Acids Research, 11: 4937-4957.

Vaesen, M., Barnikol-Watanabe, S., Götz, H., Adil Awni, L., Cole, T., Zimmermann, B., Kratzin, H. D., and Hilschmann, N. (1994). Purification and characterization of two putative HLA class II associated proteins: PHAPI and PHAPII. Biol. Chem. Hoppe-Seyler37S, 113-126.

Valadon P., et al., 1996, J. Mol. Biol., 261:11-22.

Valenzuela, A., Blanco, J., Callebaut, C., Jacotot, E., Lluis, C., Hovanessian, A. G., and Franco, R. (1996). Adenosine deaminase binding to human CD26 is inhibited by HIV-1 envelope gp120 and viral particles. J. Immunol. sous presse.

Valenzuela A. et al., 1997, J. Virol., 71: 8289-8298.

Vlasak R. et al., 1983, Eur. J. Biochem., 135:123-126.

von Lindern, M., Fornerod, M., van Baal, S., Jaegle, M., de Wit, T., Buijs, A., and Grosveld, G. (1992). The translocation (6;9), associated with a specific subtype of acute myeloid leukemia, results in the fusion of two genes, dek and can, and the expression of a chimeric, leukemia-specific dek-can mRNA. Mol. Cell. Biol. 12, 1687-1697.

Voss, S. D., Leary, T. P., Sondel, P. M., and Robb, R. J. (1993). Identification of a direct interaction between interleukin 2 and the p64 interleukin 2 receptor gamma chain. Proc Natl Acad Sci USA 90, 2428-32.

Waldmann, T. A. (1993). The IL-2/IL-2 receptor system: a target for rational immune intervention. Immunol. Today 14, 264-270.

Walker G. T. et al., 1992, Nucleic Acids Res., 20:1691-1696.

Westerink M. A. J., 1995, Proc. Natl. Acad. Sci., 92:4021-4025.

Wu D. et al., 1996, J. Pharmacol. and Exp. Therapeutics, 279(1):77-83.

Wu, L., Gerard, N. P., Wyatt, R., Choe, H., Parolin, C., Ruffing, N., Borsetti, A., Cardoso, A. A., Desjardin, E., Newman, W., Gerard, C., and Sodroski, J. (1996). CD4-induced interaction of primary HIV-1 gp120 glycoproteins with the chemokine receptor CCR-5. Nature 384, 179-183.

Yahi et al., 1995, Proc. Natl. Acad. Sci. USA, 92:4867-7.

Yu, Y., Murakami, T., Kawase, S., Uchiyama, T., and Hattori, T. (1995). Characterization of V3 loop-binding protein(s) of Molt-4 and U937 cells. AIDS Res. Hum. Retroviruses 11, 563-570.

Zhenlin et al., Gene, 1989, 78:243-254.

What is claimed is:

1. A novel HIV receptor, named the V3 loop HIV receptor, comprising at least one protein choosen among P95/nucleo/in, P40/PHAPIII and P30/PHAPI proteins.

2. A peptidic or non peptidic inhibitor molecule that is able to modify the interaction between, on one hand the V3 loop receptor according to claim 1 present at the cell surface of a patient infected with a human HIV retrovirus, specifically HIV-1 or HIV-2, and on the other hand the gp120 envelope glycoprotein of said HIV retrovirus.

3. The inhibitor molecule according to claim 2 which comprises a peptide fragment of P95/nucleolin, P40/PHAPII or P30/PHAPI or its pseudopeptide counterpart.

4. The inhibitor molecule of claim 2 which consists in a peptide or pseudopeptide which is homologous containing one or several aminoacid additions, deletions and/or substitutions in the aminoacid sequence of the inhibitor molecules according to claim 3.

5. The inhibitor molecule according to anyone of claims 1 to 4 in which the —CONH— peptide bound is modified and replaced by a (CH₂NH) reduced bound, a (NHCO) retro inverso bound, a (CH₂—O) methylene-oxy bound, a (CH₂—S) thiomethylene bound, a (CH₂CH₂) carba bound, a (CO—CH₂) cetomethylene bound, a (CHOH—CH₂) hydroxyethylene bound), a (N—N) bound, a E-alcene bound or also a —CH═CH— bound.

6. The inhibitor molecule according to anyone of claims 1 to 5, which is derived from the P95/nucleolin aminoacid sequence and choosen among the following sequences:
the sequence beginning at the aminoacid in position 22 and ending at the aminoacid in position 44;
the sequence beginning at the aminoacid in position 143 and ending at the aminoacid in position 171;
the sequence beginning at the aminoacid in position 185 and ending at the aminoacid in position 209;
the sequence beginning at the aminoacid in position 234 and ending at the aminoacid in position 271;

7. The inhibitor molecule according to anyone of claims 1 to 5, which is derived from the P30/PHAPI aminoacid sequence and choosen among the following sequences:
the sequence beginning at the aminoacid in position 168 and ending at the aminoacid in position 182;
the sequence beginning at the aminoacid in position 187 and ending at the aminoacid in position 222;
the sequence beginning at the aminoacid in position 240 and ending at the aminoacid in position 249; it being understood that the proximity of the two first sequences and the two last sequences allow one of ordinary skill in the art to gather the sequences contained in two sets of sequences as follows:
the sequence beginning at the aminoacid in position 168 and ending at the aminoacid in position 222;
the sequence beginning at the aminoacid in position 187 and ending at the aminoacid in position 249;

8. The inhibitor molecule according to anyone of claims 1 to 5, which is the following sequence derived from the P40/PHAPII aminoacid sequence:
the sequence beginning at the aminoacid in position 223 and ending at the aminoacid in position 277

9. The inhibitor molecule according to claim 2 which comprises a polymer of an inhibitor molecule according to anyone of claims 3 to 8, that contains 2 to 20 monomer units of the aminoacid sequence of interest derived from the aminoacid sequence of either P95/nucleolin, P40/PHAPIII and P30/PHAPI, preferably 4 to 15 monomer units and more preferably 5 to 10 monomer units.

10. The inhibitor molecule according to anyone of claims 1 to 9 which is under the form of a MAP matrix structure.

11. The inhibitor molecule according to claim 2 which consists in a monoclonal or polyclonal antibody directed against the P95/nucleolin, P40/PHAPII and P30/PHAPI protein.

12. The inhibitor molecule according to claim 2 which consists in a polyclonal or monoclonal anti-idiotypic antibody that mimmicks the V3 loop peptide of the HIV gp120 glycoprotein.

13. A therapeutic composition comprising a pharmaceutically effective amount of an inhibitor molecule according to anyone of claims 1 to 12, optionally in combination with another anti-HIV molecule such as AZT.

14. A therapeutic composition comprising a pharmaceutically effective amount of a polynucleotide a polynucleotide coding for the P95/nucleolin, P40/PHAPIII and P30/PHAPI or one of the monomeric or oligomeric peptide inhibitor molecules according to anyone of claims 2 to 9.

15. A method of altering the expression of the V3 loop HIV receptor of claim 1 in an individual, which comprises the step of introducing a defect copy of two genes among the genes coding for P95/nucleolin, P40/PHAPIII and P30/PHAPI protein and more preferably a defect copy of the three genes coding for P95/nucleolin, P40/PHAPIII and P30/PHAPI protein in the cells of the individual.

16. A method for specific replacement, in particular by targeting the P95/nucleolin, P40/PHAPIII and P30/PHAPI protein encoding DNA, called insertion DNA, comprising all or part of the DNA structurally encoding for the P95/nucleolin, P40/PHAPIII and P30/PHAPI protein or one of its biologically active derivatives, when it is recombined with a complementing DNA in order to supply a complete recombinant gene in the genome of the host cell of the patient, characterized in that:
the site of insertion is located in a selected gene, called the recipient gene, containing the complementing DNA encoding the P95/nucleolin, P40/PHAPIII and P30/PHAPI protein or one of its biologically active derivatives and in that
the polynucleotide coding for the P95/nucleolin, P40/PHAPIII and P30/PHAPI protein or one of its biologically active derivatives may comprise:
<<flanking sequences>> on either side of the DNA to be inserted, respectively homologous to two genomic sequences which are adjacent to the desired insertion site in the recipient gene.
the insertion DNA being heterologous with respect to the recipient gene, and
the flanking sequences being selected from those which constitute the above-mentioned complementing DNA and which allow, as a result of homologous recombination with corresponding sequences in the recipient gene, the reconstitution of a complete recombinant gene in the genome of the eukaryotic cell.

17. A therapeutic composition comprising an antisense polynucleotide complementary to the nucleic sequence of P95/nucleolin, P40/PHAPIII and P30/PHAPI represented in 49 +L.

18. A method for screening inhibitor molecules according to anyoen of claims 1 to 12 comprising the steps of:
a) Preparing a complex between the P95/nucleolin, P40/PHAPII and P30/PHAPI protein and a ligand that binds to the P95/nucleolin, P40/PHAPII and P30/PHAPI protein by bringing into contact the purified P95/nucleolin, P40/PHAPII and P30/PHAPI protein with a solution containing a molecule to be tested as a ligand binding to the P95/nucleolin, P40/PHAPII and P30/PHAPI protein;
b) visualizing the complex formed between the purified P95/nucleolin, P40/PHAPII and P30/PHAPI protein and the molecule to be tested.

19. A method for screening molecules that modulate the expression of the P95/nucleolin, P40/PHAPII and P30/PHAPI protein, comprising the steps of:
a) cultivating a prokaryotic or an eukaryotic cell that has been transfected with a nucleotide sequence encoding the P95/nucleolin, P40/PHAPII and P30/PHAPI protein, placed under the control of its own promoter;
b) bringing into contact the cultivated cell with a molecule to be tested;
c) quantifying the expression of the P95/nucleolin, P40/PHAPII and P30/PHAPI protein.

20. A method for screening the normal expression of the V3 loop HIV receptor according to the invention comprising the steps of:
a) malting use of monoclonal or polyclonal antibodies directed either to the whole receptor or to the P95/nucleolin, P40/PHAPII and P30/PHAPI protein on isolated patient cells, specifically peripheral mononuclear cells (PMC), said antibodies being optionally radioactively or non radioactively labeled
b) detecting the bound antibodies onto said patients cells.

21. A diagnostic method for detecting mutations in the gene coding for P95/nucleolin, P40/PHAPII or P30/PHAPI comprising the steps of:
a) amplifying the full coding region of P95/nucleolin, P40/PHAPII or P30/PHAPI from a patient using a pair of specific primers;
b) determining the sequence of the amplified DNA;
c) comparing the sequence obtained at step b) with the nucleic sequences of P95/nucleolin, P40/PHAPII or P30/PHAPIreported in 49 +L.

22. A diagnostic nucleic probe comprising at least 20 nucleotides of a mutated sequence of P95/nucleolin, P40/PHAPII or P30/PHAPI, said probe containing at least one specific mutation identified according to the method of claim 21.

23. A method for screening inhibitor according to anyone of claims 2 to 12, comprising the following steps:
a) bringing into contact cells expressing the novel receptor according to the present invention at their surface with an amount of a HIV retrovirus equalling to the TCID₅₀;
b) incubating said cells and retroviruses at 37° C. during a period of time sufficient to allow the entry of the retrovirus within the cells, in the presence of a defined amount of the compound to be assayed,
c) washing the cells in order to remove the retroviruses that has been absorded onto the membranes of the cells;
d) treating the cells in order to eliminate the remaining extracellular retroviruses, for example by a controlled proteolysis with trypsin;
e) preparing cytoplasmic extracts by treating the cells of step d) with an extraction buffer, for example with a buffer containing 20 mM Tris-HCl (pH7.6), 0.15 M NaCl, 5 mM Mg Cl₂, 0.2 mM PMSF, 100 U/ml aprotinin and 0.5% Triton X-100;
f) centrifugating the cells obtained at step c), for example at 1000 g, and harvesting the supernatant medium, in order to seperate the retroviral proteins;
g) detecting and optionally measuring the concentration of the HIV proteins, either directly or indirectly, for example by steric hindering..
